(19) United States
(12) Patent Application Publication
(10) Pub. No.: US 2025/0340529 A1
(43) Pub. Date: Nov. 6, 2025

(54) Heterocyclic Compounds as Immunomodulators of PD-L1 Interactions

(71) Applicant: Ascletis BioScience Co., Ltd, Hangzhou, Zhejiang (CN)

(72) Inventors: Bailing YANG, Hangzhou, Zhejiang (CN); Jinhua CHEN, Hangzhou, Zhejiang (CN); Yang LAI, Hangzhou, Zhejiang (CN); Wei SUN, Hangzhou, Zhejiang (CN); Bin LIANG, Hangzhou, Zhejiang (CN); Liuyu DONG, Hangzhou, Zhejiang (CN); Jiuyong YE, Hangzhou, Zhejiang (CN); Gudmundsson KRISTJAN, Hangzhou, Zhejiang (CN); Jason Jinzi WU, Hangzhou, Zhejiang (CN)

(21) Appl. No.: 18/573,447

(22) PCT Filed: Jul. 2, 2021
(86) PCT No.: PCT/CN2021/104258

§ 371 Date: Dec. 22, 2023

(51) Int. Cl. C07D 401/14; A61K 31/4709; A61K 31/498; A61K 31/538; A61K 31/5383; C07D 265/36; C07D 403/14; C07D 413/14; C07D 471/08; C07D 519/00

(52) U.S. Cl. CPC C07D 401/14; A61K 31/4709; A61K 31/498; A61K 31/538; A61K 31/5383; C07D 265/36; C07D 403/14; C07D 413/14; C07D 471/08; C07D 519/00

(57) ABSTRACT

PD-L1 inhibitors of various compound formulas, both generically and specifically are disclosed. Methods of making such PD-L1 inhibitor compounds are disclosed, both generically and specifically. Methods of using such PD-L1 inhibitor compounds singly or in combination with additional agents and compositions of such PD-L1 inhibitor compounds for the treatment of cancer and other conditions are disclosed.

### FIELD OF THE INVENTION

The present application relates generally to chemical compounds and, in particular, to compounds that modulate the bioactivity of PD-L1 proteins.

### BACKGROUND OF THE INVENTION

Programmed death-ligand 1 (“PD-L1”) is a protein that plays a major role in suppressing the adaptive arm of immune system. Normally the adaptive immune system reacts to antigens that are associated with immune system activation by exogenous or endogenous danger signals. In turn, clonal expansion of antigen-specific CD8+ T cells and/or CD4+ helper cells is propagated. The binding of PD-L1 to the inhibitory checkpoint molecule PD-1 on T cells transmits an inhibitory signal that reduces the proliferation of antigen-specific T-cells in lymph nodes, while simultaneously reducing apoptosis in regulatory T cells (anti-inflammatory, suppressive T cells).

Accordingly, molecules capable of modulating PD-L1 activity may have wide applications in the treatment of various disease conditions.

### SUMMARY OF THE INVENTION

One aspect of the present application relates to a compound that has a generic structure as shown in formula (I):

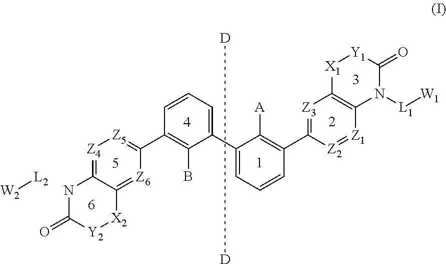

or a pharmaceutically acceptable salt, stereoisomer, mixture of stereoisomers, solvate, or tautomer thereof, wherein,
A and B each is independently selected from halogen, cyano, —N₃, alkyl and substituted alkyl, amine, alkylamine, alkoxy;
Z₁ is —CR¹═ or —N═;
Z₂ is —CR²═;
Z₃ is —CR³═ or —N═;
Z₄ is —CR⁴═ or —N═,
Z₅ is —CR⁵=;
Z₆ is —CR⁶═ or —N═;
R¹ and R⁴ each is independently —H, halogen, cyano, alkyl, cycloalkyl, substituted alkyl, alkenyl, alkenyl, alkynyl, aryl, amine, alkylamine, alkoxy;
R² and R⁵ each is independently, each is independently —H, halogen, cyano, alkyl, cycloalkyl, substituted alkyl, alkenyl, alkynyl, aryl, amine, alkylamine, alkoxy;
R³ and R⁶ each is independently each is independently —H, halogen, cyano, alkyl, cycloalkyl, substituted alkyl, alkenyl, alkynyl, aryl, amine, alkylamine, alkoxy;
Y₁ and Y₂ is independently —C(R⁷)(R⁸)—, —CR⁹═, —NR¹⁰—, —O—, or —S—;
X₁ and X₂ each is independently —C(R¹¹)(R¹²)—, —N═, —NR¹³—, —S— or —O—;
R⁷, R⁸, R⁹, R¹¹, and R¹² each is independently —H, halogen, cyano, alkyl, cycloalkyl, substituted alkyl, alkenyl, alkynyl, aryl, amine, alkylamine, alkoxy;
R¹⁰ and R¹³ each is independently —H, alkyl, cycloalkyl, substituted alkyl, alkenyl, alkenyl, alkynyl, aryl, alkylamine, alkoxy;
L₁ and L₂ each is an alkyl, substituted alkyl or heteroatom chain, containing m atoms between Ring 3 and W₁, and Ring 6 and W₂, wherein m=0, 1, 2, 3, 4, 5 or 6; when m is 0, W₁ or W₂ is directly linked to the corresponding nitrogen in ring 3 or ring 6, respectively;
W₁ and W₂ each is independently hydrogen, a five member heterocyclic ring or substituted five member heterocyclic ring, a six member heterocyclic ring or substituted six member heterocyclic ring, a carboxylalkyl group or substituted carboxylalkyl group, a cyanoalkyl group or substituted cyanoalkyl group, an aminoalkyl group or substituted aminoalkyl group, a hydroxyalkyl group or substituted hydroxyalkyl group, an amino acid, an ester of amino acid, an amide of amino acid, a unnatural amino acid, an ester of unnatural amino acid or an amide of unnatural amino acid.

Another aspect of the present application relates to a method for treating a disease or condition relating to the interaction between PD-L1 and PD-1 in a subject, comprising the step of: administering to the subject, an effective amount of the compound of formula (I), or a pharmaceutically acceptable salt, stereoisomer, mixture of stereoisomers, solvate, or tautomer thereof.

Another aspect of the present application relates to a method for making a compound of formula (I).

### DETAILED DESCRIPTION OF THE INVENTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

At various places in the present specification, certain features of the compounds are disclosed in groups or in ranges. It is specifically intended that such a disclosure include each and every individual subcombination of the members of such groups and ranges.

The compounds described herein can be asymmetric (e.g, having one or more stereocenters). All stereoisomers, such as enantiomers and diastereomers, are intended unless otherwise indicated. Compounds of the present application that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present application. Cis and trans geometric isomers of the compounds of the present application are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as b-camphorsulfonic acid. Other resolving agents suitable for fractional crystallization methods include stereoisomerically pure forms of a-methylbenzylamine (e.g., S and R forms, or diastereomerically pure forms), 2-phenylglycinol, norephedrine, ephedrine, TV-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

In compounds with more than one chiral centers, each of the chiral centers in the compound may be independently (R) or (S), unless otherwise indicated.

Compounds of the application also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, e.g., 1H- and 3/f-imidazole, 1H-, 2H- and 4H-1,2,4-triazole, \H- and 211-isoindole and 1H- and 2//-pyrazole. Tautomeric forms can be in equilibrium or sterically locked into one form by appropriate substitution.

Compounds of the application can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the application can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms.

### I. Definitions

The term, “compound,” as used herein is meant to include all stereoisomers, geometric isomers, tautomers and isotopes of the structures depicted. The term is also meant to refer to compounds of the applications, regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds of the application, or salts thereof, are substantially isolated. By “substantially isolated” is meant that the compound is at least partially or substantially separated from the environment in which it was formed or detected. Partial separation can include, e.g., a composition enriched in the compounds of the application.

Substantial separation can include compositions containing at least about 50%, at least about 60%, at least about 70%, at least about 80%, at least about 90%, at least about 95%, at least about 97%, or at least about 99% by weight of the compounds of the application, or salt thereof.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature,” as used herein, are understood in the art, and refer generally to a temperature, e.g., a reaction temperature, that is about the temperature of the room in which the reaction is carried out, e.g., a temperature from about 20° C. to about 30° C.

The present application also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the non-toxic salts of the parent compound formed, e.g., from non toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred. Lists of suitable salts are found in Remington's Pharmaceutical Sciences, 17th Ed., (Mack Publishing Company, Easton, 1985), p. 1418, Berge et al., J. Pharm. Sci, 1977, 66(1), 1-19 and in Stahl et al., Handbook of Pharmaceutical Salts: Properties, Selection, and Use, (Wiley, 2002). In some embodiments, the compounds described herein include the N-oxide forms.

The terms “individual” or “patient,” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

The term “solvate” refers to the compound formed by the interaction of a solvent and an EPI, a metabolite, or salt thereof. Suitable solvates are pharmaceutically acceptable solvates including hydrates.

The term “substituted” or “optionally substituted” as used in the present invention means that one or more hydrogen atoms of the group to which the term “substitute” or “optionally substituted” refers is replaced with one of the substituents, independently selected from lower alkyl, lower aryl, lower aralkyl, lower cyclic alkyl, lower heterocycloalkyl, hydroxy, lower alkoxy, lower aryloxy, perhaloalkoxy, aralkoxy, lower heteroaryl, lower heteroaryloxy, lower heteroarylalkyl, lower heteroaralkoxy, azido, amino, halo, lower alkylthio, oxo, lower acylalkyl, lower carboxy esters, carboxyl, carboxamido, nitro, lower acyloxy, lower aminoalkyl, lower alkylaminoaryl, lower alkylaryl, lower alkylaminoalkyl, lower alkoxyaryl, lower arylamino, lower aralkylamino, sulfonyl, lower carboxamidoalkylaryl, lower carboxamidoaryl, lower hydroxyalkyl, lower haloalkyl, lower alkylaminoalkylcarboxy-, lower aminocarboxamidoalkyl, cyano, lower alkoxyalkyl, lower perhaloalkyl, and lower arylalkyloxyalkyl, provided that the normal valency of the atom on which the substitution is considered is not exceeded and that the substitution results in a stable chemical compound, that is to say a compound that is sufficiently robust to be isolated from a reaction mixture.

The term “alkyl” refers to a straight or branched or cyclic chain hydrocarbon radical with only single carbon-carbon bonds. Representative examples include methyl, ethyl, propyl, isopropyl, cyclopropyl, butyl, isobutyl, tert-butyl, cyclobutyl, pentyl, cyclopentyl, hexyl, and cyclohexyl, all of which may be optionally substituted.

The term “aryl” refers to aromatic groups which have 5-14 ring atoms and at least one ring having a conjugated pi electron system and includes carbocyclic aryl, heterocyclic aryl and biaryl groups, all of which may be optionally substituted

Carbocyclic aryl groups are groups which have 6-14 ring atoms wherein the ring atoms on the aromatic ring are carbon atoms. Carbocyclic aryl groups include monocyclic carbocyclic aryl groups and polycyclic or fused compounds such as optionally substituted naphthyl groups.

Heterocyclic aryl or heteroaryl groups are groups which have 5-14 ring atoms wherein 1 to 4 heteroatoms are ring atoms in the aromatic ring and the remainder of the ring atoms being carbon atoms. Suitable heteroatoms include oxygen, sulfur, nitrogen, and selenium. Suitable heteroaryl groups include furanyl, thienyl, pyridyl, pyrrolyl, N-lower alkyl pyrrolyl, pyridyl-N-oxide, pyrimidyl, pyrazinyl, imidazolyl, and the like, all optionally substituted.

The term “biaryl” represents aryl groups which have 5-14 atoms containing more than one aromatic ring including both fused ring systems and aryl groups substituted with other aryl groups. Such groups may be optionally substituted. Suitable biaryl groups include naphthyl and biphenyl.

The term “substituted aryl” and “substituted heteroaryl” refers to aryl and heteroaryl groups substituted with 1-3 substituents. These substituents are selected from the group consisting of lower alkyl, lower alkoxy, lower perhaloalkyl, halo, hydroxy, and amino.

The term “aralkyl” refers to an alkylene group substituted with an aryl group. Suitable aralkyl groups include benzyl, picolyl, and the like, and may be optionally substituted.

The term “heteroarylalkyl” refers to an alkylene group substituted with a heteroaryl group.

The term “alkylaryl” refers to an aryl group substituted with an alkyl group. “Lower alkylaryl” refers to such groups where alkyl is lower alkyl.

The term “lower” referred to herein in connection with organic radicals or compounds respectively refers to 6 carbon atoms or less. Such groups may be straight chain, branched, or cyclic.

The term “higher” referred to herein in connection with organic radicals or compounds respectively refers to 7 or more carbon atoms. Such groups may be straight chain, branched, or cyclic.

The term “cyclic alkyl” or “cycloalkyl” refers to alkyl groups that are cyclic of 3 to 10 carbon atoms, and in one aspect are 3 to 6 carbon atoms Suitable cyclic groups include norbornyl and cyclopropyl. Such groups may be substituted.

The term “heterocyclic,” “heterocyclic alkyl” or “heterocycloalkyl” refer to cyclic groups of 3 to 10 atoms, and in one aspect are 3 to 6 atoms, containing at least one heteroatom, in a further aspect are 1 to 3 heteroatoms. Suitable heteroatoms include oxygen, sulfur, and nitrogen. Heterocyclic groups may be attached through a nitrogen or through a carbon atom in the ring. The heterocyclic alkyl groups include unsaturated cyclic, fused cyclic and spirocyclic groups. Suitable heterocyclic groups include pyrrolidinyl, morpholino, morpholinoethyl, and pyridyl.

The terms “arylamino” (a), and “aralkylamino” (b), respectively, refer to the group —NRR′ wherein respectively, (a) R is aryl and R′ is hydrogen, alkyl, aralkyl, heterocycloalkyl, or aryl, and (b) R′ is aralkyl and R′ is hydrogen, aralkyl, aryl, alkyl or heterocycloalkyl.

The term “acyl” refers to —C(O)—R where R is alkyl, heterocycloalkyl, or aryl.

The term “carboxy esters” refers to —C(O)—OR where R is alkyl, aryl, aralkyl, cyclic alkyl, or heterocycloalkyl, all optionally substituted.

The term “carboxyl” refers to —C(O)—OH.

The term “oxo” refers to ═O in an alkyl or heterocycloalkyl group.

The term “amino” refers to —NRR′ where R and R′ are independently selected from hydrogen, alkyl, aryl, aralkyl and heterocycloalkyl, all except H are optionally substituted; and R and R′ can form a cyclic ring system.

The term “carboxylamido” refers to —C(O)NR₂ where each R is independently hydrogen or alkyl.

The term “sulphonylamido” or “-sulfonylamido” refers to —S(═O)₂R₂ where each R is independently hydrogen or alkyl.

The term “halogen” or “halo” refers to —F, —Cl, —Br and —I.

The term “alkylaminoalkylcarboxy” refers to the group alkyl-NR-alk-C(O)—O— where “alk” is an alkylene group, and R is a H or lower alkyl.

The term “sulphonyl” or “sulfonyl” refers to —SO₂R, where R is H, alkyl, aryl, aralkyl, or heterocycloalkyl.

The term “sulphonate” or “sulfonate” refers to —SO₂—OR, where R is —H, alkyl, aryl, aralkyl, or heterocycloalkyl.

The term “alkenyl” refers to unsaturated groups which have 2 to 12 atoms and contain at least one carbon-carbon double bond and includes straight-chain, branched-chain and cyclic groups. Alkenyl groups may be optionally substituted. Suitable alkenyl groups include allyl. “1-Alkenyl” refers to alkenyl groups where the double bond is between the first and second carbon atom. If the 1-alkenyl group is attached to another group, e.g., it is a W substituent attached to the cyclic phosphonate, it is attached at the first carbon.

The term “alkynyl” refers to unsaturated groups which have 2 to 12 atoms and contain at least one carbon-carbon triple bond and includes straight-chain, branched-chain and cyclic groups. Alkynyl groups may be optionally substituted. Suitable alkynyl groups include ethynyl. “1-alkynyl” refers to alkynyl groups where the triple bond is between the first and second carbon atom. If the 1-alkynyl group is attached to another group, e.g., it is a W substituent attached to the cyclic phosphonate, it is attached at the first carbon.

The term “alkylene” refers to a divalent straight chain, branched chain or cyclic saturated aliphatic group. In one aspect the alkylene group contains up to and including 10 atoms. In another aspect the alkylene group contains up to and including 6 atoms. In a further aspect the alkylene group contains up to and including 4 atoms. The alkylene group can be either straight, branched or cyclic.

The term “acyloxy” refers to the ester group —O—C(O)R, where R is H, alkyl, alkenyl, alkynyl, aryl, aralkyl, or heterocycloalkyl.

The term “aminoalkyl” refers to the group NR₂-alk- wherein“alk” is an alkylene group and R is selected from —H, alkyl, aryl, aralkyl, and heterocycloalkyl.

The term “alkylaminoalkyl” refers to the group alkyl-NR-alk- wherein each“alk” is an independently selected alkylene, and R is H or lower alkyl. “Lower alkylaminoalkyl” refers to groups where the alkyl and the alkylene group is lower alkyl and alkylene, respectively.

The term “arylaminoalkyl” refers to the group aryl-NR-alk- wherein “alk” is an alkylene group and R is —H, alkyl, aryl, aralkyl, or heterocycloalkyl. In“lower arylaminoalkyl,” the alkylene group is lower alkylene.

The term “alkylaminoaryl-” refers to the group alkyl-NR-aryl- wherein “aryl” is a divalent group and R is —H, alkyl, aralkyl, or heterocycloalkyl. In “lower alkylaminoaryl,” the alkyl group is lower alkyl.

The term “alkoxyaryl refers to an aryl group substituted with an alkyloxy group. In “lower alkyloxyaryl,” the alkyl group is lower alkyl.

The term “aryloxyalkyl” refers to an alkyl group substituted with an aryloxy group.

The term “aralkyloxyalkyl” refers to the group aryl-alk-O-alk- wherein “alk” is an alkylene group. “Lower aralkyloxyalkyl refers to such groups where the alkylene groups are lower alkylene.

The term “alkoxy-” or “alkyloxy-” refers to the group alkyl-O—.

The term “alkoxyalkyl” or “alkyloxyalkyl” refer to the group alkyl-O-alk- wherein “alk” is an alkylene group. In “lower alkoxyalkyl,” each alkyl and alkylene is lower alkyl and alkylene, respectively.

The term “alkylthio-” refers to the group alkyl-S—.

The term “alkylthioalkyl” refers to the group alkyl-5-alk- wherein “alk” is an alkylene group. In “lower alkylthioalkyl,” each alkyl and alkylene is lower alkyl and alkylene, respectively.

The term “alkoxycarbonyloxy-” refers to alkyl-O—C(O)—O—.

The term “aryloxycarbonyloxy-” refers to aryl-O—C(O)—O—.

The term “alkylthiocarbonyloxy” refers to alkyl-S—C(O)—O—.

The term “amido” refers to the NR₂ group next to an acyl or sulfonyl group as in NR₂—C(O)—, RC(O)—NR¹—, NR₂—S(═O)₂— and RS(═O)₂—NR¹—, where R and R¹ include —H, alkyl, aryl, aralkyl, and heterocycloalkyl

The term “carboxamido” refer to NR₂—C(O)— and RC(O)—NR¹—, where R and R¹ include —H, alkyl, aryl, aralkyl, and heterocycloalkyl. The term does not include urea, —NR—C(O)—NR—.

The term “sulphonamido” or “sulfonamido” refer to NR₂—S(═O)₂— and RS(═O)₂—NR¹—, where R and R¹ include —H, alkyl, aryl, aralkyl, and heterocycloalkyl. The term does not include sulfonylurea, —NR—S(═O)₂—NR—.

The term “carboxamidoalkylaryl” or “carboxamidoaryl” refers to an aryl-alk-NR¹—C(O), and ar-NR¹—C(O)-alk-, respectively where “ar” is aryl, “alk” is alkylene, R¹ and R include II, alkyl, aryl, aralkyl, and heterocycloalkyl.

The term “sulfonamidoalkylaryl” or “sulfonamidoaryl” refers to an aryl-alk-NR¹—S(═O)₂—, and ar-NR¹—S(═O)₂—, respectively where “ar” is aryl, “alk” is alkylene, R¹ and R include —H, alkyl, aryl, aralkyl, and heterocycloalkyl.

The term “hydroxyalkyl” refers to an alkyl group substituted with one —OH.

The term “haloalkyl” refers to an alkyl group substituted with halo.

The term “cyano” refers to —CN.

The term “nitro” refers to —NO₂.

The term “acylalkyl” refers to an alkyl-C(O)-alk-, where“alk” is alkylene.

The term “aminocarboxamidoalkyl” refers to the group NR₂—C(O)—N(R)-alk- wherein R is an alkyl group or H and “alk” is an alkylene group. “Lower aminocarboxamidoalkyl” refers to such groups wherein “alk” is lower alkylen

The term “heteroarylalkyl” refers to an alkylene group substituted with a heteroaryl group.

### II. Compounds that Interfere with PD-L1 Activity

One aspect of the present application relates to compounds that interfere with PD-L1 activity. In some embodiments, the compound has a generic structure as shown in formula (I):

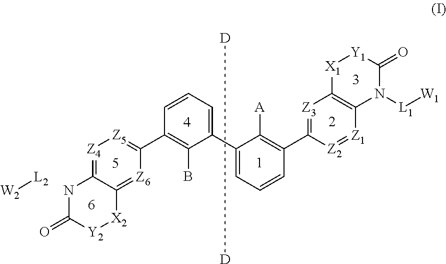

or a pharmaceutically acceptable salt, stereoisomer, mixture of stereoisomers, solvate, or tautomer thereof, wherein,
A and B each is independently selected from halogen, cyano, —N₃, alkyl and substituted alkyl, amine, alkylamine, alkoxy;
Z₁ is —CR¹═ or —N═;
Z₂ is —CR²=;
Z₃ is —CR³═ or —N═;
Z₄ is —CR⁴═ or —N═,
Z₅ is —CR⁵=;
Z₆ is —CR⁶═ or —N═;
R¹ and R⁴ each is independently —H, halogen, cyano, alkyl, cycloalkyl, substituted alkyl, alkenyl, alkenyl, alkynyl, aryl, amine, alkylamine, alkoxy;
R² and R⁵ each is independently, each is independently —H, halogen, cyano, alkyl, cycloalkyl, substituted alkyl, alkenyl, alkynyl, aryl, amine, alkylamine, alkoxy;
R³ and R⁶ each is independently each is independently —H, halogen, cyano, alkyl, cycloalkyl, substituted alkyl, alkenyl, alkynyl, aryl, amine, alkylamine, alkoxy;
Y₁ and Y₂ is independently —C(R⁷)(R⁸)—, —CR⁹═, —NR¹⁰—, —O—, or —S—;
X₁ and X₂ each is independently —C(R¹¹)(R¹²)—, —N═, —NR¹³—, —S— or —O—;
R⁷, R⁸, R⁹, R¹¹, and R¹² each is independently —H, halogen, cyano, alkyl, cycloalkyl, substituted alkyl, alkenyl, alkynyl, aryl, amine, alkylamine, alkoxy;
R¹⁰ and R¹³ each is independently —H, alkyl, cycloalkyl, substituted alkyl, alkenyl, alkenyl, alkynyl, aryl, alkylamine, alkoxy;
L₁ and L₂ each is an alkyl, substituted alkyl or hetereoatom chain, containing m atoms between Ring 3 and W₁, and Ring 6 and W₂, wherein m=0, 1, 2, 3, 4, 5 or 6; when m is 0, W₁ or W₂ is directly linked to the corresponding nitrogen in ring 3 or ring 6, respectively;
W₁ and W₂ each is independently hydrogen, a five member heterocyclic ring or substituted five member heterocyclic ring, a six member heterocyclic ring or substituted six member heterocyclic ring, a carboxylalkyl group or substituted carboxylalkyl group, a cyanoalkyl group or substituted cyanoalkyl group, an aminoalkyl group or substituted aminoalkyl group, a hydroxyalkyl group or substituted hydroxyalkyl group, an amino acid, an ester of amino acid, an amide of amino acid, a unnatural amino acid, an ester of unnatural amino acid or an amide of unnatural amino acid.

The compounds of formula (I) may be symmetrical (i.e., the left portion of the formula (I) is a mirror image of the right portion of formula (I)) or asymmetrical (i.e., the left portion of the formula (I) is different from the right portion of formula (I)) with respect to axis DD.

### Preferred Core Structures

In some embodiments, the compound of formula (I) comprise a core structure selected from the group consisting of formulas (II)—(XXIII):

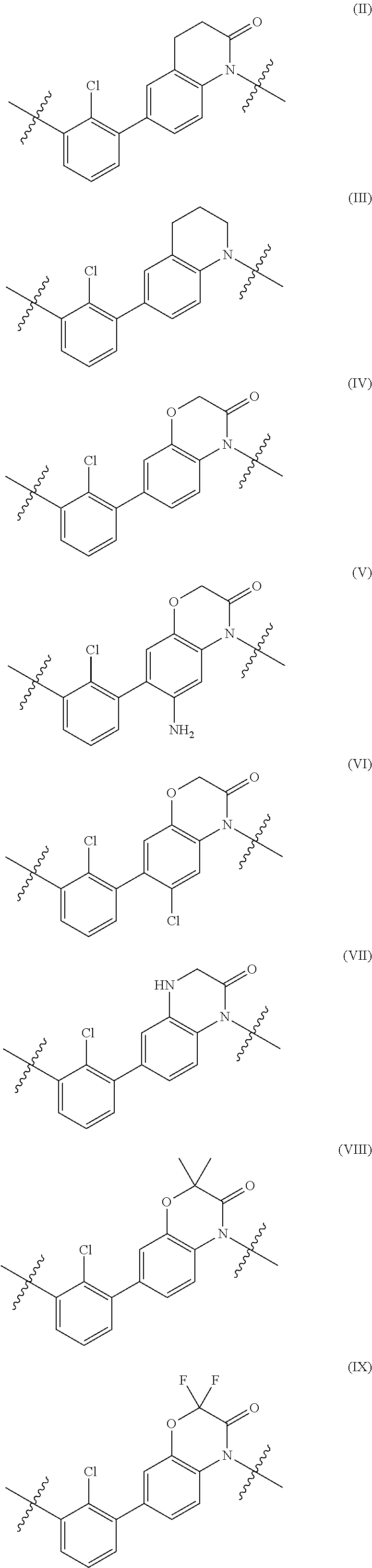

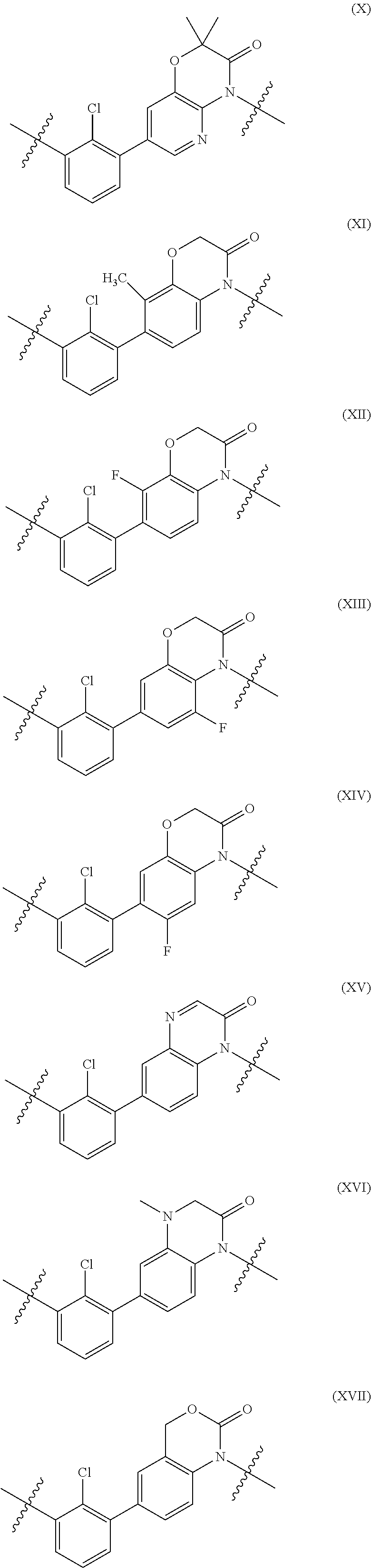

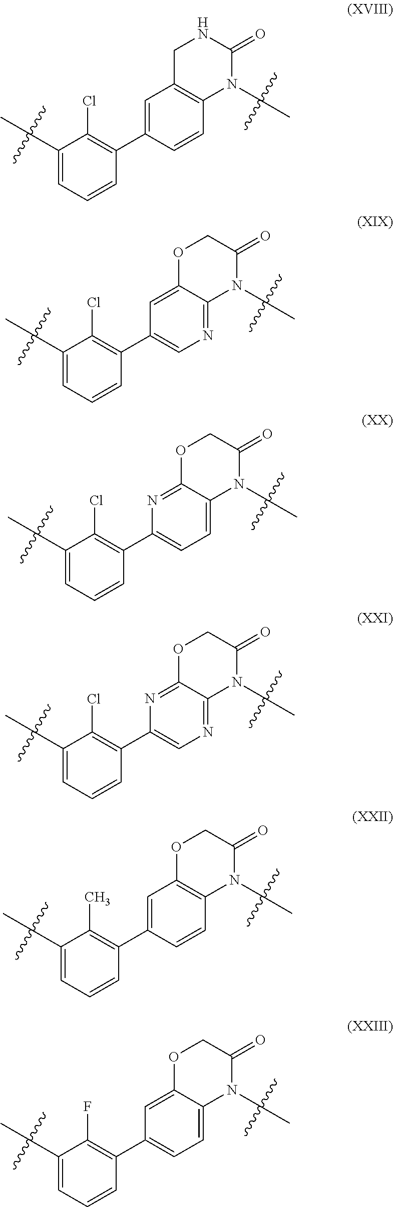 [SMILES: *c1cccc(-c2ccc3c(c2)CNC(=O)N3*)c1Cl ; *c1cccc(-c2cnc3c(c2)OCC(=O)N3*)c1Cl ; *c1cccc(-c2ccc3c(n2)OCC(=O)N3*)c1Cl ; *c1cccc(-c2cnc3c(n2)OCC(=O)N3*)c1Cl ; *c1cccc(-c2ccc3c(c2)OCC(=O)N3*)c1C ; *c1cccc(-c2ccc3c(c2)OCC(=O)N3*)c1F]

In some embodiments, the compound of formula (I) comprises the core structures of:

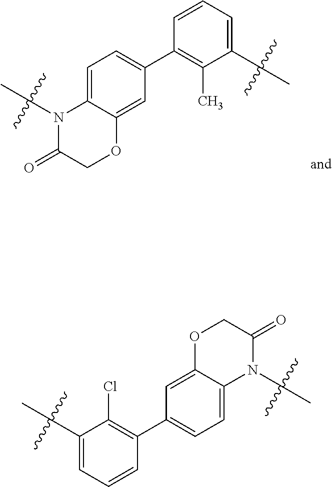 [SMILES: *c1cccc(-c2ccc3c(c2)OCC(=O)N3*)c1C ; *c1cccc(-c2ccc3c(c2)OCC(=O)N3*)c1Cl]

In some embodiments, L₁ and L₂ each is independently selected from —CH₂—, (CH₂)₂— and —(CH₂)₃—.

In some embodiments, L₁ and L₂ each is independently selected from —CH(CH₃)—, —CH₂— and —CH₂—CH(CH₃)—.

### Type I Side Chains

In some embodiments, W₁ and/or W₂ each is independently a type I side chain. As used herein, the term “type I side chain” refers to a structure that contains (1) a five-member heterocyclic ring having at least one nitrogen atom as the ring atom or a substituted five-member heterocyclic ring having at least one nitrogen atom as the ring atom, or (2) a six-member heterocyclic ring having at least one nitrogen atom as a ring atom or a substituted five-member heterocyclic ring having at least one nitrogen atom as a ring atom, wherein linker L₁ or L₂ is linked directly to a ring atom in the five-member or six member heterocyclic ring.

In some embodiments, W₁ and/or W₂ each is independently a heterocyclic ring. In some embodiments, W₁ and/or W₂ each is independently a five member heterocyclic ring having at least one nitrogen atom as a ring atom. In some embodiments, W₁ and/or W₂ each is independently a six member heterocyclic ring having at least one nitrogen atom as a ring atom.

In some embodiments, W₁ and/or W₂ each is independently selected from the group of type I side chains listed below:

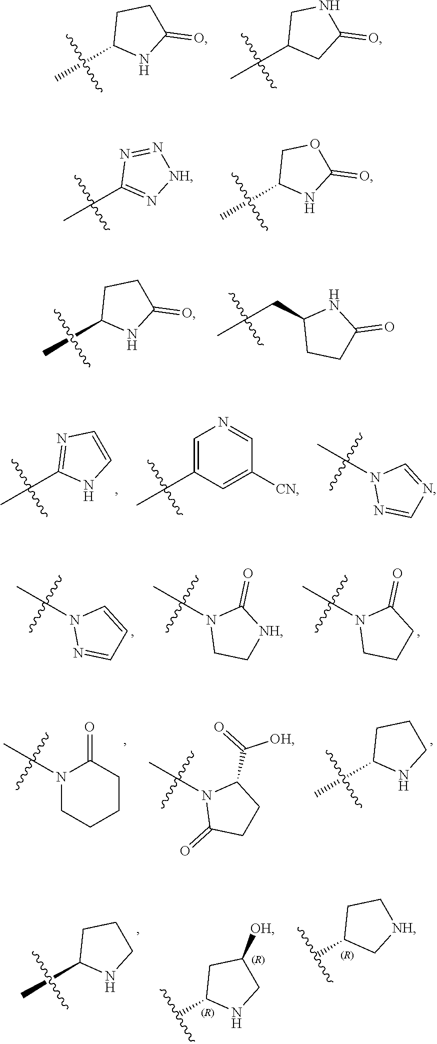

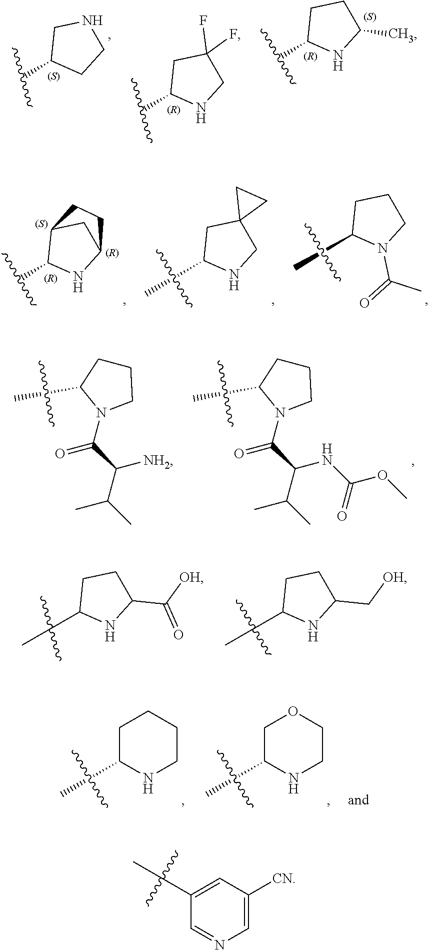

### Type 2 Side Chains

In some embodiments, W₁ and/or W₂ each is independently a type II side chain. As used herein, the term “type II side chain” refers to a W₁ structure having a general formula of:

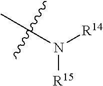

and a W₂ structure having a general formula of:

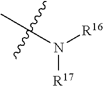

wherein R¹⁴ and R¹⁶ each is independently —H, an alkyl, a substituted alkyl, a hydroxyalkyl or a substituted hydroxyalkyl, a hydroxycarboxyl acid or a salt or an ester thereof, a substituted hydroxycarboxyl acid or a salt or an ester thereof, a carboxyl acid or a salt or a ester or an alkyl ether thereof, a substituted carboxyl acid or a salt or a ester or an alkyl ether thereof, a carboamide, or a lactone; and
wherein R¹⁵ and R¹⁷ each is independently —H, an alkyl, or a substituted alkyl.

In some embodiments, R¹⁴ or R¹⁶ or both have the general formula of -L₃-C(O)-Q₂R¹⁸, wherein L₃ is an alkyl, substituted alkyl, alkylamino or alkly-amino-alkyl, Q₂ is —O— or —CH₂—, and R¹⁸ is —H, alkyl or substituted alkyl.

In some embodiment, R¹⁴ or R¹⁶ or both are independently selected from the group consisting of

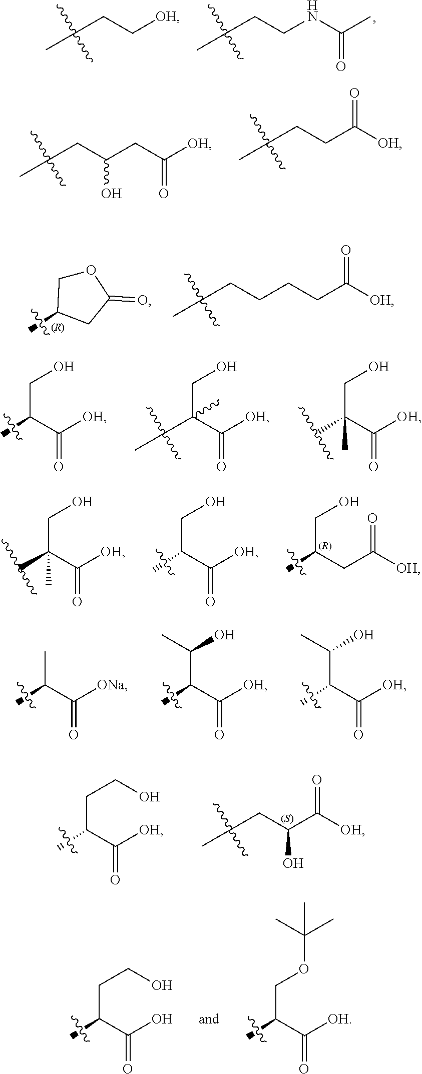

In some embodiments, W₁ or W₂ each is independently an amino acid.

In some embodiments, the side-chain W₁ or W₂ is L-serine

In some embodiments, the side-chain W₁ or W₂ is a L-serine ester.

In some embodiments, the side-chains W₁ and W₂ are both L-serine

In some embodiments, the side-chain W₁ and W₂ are both L-serine esters.

### Other Side Chains

In some embodiments, W₁ or W₂ each is independently —C(O)—ONa, —CN, —CH₂OH or —CH₂NH₂.

### Exemplary Compound

In some embodiments, the compound of formula (I) comprise the core structure of

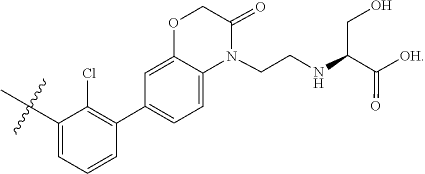 [SMILES: *c1cccc(-c2ccc3c(c2)OCC(=O)N3CCN[C@@H](CO)C(=O)O)c1Cl]

In some embodiments, the compound of formula (I) comprise the core structure of

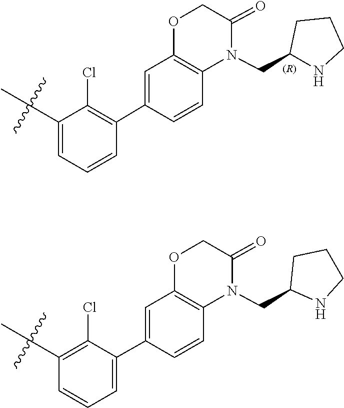

In some embodiments, the compound of formula (I) consists of two identical core structure of

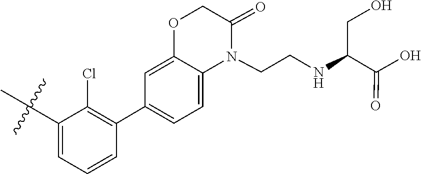 [SMILES: *c1cccc(-c2ccc3c(c2)OCC(=O)N3CCN[C@@H](CO)C(=O)O)c1Cl]

linked together.

In some embodiments, the compound of formula (I) consists of two identical core structure of

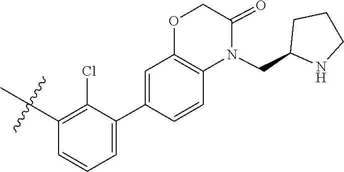 [SMILES: *c1cccc(-c2ccc3c(c2)OCC(=O)N3C[C@H]2CCCN2)c1Cl]

linked together.

In some embodiments, the compound of formula (I) consists of the core structure of

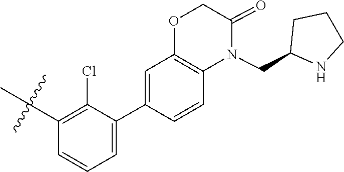 [SMILES: *c1cccc(-c2ccc3c(c2)OCC(=O)N3C[C@H]2CCCN2)c1Cl]

and the core structure of

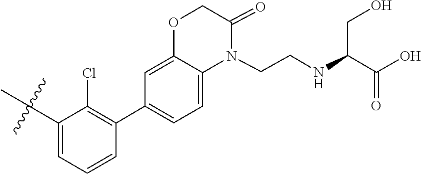 [SMILES: *c1cccc(-c2ccc3c(c2)OCC(=O)N3CCN[C@@H](CO)C(=O)O)c1Cl]

linked together.

In some embodiments, the side-chains W₁ and/or W₂ is independently selected from the group consisting of:

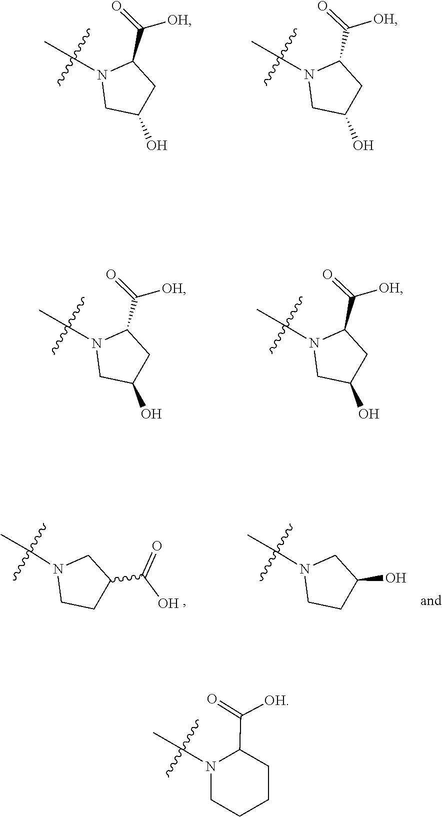

In some embodiments, the compound of formula (I) comprises only one side-chain, wherein:
W₁ is

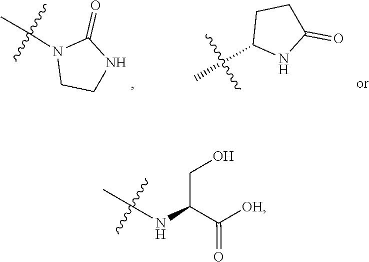

W₂ is H, and L₂ is absent (i.e., m=0). In other words, there is no side-chain at the nitrogen atom in ring 6. In a further embodiment, L₁ is a C₁-C₃ alkyl.

In some embodiments, the compound of formula (I) comprises side-chains that are asymmetrical over axis DD.

In some embodiments, W₁ is

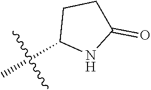

and W₂ is selected from type I and type II side chains. In a further embodiment, L₁ and L₂ each is independently a C₁-C₃ alkyl.

In some embodiments, W₁ is

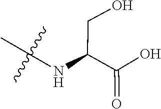 [SMILES: *N[C@@H](CO)C(=O)O]

and W₂ is selected from type I and type II side chains. In a further embodiment, L₁ and L₂ each is independently a C₁-C₃ alkyl.

In some embodiments, W₁ is

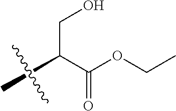

and W₂ is selected from type I and type II side chains. In a further embodiment, L₁ and L₂ each is independently a C₁-C₃ alkyl.

In some embodiments, W₁ is

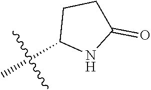

### and W₂ is

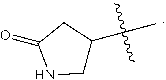 [SMILES: *C1CNC(=O)C1]

In a further embodiment, L₁ and L₂ each is independently a C₁-C₃ alkyl.

In some embodiments, W₁ is

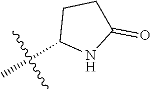

### and W₂ is

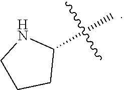

In a further embodiment, L₁ and L₂ each is independently a C₁-C₃ alkyl.

In some embodiments, W₁ is

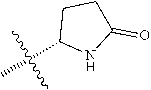

### and W₂ is

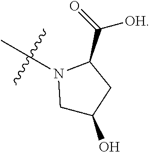 [SMILES: *N1C[C@H](O)C[C@@H]1C(=O)O]

In a further embodiment, L₁ and L₂ each is independently a C₁-C₃ alkyl.

In some embodiments, side chain W₁ is

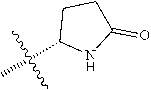

and side chain W₂ is

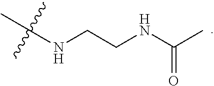 [SMILES: *NCCNC(C)=O]

In a further embodiment, L₁ and L₂ each is independently a C₁-C₃ alkyl.

In some embodiments, side chain W₁ is

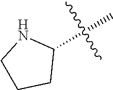

and side chain W₂ is

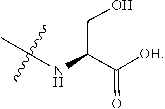 [SMILES: *N[C@@H](CO)C(=O)O]

In a further embodiment, L₁ and L₂ each is independently a C₁-C₃ alkyl.

In some embodiments, side chain W₁ is

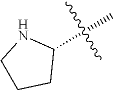

and side chain W₂ is

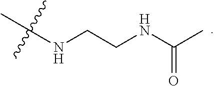 [SMILES: *NCCNC(C)=O]

In a further embodiment, L₁ and L₂ each is independently a C₁-C₃ alkyl.

In some embodiments, side chain W₁ is

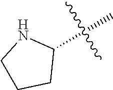

and side chain W₂ is

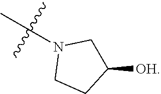 [SMILES: *N1CC[C@H](O)C1]

In a further embodiment, L₁ and L₂ each is independently a C₁-C₃ alkyl.

In some embodiments, side chain W₁ is

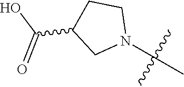 [SMILES: *N1CCC(C(=O)O)C1]

and side chain W₂ is

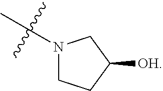 [SMILES: *N1CC[C@H](O)C1]

In a further embodiment, L₁ and L₂ each is independently a C₁-C₃ alkyl.

In some embodiments, side chain W₁ is

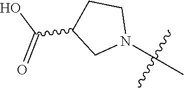 [SMILES: *N1CCC(C(=O)O)C1]

and side chain W₂ is

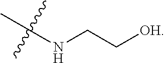 [SMILES: *NCCO]

In a further embodiment, L₁ and L₂ each is independently a C₁-C₃ alkyl.

In some embodiments, side chain W₁ is

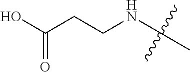 [SMILES: *NCCC(=O)O]

and side chain W₂ is

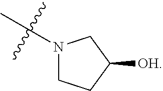 [SMILES: *N1CC[C@H](O)C1]

In a further embodiment, L₁ and L₂ each is independently a C₁-C₃ alkyl.

Certain embodiments of the compounds disclosed herein are listed, without limitation, in Table 1 (or a pharmaceutically acceptable salt, stereoisomer, mixture of stereoisomers, solvate, or tautomer thereof):

TABLE 1

| Compound | | |
|---|---|---|
| No. | Structure | [M + H]⁺ |
| 281 | 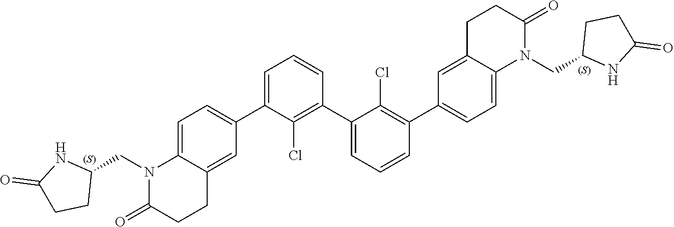 | 707.43 |
| 303 | 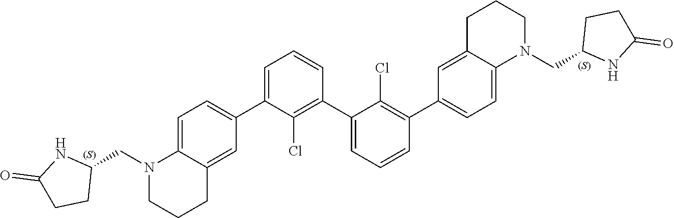 | 679.45 |
| 448 | 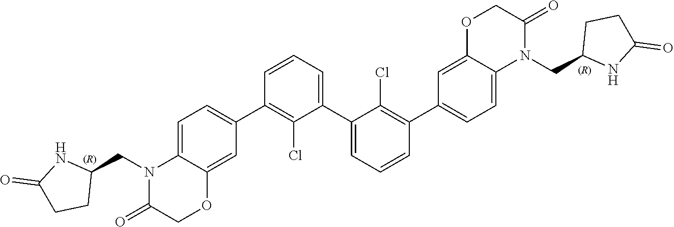 | 711.47 |
| 338 | 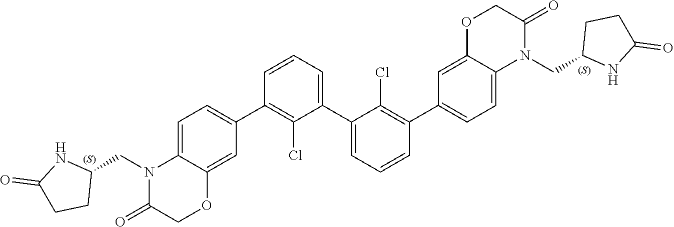 | 711.50 |
| 354 | 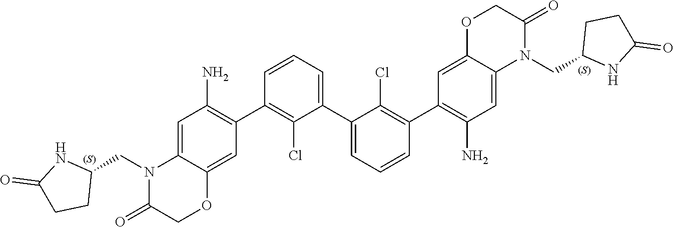 | 741.50 |
| 361 | 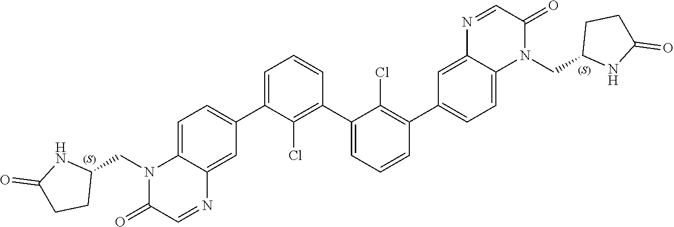 | 705.52 |
| 363 | 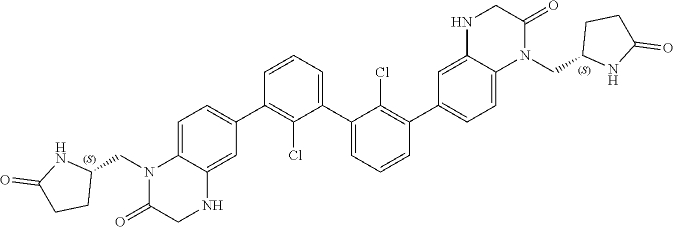 | 709.44 |
| 372 | 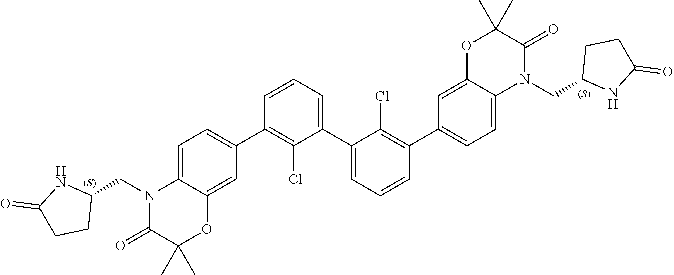 | 767.40 |
| 376 | 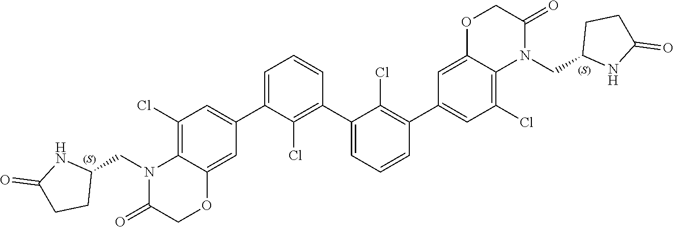 | 779.41 |
| 381 | 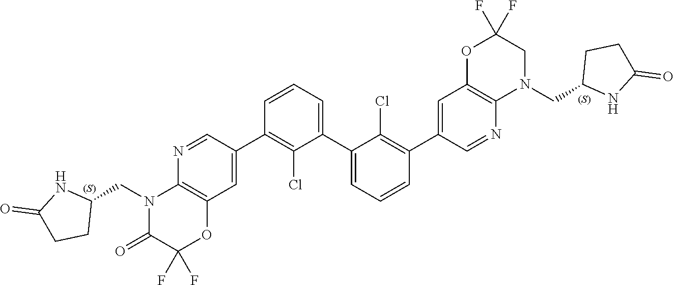 | 785.12 |
| 382 | 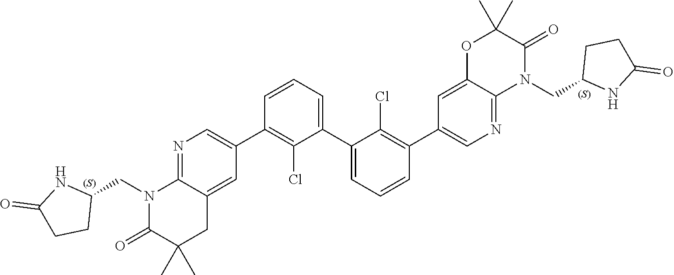 | 769.55 |
| 388 | 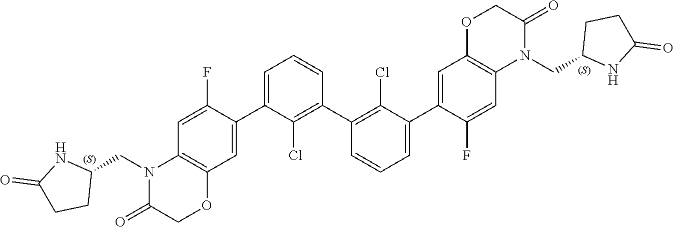 | 747.10 |
| 395 | 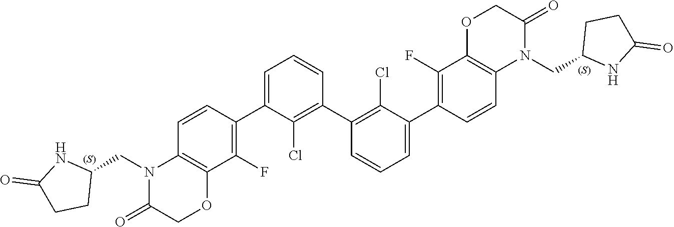 | 747.11 |
| 396 | 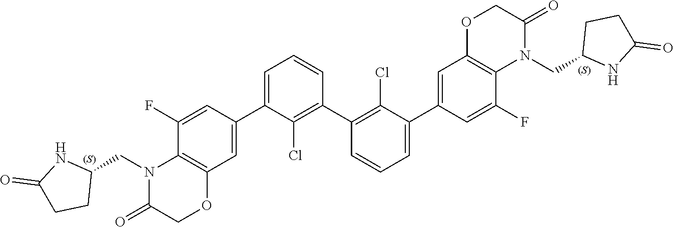 | 747.15 |
| 401 | 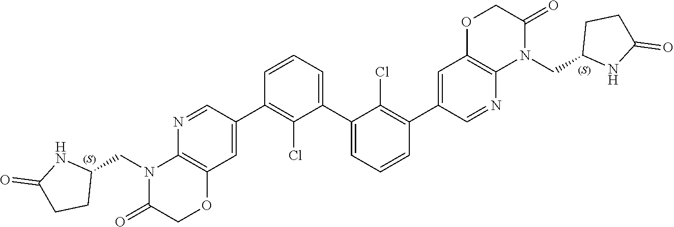 | 713.16 |
| 402 | 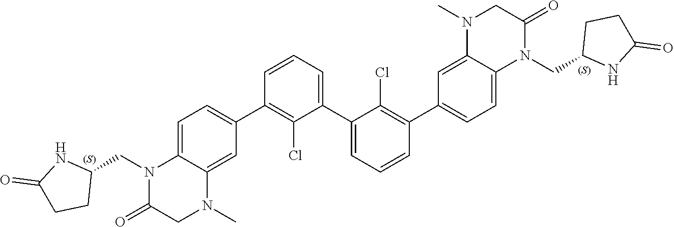 | 737.52 |
| 457 | 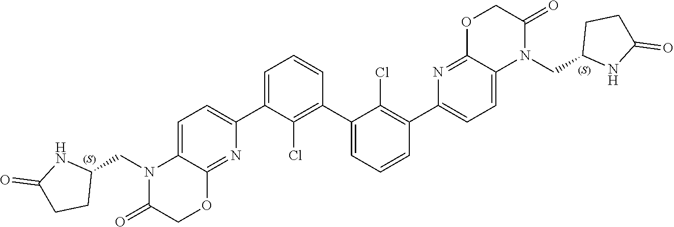 | 713.48 |
| 466 | 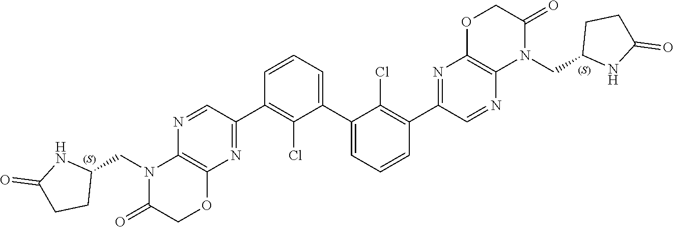 | 715.15 |
| 481 | 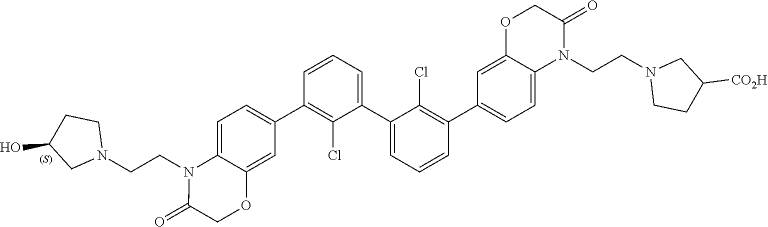 | 771.50 |
| 445 | 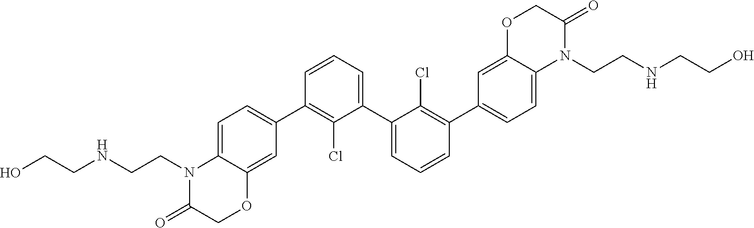 [SMILES: O=C1COc2cc(-c3cccc(-c4cccc(-c5ccc6c(c5)OCC(=O)N6CCNCCO)c4Cl)c3Cl)ccc2N1CCNCCO] | 691.2 |
| 449 | 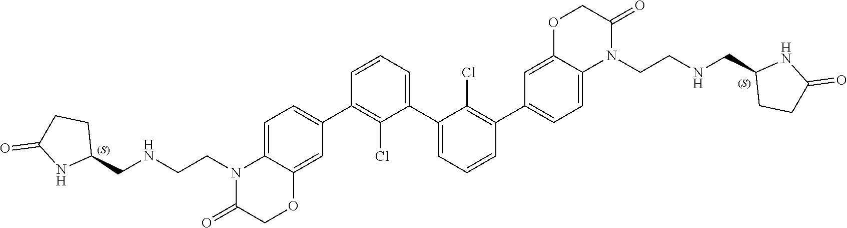 | 797.3 |
| 454 | 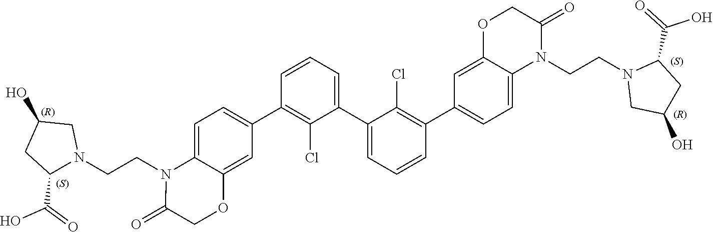 | 831.45 |
| 460 | 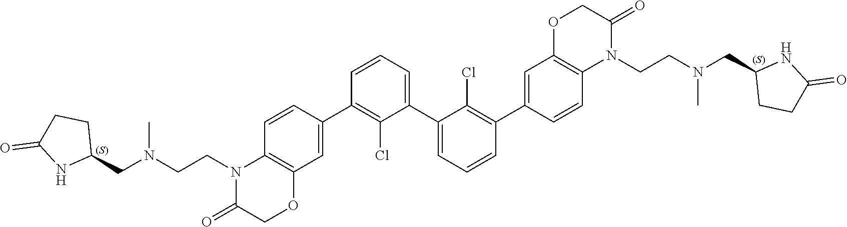 | 825.3 |
| 461 | 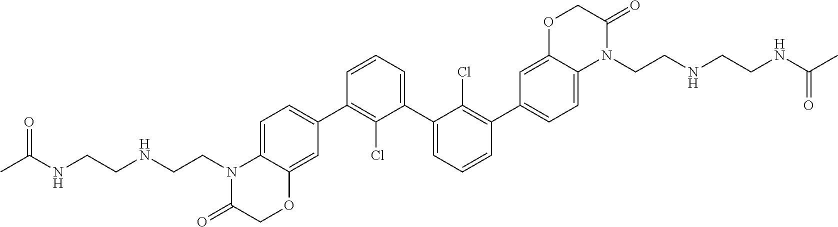 [SMILES: CC(=O)NCCNCCN1C(=O)COc2cc(-c3cccc(-c4cccc(-c5ccc6c(c5)OCC(=O)N6CCNCCNC(C)=O)c4Cl)c3Cl)ccc21] | 773.55 |
| 465 | 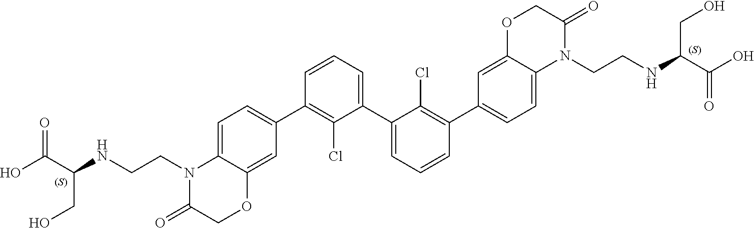 | 779.25 |
| 468 | 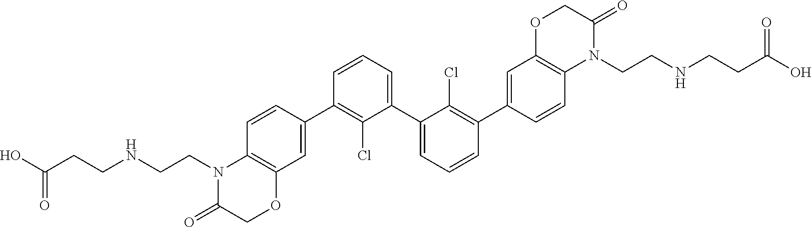 [SMILES: O=C(O)CCNCCN1C(=O)COc2cc(-c3cccc(-c4cccc(-c5ccc6c(c5)OCC(=O)N6CCNCCC(=O)O)c4Cl)c3Cl)ccc21] | 747.2 |
| 469 | 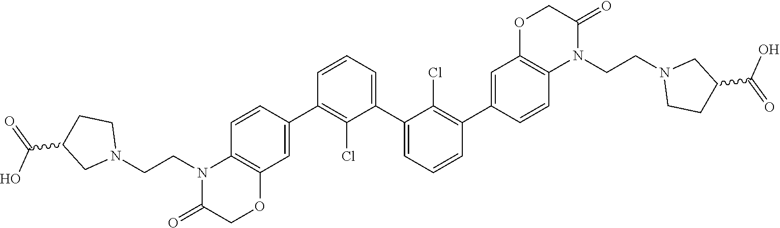 [SMILES: O=C(O)C1CCN(CCN2C(=O)COc3cc(-c4cccc(-c5cccc(-c6ccc7c(c6)OCC(=O)N7CCN6CCC(C(=O)O)C6)c5Cl)c4Cl)ccc32)C1] | 799.2 |
| 470 | 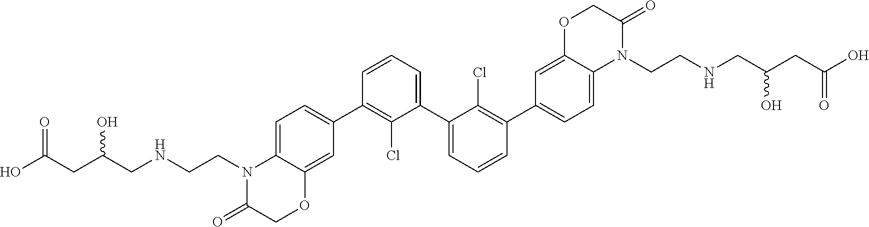 [SMILES: O=C(O)CC(O)CNCCN1C(=O)COc2cc(-c3cccc(-c4cccc(-c5ccc6c(c5)OCC(=O)N6CCNCC(O)CC(=O)O)c4Cl)c3Cl)ccc21] | 807.25 |
| 471 | 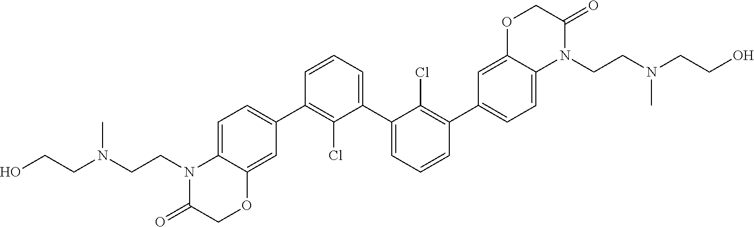 [SMILES: CN(CCO)CCN1C(=O)COc2cc(-c3cccc(-c4cccc(-c5ccc6c(c5)OCC(=O)N6CCN(C)CCO)c4Cl)c3Cl)ccc21] | 719.52 |
| 475 | 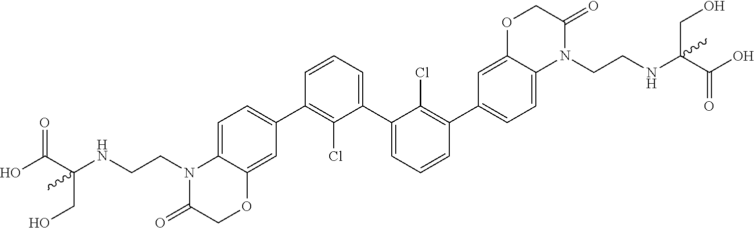 | 807.41 |
| 476 | 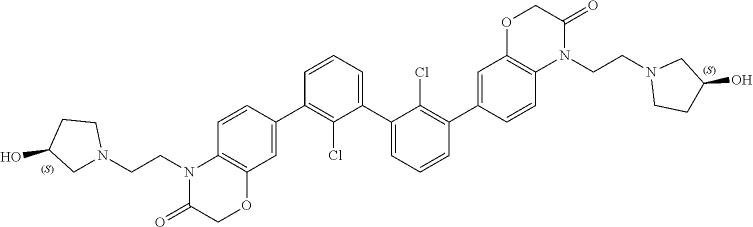 | 743.2 |
| 478 | 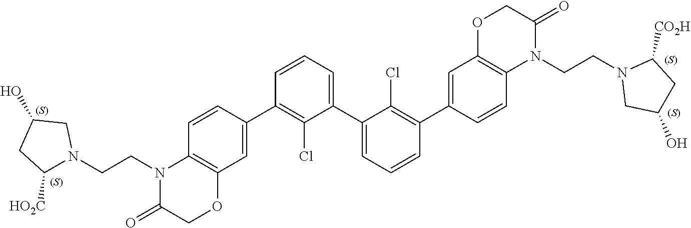 | 831.2 |
| 482 | 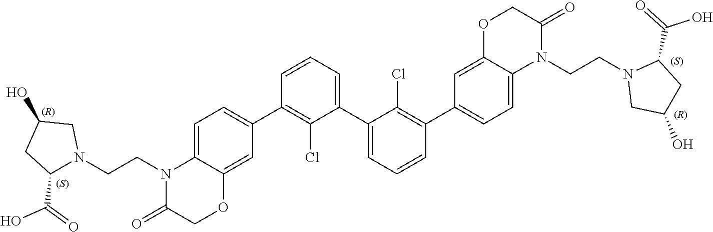 | 831.50 |
| 483 | 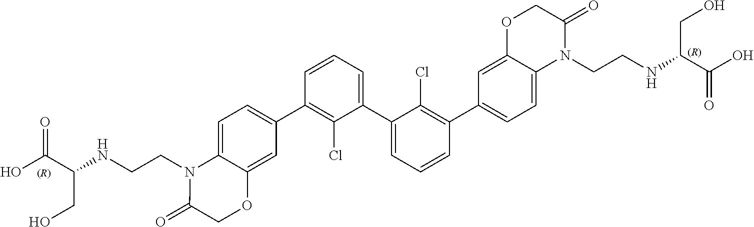 | 779.25 |
| 484 | 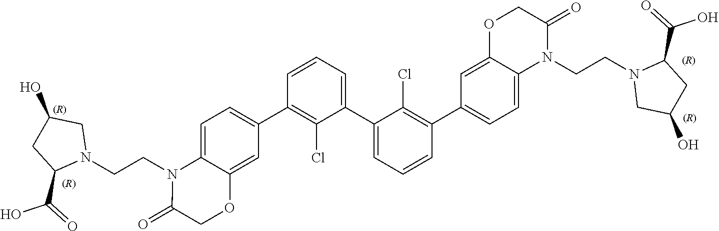 | 831.45 |
| 490 | 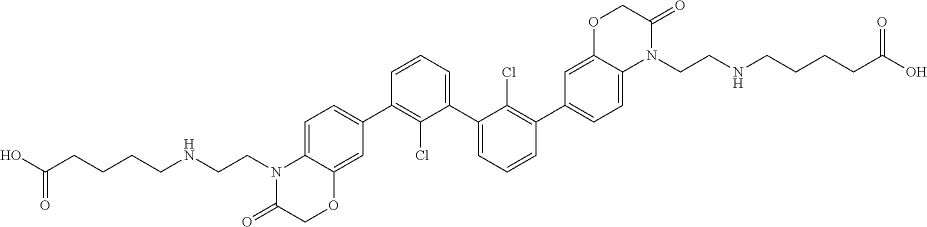 [SMILES: O=C(O)CCCCNCCN1C(=O)COc2cc(-c3cccc(-c4cccc(-c5ccc6c(c5)OCC(=O)N6CCNCCCCC(=O)O)c4Cl)c3Cl)ccc21] | 803.51 |
| 491 | 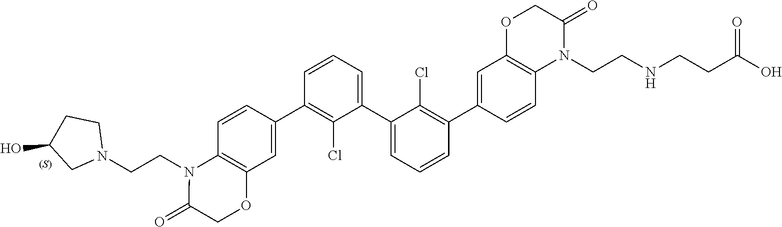 | 745.2 |
| 493 | 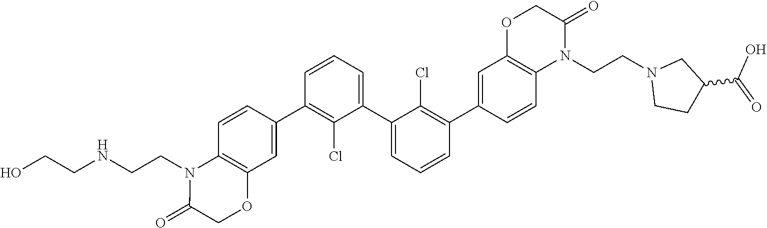 [SMILES: O=C(O)C1CCN(CCN2C(=O)COc3cc(-c4cccc(-c5cccc(-c6ccc7c(c6)OCC(=O)N7CCNCCO)c5Cl)c4Cl)ccc32)C1] | 745.2 |
| 513 | 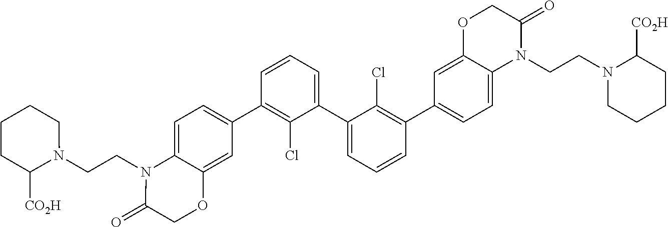 [SMILES: O=C(O)C1CCCCN1CCN1C(=O)COc2cc(-c3cccc(-c4cccc(-c5ccc6c(c5)OCC(=O)N6CCN5CCCCC5C(=O)O)c4Cl)c3Cl)ccc21] | 827.50 |
| 527 | 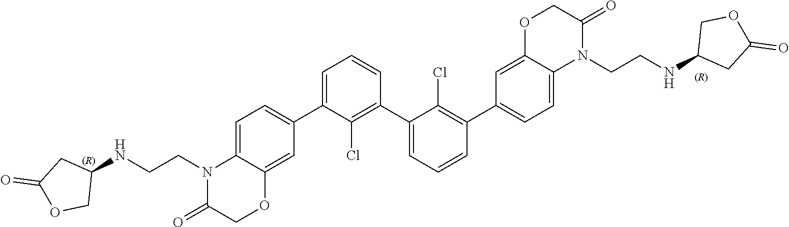 | 771.48 |
| 528 | 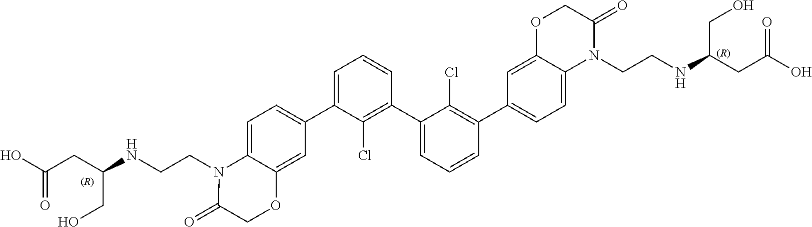 | 807.45 |
| 536 | 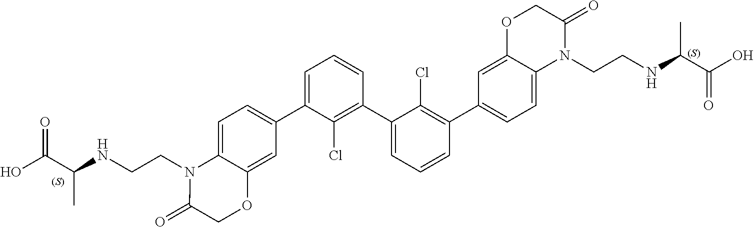 | 747.41 |
| 537 | 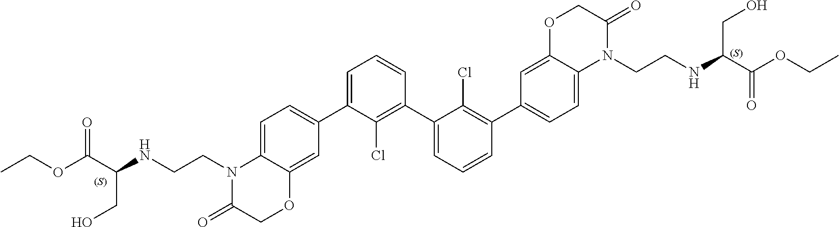 | 835.51 |
| 541 | 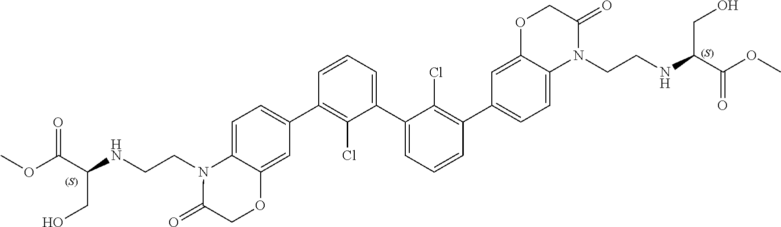 | 807.49 |
| 545 | 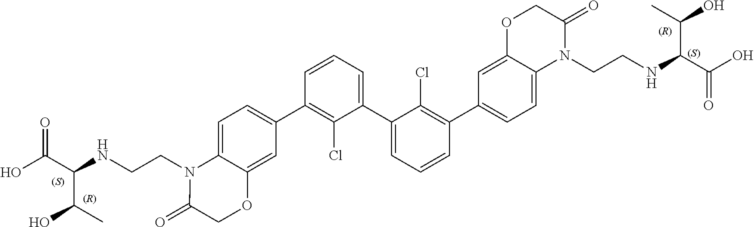 | 807.21 |
| 551 | 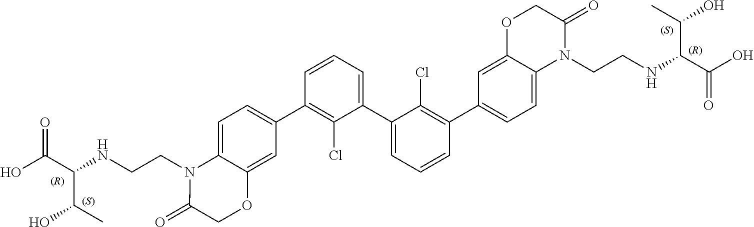 | 807.21 |
| 552 | 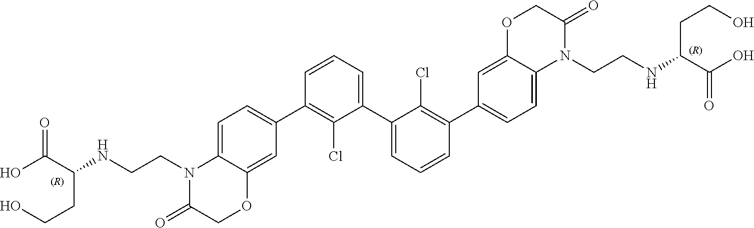 | 807.21 |
| 553 | 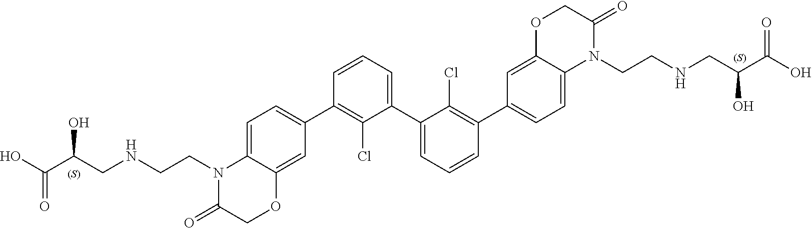 | 779.40 |
| 560 | 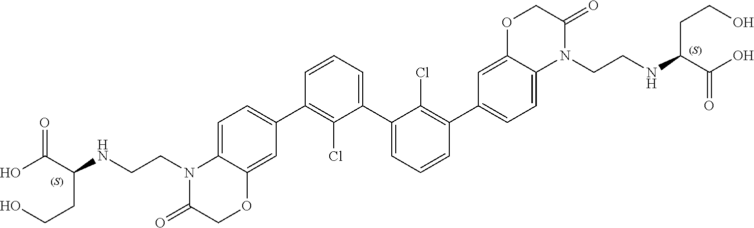 | 807.21 |
| 566 | 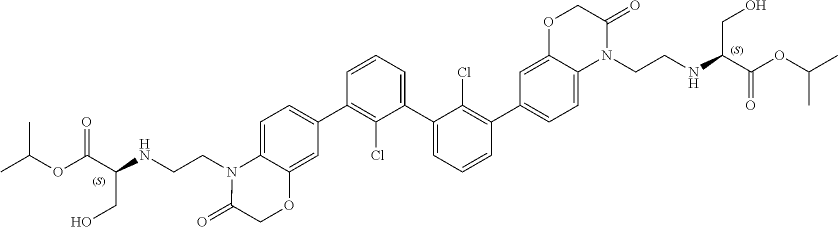 | 863.77 |
| 570 | 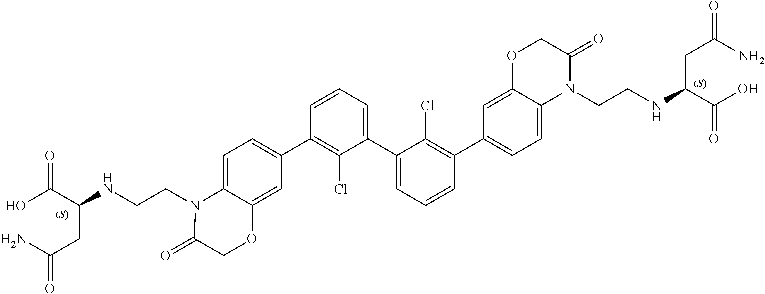 | 833.20 |
| 571 | 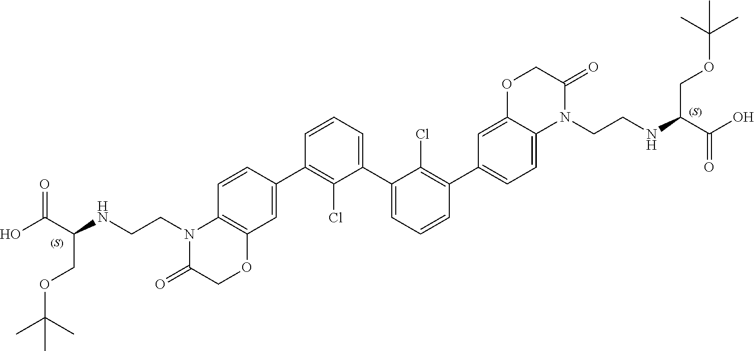 | 891.31 |
| 573 | 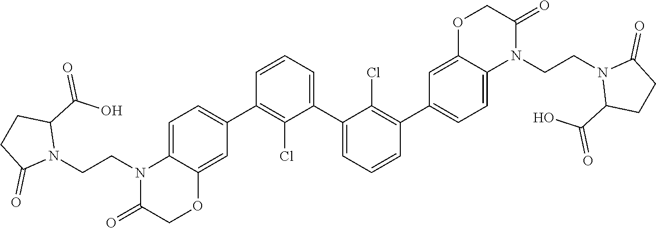 [SMILES: O=C(O)C1CCC(=O)N1CCN1C(=O)COc2cc(-c3cccc(-c4cccc(-c5ccc6c(c5)OCC(=O)N6CCN5C(=O)CCC5C(=O)O)c4Cl)c3Cl)ccc21] | 827.18 |
| 588 | 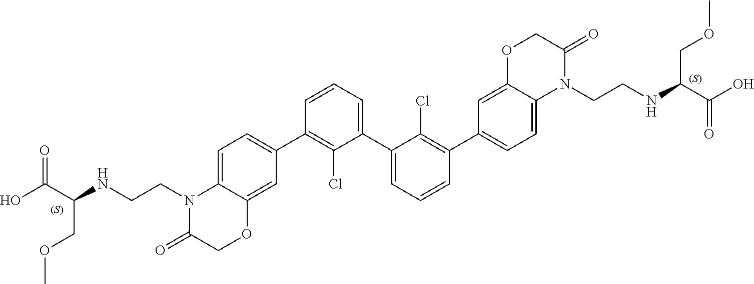 | 807.21 |
| 479 | 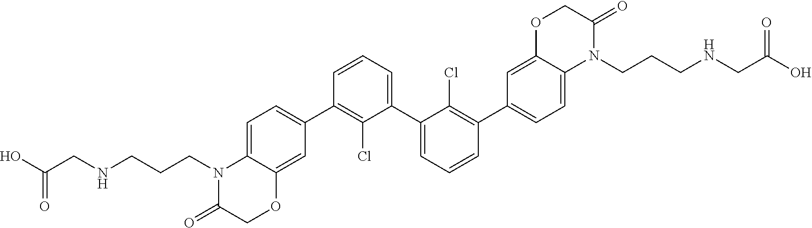 [SMILES: O=C(O)CNCCCN1C(=O)COc2cc(-c3cccc(-c4cccc(-c5ccc6c(c5)OCC(=O)N6CCCNCC(=O)O)c4Cl)c3Cl)ccc21] | 747.46 |
| 480 | 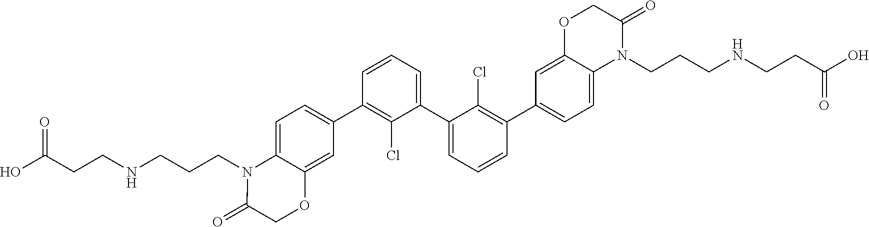 [SMILES: O=C(O)CCNCCCN1C(=O)COc2cc(-c3cccc(-c4cccc(-c5ccc6c(c5)OCC(=O)N6CCCNCCC(=O)O)c4Cl)c3Cl)ccc21] | 775.50 |
| 563 | 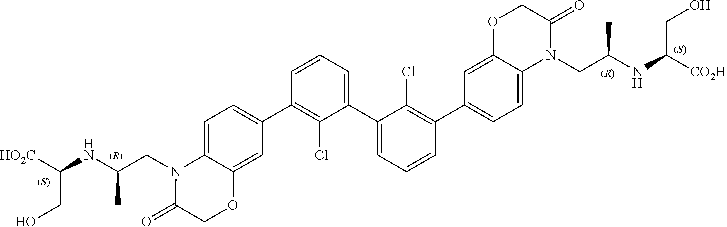 | 807.43 |
| 550 | 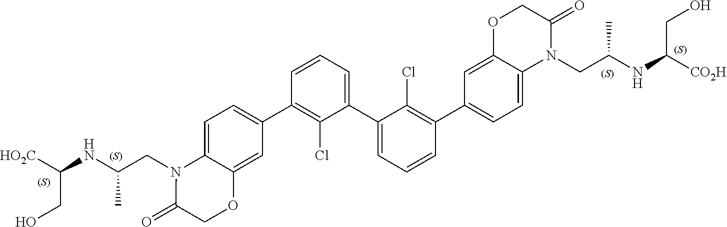 | 807.43 |
| 554 | 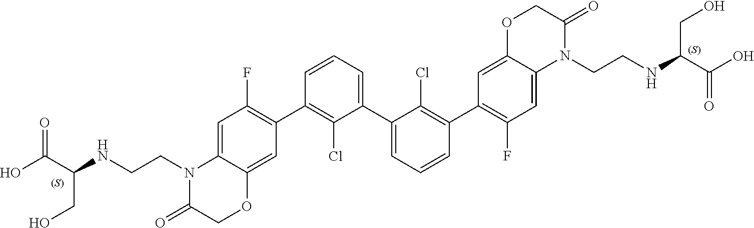 | 815.33 |
| 589 | 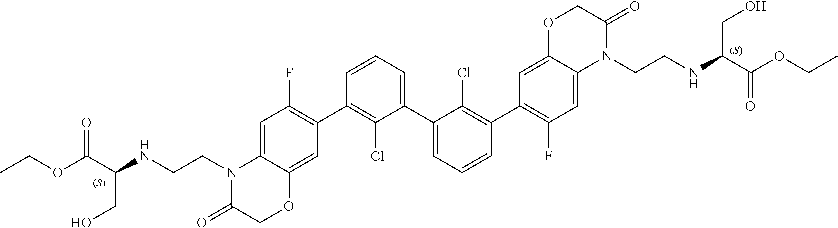 | 871.50 |
| 561 | 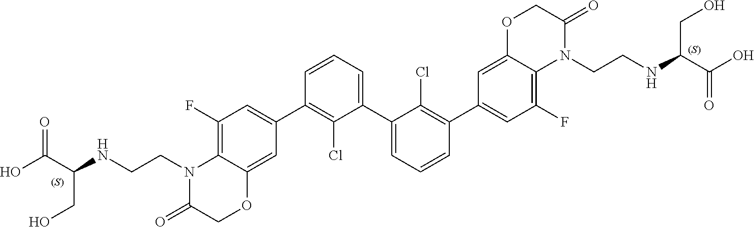 | 815.16 |
| 557 | 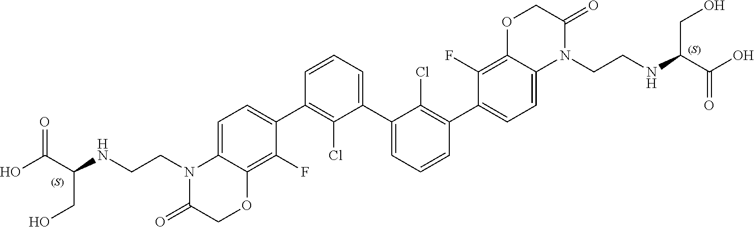 | 815.16 |
| 586 | 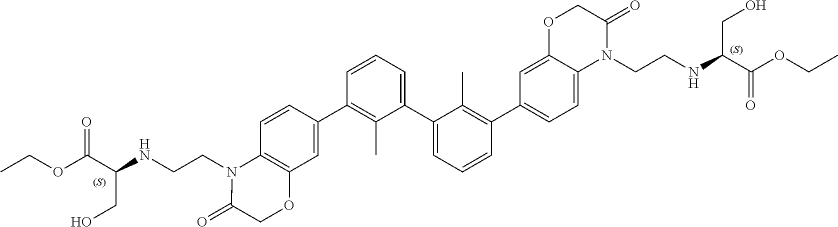 | 795.35 |
| 585 | 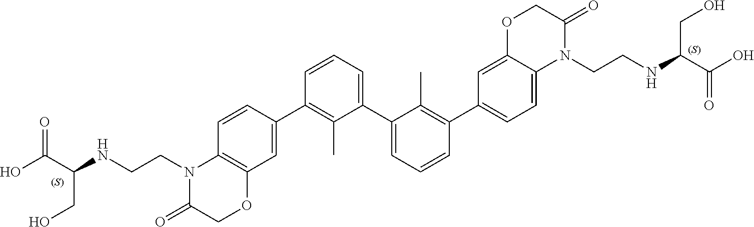 | 739.50 |
| 514 | 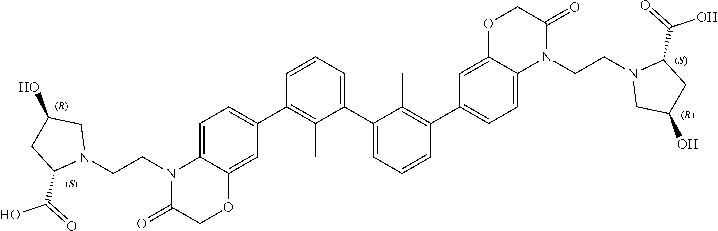 | 791.58 |
| 373 | 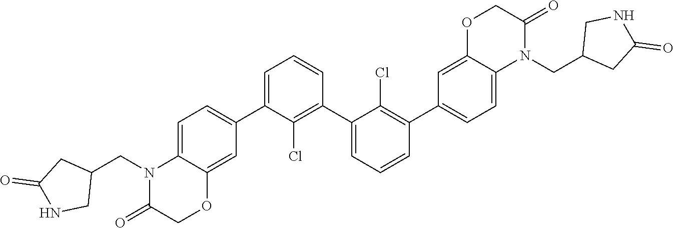 [SMILES: O=C1CC(CN2C(=O)COc3cc(-c4cccc(-c5cccc(-c6ccc7c(c6)OCC(=O)N7CC6CNC(=O)C6)c5Cl)c4Cl)ccc32)CN1] | 711.47 |
| 374 | 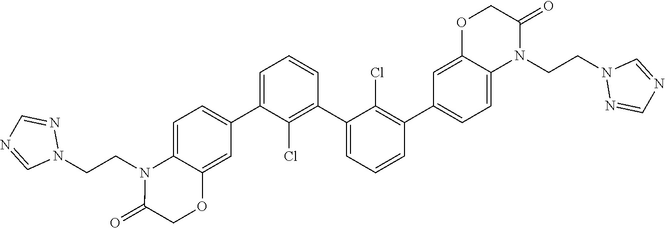 [SMILES: O=C1COc2cc(-c3cccc(-c4cccc(-c5ccc6c(c5)OCC(=O)N6CCn5cncn5)c4Cl)c3Cl)ccc2N1CCn1cncn1] | 707.43 |
| 375 | 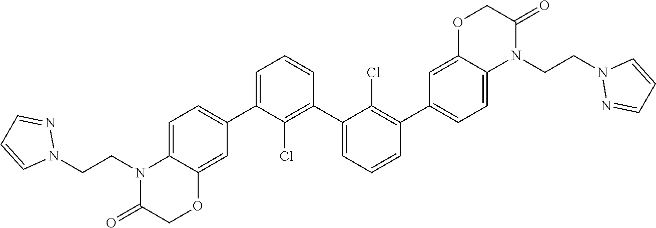 [SMILES: O=C1COc2cc(-c3cccc(-c4cccc(-c5ccc6c(c5)OCC(=O)N6CCn5cccn5)c4Cl)c3Cl)ccc2N1CCn1cccn1] | 705.42 |
| 378 | 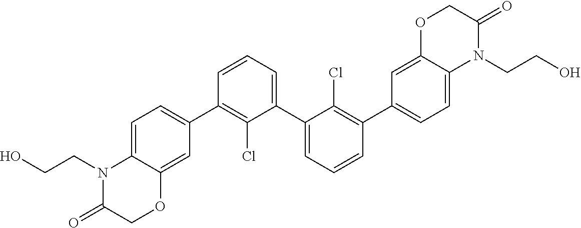 [SMILES: O=C1COc2cc(-c3cccc(-c4cccc(-c5ccc6c(c5)OCC(=O)N6CCO)c4Cl)c3Cl)ccc2N1CCO] | 605.50 |
| 384 | 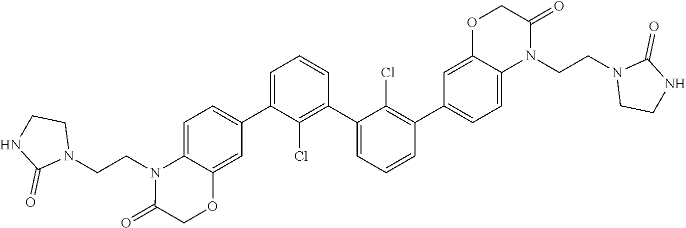 [SMILES: O=C1NCCN1CCN1C(=O)COc2cc(-c3cccc(-c4cccc(-c5ccc6c(c5)OCC(=O)N6CCN5CCNC5=O)c4Cl)c3Cl)ccc21] | 741.50 |
| 386 | 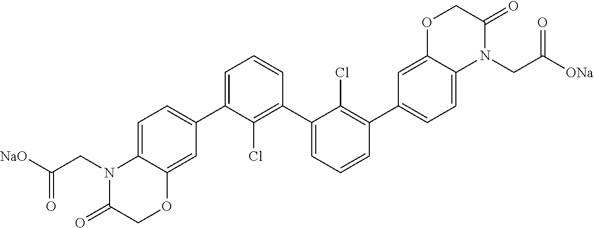 | 633.37 |
| 427 | 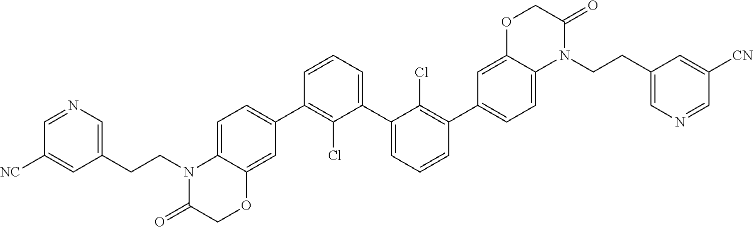 [SMILES: N#Cc1cncc(CCN2C(=O)COc3cc(-c4cccc(-c5cccc(-c6ccc7c(c6)OCC(=O)N7CCc6cncc(C#N)c6)c5Cl)c4Cl)ccc32)c1] | 777.45 |
| 394 | 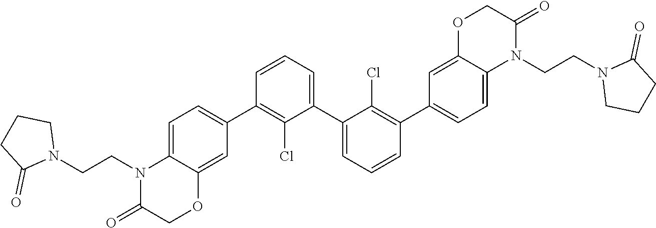 [SMILES: O=C1CCCN1CCN1C(=O)COc2cc(-c3cccc(-c4cccc(-c5ccc6c(c5)OCC(=O)N6CCN5CCCC5=O)c4Cl)c3Cl)ccc21] | 739.50 |
| 397 | 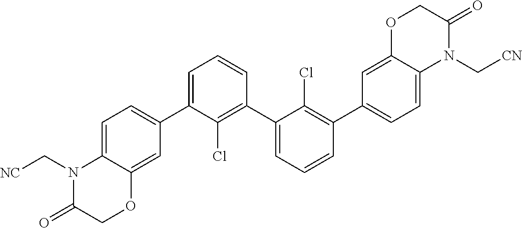 [SMILES: N#CCN1C(=O)COc2cc(-c3cccc(-c4cccc(-c5ccc6c(c5)OCC(=O)N6CC#N)c4Cl)c3Cl)ccc21] | 595.40 |
| 408 | 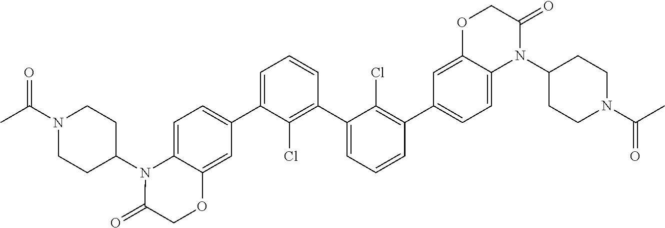 [SMILES: CC(=O)N1CCC(N2C(=O)COc3cc(-c4cccc(-c5cccc(-c6ccc7c(c6)OCC(=O)N7C6CCN(C(C)=O)CC6)c5Cl)c4Cl)ccc32)CC1] | 767.56 |
| 411 | 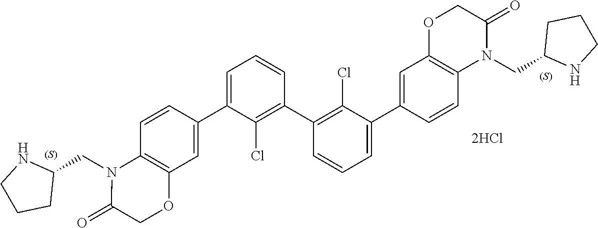 | 683.2 |
| 503 | 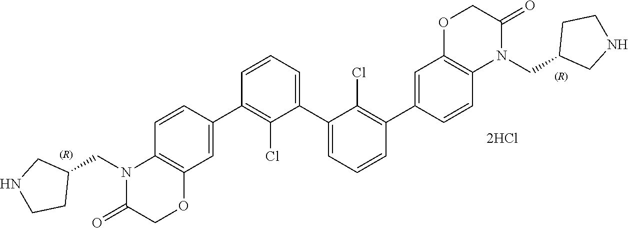 | 683.2 |
| 530 | 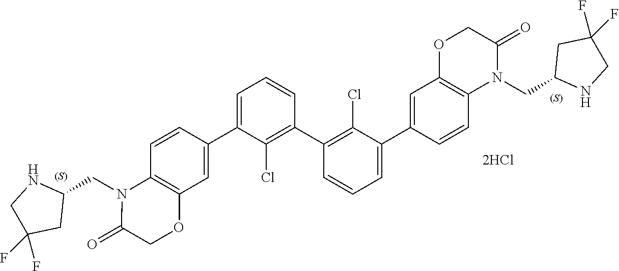 | 755.2 |
| 531 | 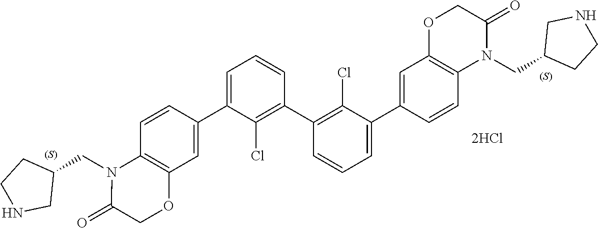 | 683.2 |
| 535 | 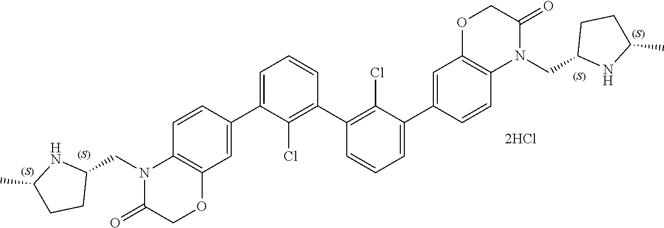 | 711.2 |
| 539 | 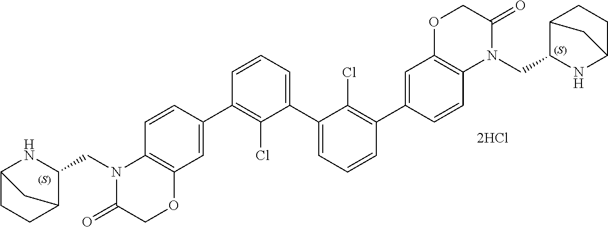 | 735.2 |
| 540 | 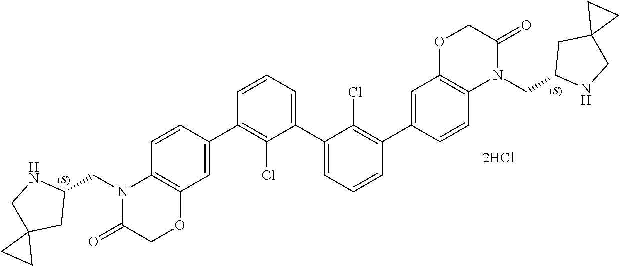 | 735.2 |
| 412 | 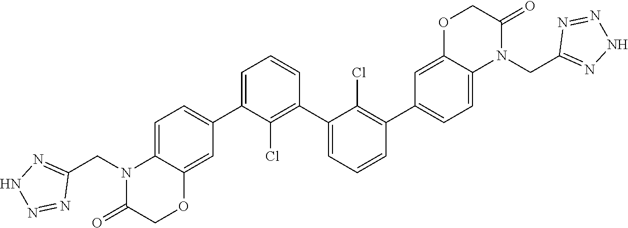 [SMILES: O=C1COc2cc(-c3cccc(-c4cccc(-c5ccc6c(c5)OCC(=O)N6Cc5nn[nH]n5)c4Cl)c3Cl)ccc2N1Cc1nn[nH]n1] | 681.41 |
| 413 | 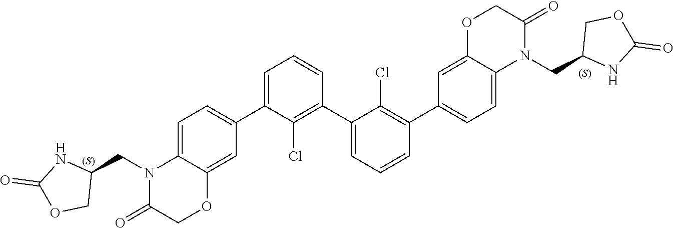 | 715.42 |
| 415 | 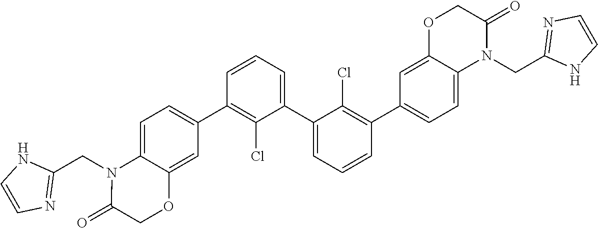 [SMILES: O=C1COc2cc(-c3cccc(-c4cccc(-c5ccc6c(c5)OCC(=O)N6Cc5ncc[nH]5)c4Cl)c3Cl)ccc2N1Cc1ncc[nH]1] | 677.43 |
| 459 | 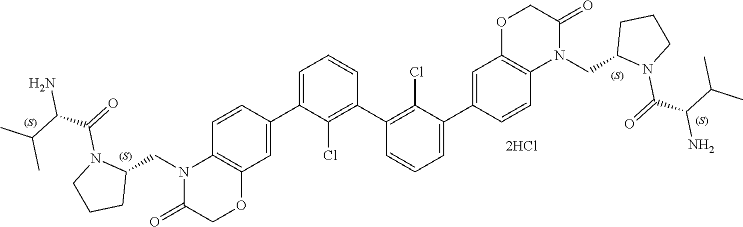 | 881.4 |
| 422 | 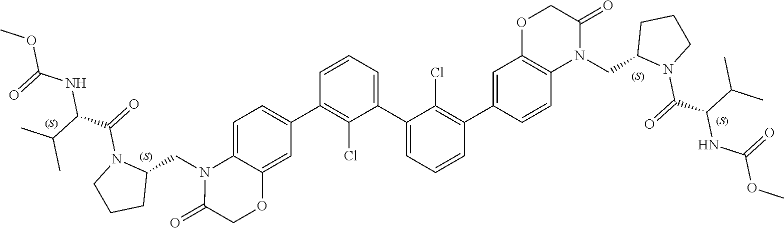 | 997.4 |
| 430 | 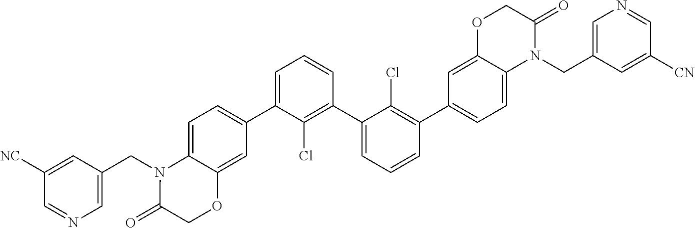 [SMILES: N#Cc1cncc(CN2C(=O)COc3cc(-c4cccc(-c5cccc(-c6ccc7c(c6)OCC(=O)N7Cc6cncc(C#N)c6)c5Cl)c4Cl)ccc32)c1] | 749.44 |
| 472 | 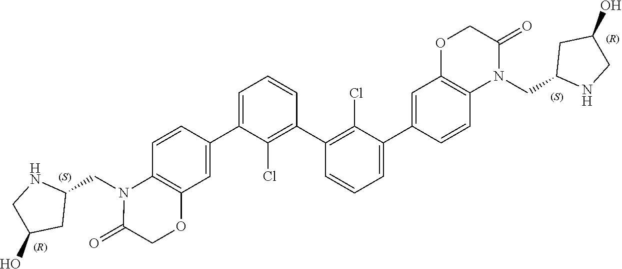 | 715.2 |
| 567 | 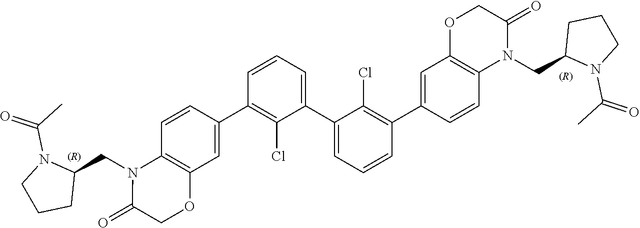 | 767.2 |
| 529 | 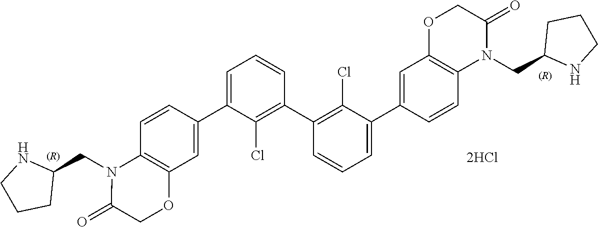 | 683.49 |
| 558 | 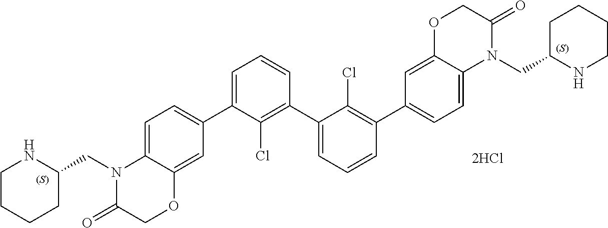 | 711.43 |
| 559 | 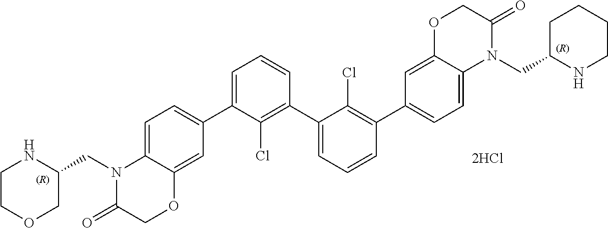 | 715.39 |
| 538 | 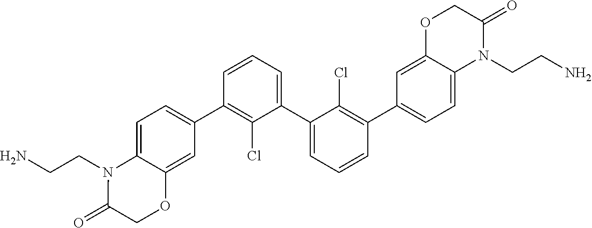 [SMILES: NCCN1C(=O)COc2cc(-c3cccc(-c4cccc(-c5ccc6c(c5)OCC(=O)N6CCN)c4Cl)c3Cl)ccc21] | 603.15 |
| 429 | 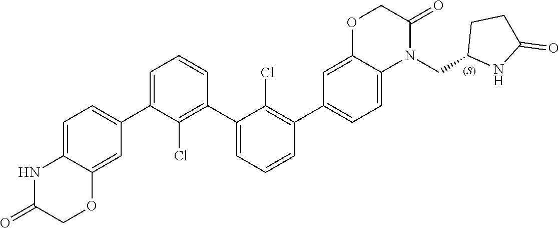 | 614.41 |
| 385 | 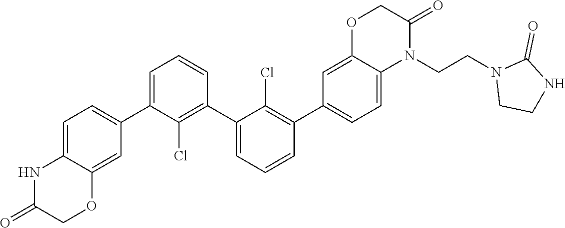 [SMILES: O=C1COc2cc(-c3cccc(-c4cccc(-c5ccc6c(c5)OCC(=O)N6CCN5CCNC5=O)c4Cl)c3Cl)ccc2N1] | 629.44 |
| 533 | 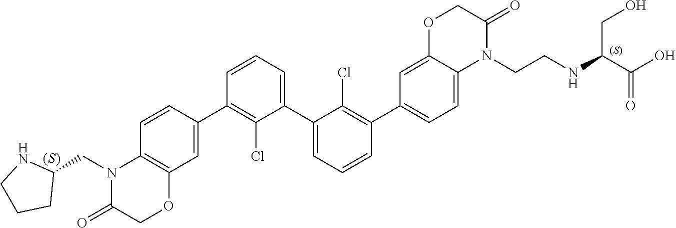 | 731.21 |
| 516 | 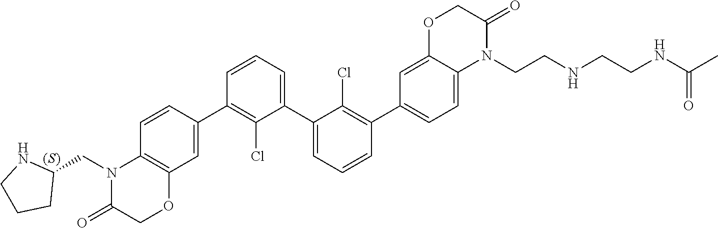 | 728.23 |
| 515 | 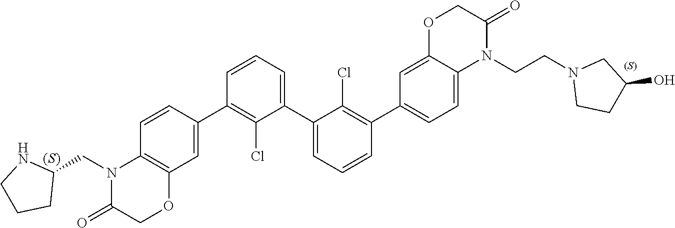 | 713.22 |
| 542 | 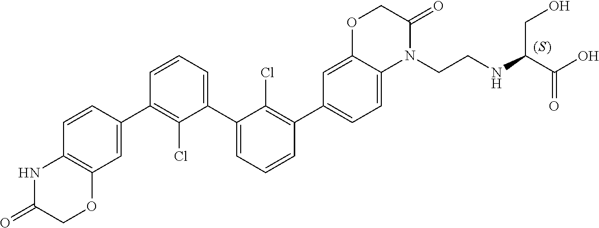 | 648.12 |
| 488 | 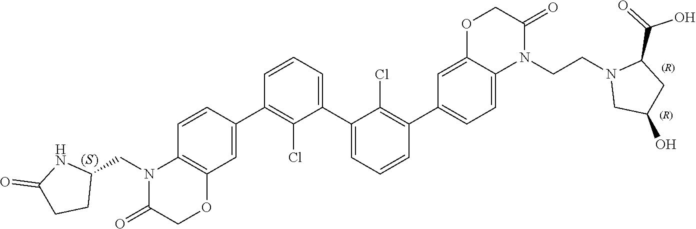 | 771.19 |
| 489 | 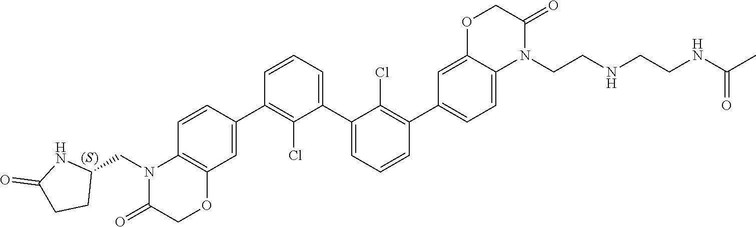 | 742.21 |
| 492 | 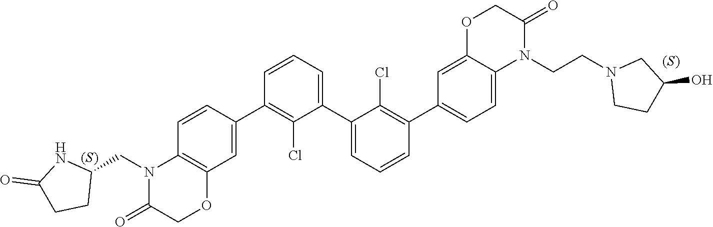 | 727.20 |
| 450 | 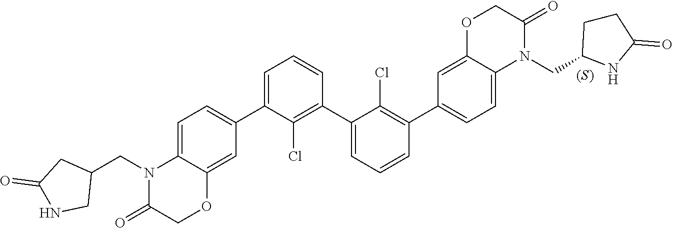 | 711.51 |
| 517 | 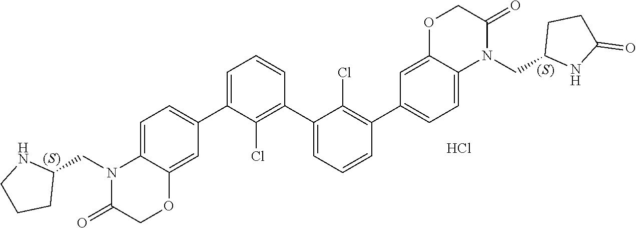 | 697.47 |

The present application further includes isotopically-substituted compounds of the disclosure. An “isotopically-substituted” compound is a compound of the application where one or more atoms are replaced or substituted by an atom having the same atomic number but a different atomic mass or mass number, e.g., a different atomic mass or mass number from the atomic mass or mass number typically found in nature (i.e., naturally occurring). It is to be understood that a “radio-labeled” compound is a compound that has incorporated at least one isotope that is radioactive (e.g., radionuclide).

### III. Uses of the Compound of Formula (I)

Another aspect of the present application relates the use of the compounds of formula (I). The compounds of formula (I) interfere with the interaction between PD-L1 and PD-1 and, thus, are useful in treating diseases and disorders associated with activity of PD-1 and the diseases and disorders associated with PD-L1.

In some embodiments, the compounds of formula (I) promote the formation of PD-L1 dimers and therefore, inhibits the interaction between PD-L1 and PD-1. In certain embodiments, the compounds of the present disclosure, or pharmaceutically acceptable salts or stereoisomers thereof, are useful for therapeutic administration to enhance, stimulate and/or increase immunity in cancer, chronic infection or sepsis, including enhancement of response to vaccination. In some embodiments, the present disclosure provides a method for inhibiting the PD-1/PD-L1 protein/protein interaction. The method includes administering to an individual or a patient an effective amount of the compound of formula (I), or a pharmaceutically acceptable salt or a stereoisomer thereof. The compounds of formula (I) can be used alone, in combination with other agents or therapies or as an adjuvant or neoadjuvant for the treatment of diseases or disorders, including cancer or infection diseases. For the uses described herein, any of the compounds of the disclosure, including any of the embodiments thereof, may be used.

The compounds of the present application inhibit the PD-1/PD-L1 protein/protein interaction, resulting in a PD-1 pathway blockade. The blockade of PD-1 can enhance the immune response to cancerous cells and infectious diseases in mammals, including humans. In some embodiments, the present disclosure provides treatment of an individual or a patient in vivo using a compound of any of the formulas herein or a salt or stereoisomer thereof such that growth of cancerous tumors is inhibited. A compound of any of the formulas as described herein, or a compound as recited in any of the claims and described herein, or a salt or stereoisomer thereof, can be used to inhibit the growth of cancerous tumors. Alternatively, a compound of any of the formulas as described herein, or a compound as recited in any of the claims and described herein, or a salt or stereoisomer thereof, can be used in conjunction with other agents or standard cancer treatments, as described below. In one embodiment, the present disclosure provides a method for inhibiting growth of tumor cells in vitro. The method includes contacting the tumor cells in vitro with a compound of any of the formulas as described herein, or of a compound as recited in any of the claims and described herein, or of a salt or stereoisomer thereof. In another embodiment, the present disclosure provides a method for inhibiting growth of tumor cells in an individual or a patient. The method includes administering to the individual or patient in need thereof a therapeutically effective amount of a compound of any of the formulas as described herein, or of a compound as recited in any of the claims and described herein, or a salt or a stereoisomer thereof.

In some embodiments, provided herein is a method for treating cancer. The method includes administering to a patient in need thereof, a therapeutically effective amount of any of the formulas as described herein, a compound as recited in any of the claims and described herein, or a salt thereof. Examples of cancers include those whose growth may be inhibited using compounds of the disclosure and cancers typically responsive to immunotherapy.

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers, especially metastatic cancers that express PD-L1.

In some embodiments, cancers treatable with compounds of the present disclosure include melanoma (e.g., metastatic malignant melanoma, cutaneous melanoma), renal cancer (e.g. clear cell carcinoma), prostate cancer (e.g. hormone refractory prostate adenocarcinoma), breast cancer (e.g., breast invasive carcinoma), colon cancer, lung cancer (e.g. non-small cell lung cancer and small cell lung cancer), squamous cell head and neck cancer (e.g., squamous cell carcinoma of the head and neck), urothelial cancer (e.g., bladder cancer, nonmuscle invasive bladder cancer (NMIBC)) and cancers with high microsatellite instability (MSIhlgh).

Additionally, the disclosure includes refractory or recurrent malignancies whose growth may be inhibited using the compounds of the disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CMIL), DLBCL, mantle cell lymphoma, Non-Hodgkin lymphoma (including relapsed or refractory NHL and recurrent follicular), Hodgkin lymphoma or multiple myeloma) and combinations of said cancers.

In some embodiments, cancers that are treatable using the compounds of the present disclosure include, but are not limited to, cholangiocarcinoma, bile duct cancer, biliary tract cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET)), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC) (e.g., squamous cell NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, and mesothelioma.

Exemplary gastrointestinal cancers include cancers of the esophagus (carcinoma, squamous cell carcinoma, adenocarcinoma, leiomyosarcoma, lymphoma), stomach (carcinoma, lymphoma, leiomyosarcoma, adenocarcinoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer (e.g., colorectal adenocarcinoma).

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma). In some embodiments, the cancer is a urological cancer (e.g., papilliary kidney carcinoma, testicular germ cell cancer, chromophobe renal cell carcinoma, clear cell renal carcinoma, or prostate adenocarcinoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors.

Exemplary nervous system cancers include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, serous adenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma (e.g., cutaneous squamous cell carcinoma), Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

PD-1 pathway blockade with compounds of the present disclosure can also be used for treating infections such as viral, bacteria, fungus and parasite infections. In some embodiments, provided herein is a method for treating infections. The method includes administering to a patient in need thereof, a therapeutically effective amount of any of the formulas as described herein, a compound as recited in any of the claims and described herein, a salt thereof. Examples of viruses causing infections treatable by methods of the present disclosure include, but are not limit to, human immunodeficiency virus, human papillomavirus, influenza, hepatitis A, B, C or D viruses, adenovirus, poxvirus, herpes simplex viruses, human cytomegalovirus, coronaviruses, severe acute respiratory syndrome virus, ebola virus, and measles virus. In some embodiments, viruses causing infections treatable by methods of the present disclosure include, but are not limit to, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, cornovirus, respiratory syncytial virus, mumpsvirus, rotavirus, measles virus, rubella virus, parvovirus, vaccinia virus, HTLV virus, dengue virus, papillomavirus, molluscum virus, poliovirus, rabies virus, JC virus, tuberculosis and arboviral encephalitis virus.

The present disclosure provides a method for treating bacterial infections. The method includes administering to a patient in need thereof, a therapeutically effective amount of a compound of any of the formulas as described herein, a compound as recited in any of the claims and described herein, or a salt thereof. Non-limiting examples of pathogenic bacteria causing infections treatable by methods of the disclosure include chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

The present disclosure provides a method for treating fungus infections. The method includes administering to a patient in need thereof, a therapeutically effective amount of a compound of any of the formulas as described herein, a compound as recited in any of the claims and described herein, or a salt thereof. Non-limiting examples of pathogenic fungi causing infections treatable by methods of the disclosure include Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Flistoplasma capsulatum.

The present disclosure provides a method for treating parasite infections. The method includes administering to a patient in need thereof, a therapeutically effective amount of a compound of any of the formulas as described herein, a compound as recited in any of the claims and described herein, or a salt thereof. Non-limiting examples of pathogenic parasites causing infections treatable by methods of the disclosure include Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

In some embodiments, provided herein is a method for treating inflammation. The method includes administering to a patient in need thereof, a therapeutically effective amount of a compound of any of the formulas as described herein, a compound as recited in any of the claims and described herein, or a salt thereof.

In some embodiments, provided herein is a method for treating an autoimmune disease. The method includes administering to a patient in need thereof, a therapeutically effective amount of a compound of any of the formulas as described herein, a compound as recited in any of the claims and described herein, or a salt thereof.

It is believed that compounds of formula (I), or any of the embodiments thereof, may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

In some embodiments, the compounds of the application are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

In some embodiments, the present disclosure provides a method of enhancing, stimulating and/or increasing the immune response in a patient. The method includes administering to the patient in need thereof a therapeutically effective amount of any of the formulas as described herein, a compound or composition as recited in any of the claims and described herein, or a salt thereof.

### Combination Therapies

The compounds of the present disclosure can be used in combination with one or more other therapies for the treatment of diseases, such as cancer or infections. Examples of diseases and indications treatable with combination therapies include those as described herein.

Examples of cancers include solid tumors and non-solid tumors, such as liquid tumors, blood cancers. Examples of infections include viral infections, bacterial infections, fungus infections or parasite infections. For example, the compounds of the present disclosure can be combined with one or more inhibitors of the following kinases for the treatment of cancer: Akt1, Akt2, Akt3, BCL2, CDK, TGF-PR, PKA, PKG, PKC, CaM-kinase, phosphorylase kinase, MEKK, ERK, MAPK, mTOR, EGFR, HER2, HER3, HER4, INS—R, IDH2, IGF-1R, IR—R, PDGFotR, PDGi'PR, PI3K (alpha, beta, gamma, delta, and multiple or selective), CSF1R, KIT, FLK-1I, KDR/FLK-1, FLK-4, fit-1, FGFR1, FGFR2, FGFR3, FGFR4, c-Met, PARP, Ron, Sea, I RKA, TRKB, TRKC, TAM kinases (Axl, Mer, Tyro3), FLT3, VEGFR/FH2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the compounds of the present disclosure can be combined with one or more of the following inhibitors for the treatment of cancer or infections. Non-limiting examples of inhibitors that can be combined with the compounds of the present disclosure for treatment of cancer and infections include an FGFR inhibitor (FGFR1, FGFR2, FGFR3 or FGFR4, e.g., pemigatinib (INCY54828), INCB62079), a JAK inhibitor (JAK1 and/or JAK2, e.g., ruxolitinib, baricitinib or itacitinib (INCB39110)), an IDO inhibitor (e.g., epacadostat, NLG919, or BMS-986205, MK7162), an LSD1 inhibitor (e.g., INCB59872 and INCB60003), a TDO inhibitor, a PI3K-delta inhibitor (e.g., Parsaclisib (INCB50465) and INCB50797), a PI3K-gamma inhibitor such as PI3K-gamma selective inhibitor, a Pirn inhibitor (e.g., INCB53914), an EGFR inhibitor (also known as ErB-1 or HER-1; e.g. erlotinib, gefitinib, vandetanib, orsimertinib, cetuximab, necitumumab, or panitumumab), a VEGFR inhibitor or pathway blocker (e.g. bevacizumab, pazopanib, sunitinib, sorafenib, axitinib, regorafenib, ponatinib, cabozantinib, axitinib, vandetanib, ramucirumab, lenvatinib, ziv-aflibercept), a PARP inhibitor (e.g. olaparib, rucaparib, veliparib, talazoparib, or niraparib), a CSF1R inhibitor, a TAM receptor tyrosine kinases (Tyro-3, Axl, and Mer), an adenosine receptor antagonist (e.g., A2a/A2b receptor antagonist), an HPK1 inhibitor, a chemokine receptor inhibitor (e.g. CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), an arginase inhibitor (INCB001158), a PARP inhibitor (such as rucaparib or olaparib), sitravatinib, a B-Raf inhibitor-MEK inhibitor combination (such as encorafenib plus binimetinib, dabrafenib plus trametinib, or cobimetinib plus vemurafenib), and an adenosine receptor antagonist or combinations thereof.

In some embodiments, the compounds of the present disclosure can be combined with a TLR7 agonist (e.g., imiquimod).

The compounds of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, STING agonists, RIG-I agonists, oncolytic virotherapy and immunomodulating small molecules, including thalidomide or JAK1/2 inhibitor, PI3K6 inhibitor and the like. The compounds can be administered in combination with one or more anti-cancer drugs, such as a chemotherapeutic agent. Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paelitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat and zoledronate.

Other anti-cancer agent(s) include antibody therapeutics such as trastuzumab (Herceptin), antibodies to costimulatory molecules such as CTLA-4 (e.g., ipilimumab), 4-1BB (e.g. urelumab, utomilumab), antibodies to PD-1 and PD-L1, or antibodies to cytokines (IL-10, TGF-b, etc.). Examples of antibodies to PD-1 and/or PD-L1 that can be combined with compounds of the present disclosure for the treatment of cancer or infections such as viral, bacteria, fungus and parasite infections include, but are not limited to nivolumab, pembrolizumab, atezolizumab, durvalumab, avelumab and SHR-1210.

Compounds of the present disclosure can be used in combination with one or more immune checkpoint inhibitors for the treatment of diseases, such as cancer or infections.

Exemplary immune checkpoint inhibitors include inhibitors against immune checkpoint molecules such as CBL-B, CD27, CD28, CD40, CD122, CD96, CD73, CD47, 0X40, GITR, CSF1R, JAK, PI3K delta, PI3K gamma, TAM, arginase, CD137 (also known as 4-1BB), ICOS, A2AR, B7-113, B7-114, BTLA, CTLA-4, LAG3, TIM3, TIGIT, CD112R, VISTA, PD-1, PD-L1 and PD-L2. In some embodiments, the immune checkpoint molecule is a stimulatory checkpoint molecule selected from CD27, CD28, CD40, ICOS, 0X40, GITR and CD137. In some embodiments, the immune checkpoint molecule is an inhibitory checkpoint molecule selected from A2AR, B7-113, B7-114, BTLA, CTLA-4, IDO, KIR, LAG3, PD-1, TIM3, and VISTA. In some embodiments, the compounds provided herein can be used in combination with one or more agents selected from KIR inhibitors, TIGIT inhibitors, LAIR1 inhibitors, CD 160 inhibitors, 2B4 inhibitors and TGFR beta inhibitors.

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP-224. In some embodiments, the anti-PD-1 monoclonal antibody is nivolumab or pembrolizumab. In some embodiments, the anti-PD1 antibody is pembrolizumab.

In some embodiments, the inhibitor of an immune checkpoint molecule is an inhibitor of CTLA-4, e.g., an anti-CTLA-4 antibody. In some embodiments, the anti-CTLA-4 antibody is ipilimumab or tremelimumab.

In some embodiments, the inhibitor of an immune checkpoint molecule is an inhibitor of LAG3, e.g., an anti-LAG3 antibody. In some embodiments, the anti-LAG3 antibody is BMS-986016, LAG525 or INCAGN2385.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of GITR, e.g., an anti-GITR antibody. In some embodiments, the anti-GITR antibody is TRX518, MK-4166, INCAGN1876, MK-1248, AMG228, BMS-986156, GWN323, or MEDI1873.

In some embodiments, the inhibitor of an immune checkpoint molecule is an inhibitor of 0X40, e.g., an anti-OX40 antibody or OX40L fusion protein. In some embodiments, the anti-0X40 antibody is MEDI0562, MOXR-0916, PF-04518600, GSK3174998, or BMS-986178. In some embodiments, the OX40L fusion protein is MEDI6383.

The compounds of the present disclosure can further be used in combination with one or more anti-inflammatory agents, steroids, immunosuppressants or therapeutic antibodies.

The compounds of any of the formulas as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gplOO, MAGE antigens, Trp-2, MARTI and/or tyrosinase, or tumor cells transfected to express the cytokine GM-CSF.

The compounds of any of the formulas as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Vims (KHSV). In some embodiments, the compounds of the present disclosure can be used in combination with tumor specific antigen such as heat shock proteins isolated from tumor tissue itself. In some embodiments, the compounds of any of the formulas as described herein, a compound as recited in any of the claims and described herein, or salts thereof can be combined with dendritic cells immunization to activate potent anti-tumor responses.

The compounds of the present disclosure can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

The compounds of the present disclosure can be used in combination with bone marrow transplant for the treatment of a variety of tumors of hematopoietic origin.

The compounds of any of the formulas as described herein, a compound as recited in any of the claims and described herein, or salts thereof can be used in combination with vaccines, to stimulate the immune response to pathogens, toxins, and self antigens.

When more than one pharmaceutical agent is administered to a patient, they can be administered simultaneously, separately, sequentially, or in combination (e.g., for more than two agents).

### Formulation, Dosage Forms and Route of Administration

When employed as pharmaceuticals, the compounds of the present disclosure can be administered in the form of pharmaceutical compositions. Thus the present disclosure provides a composition comprising a compound of any of the formulas as described herein, a compound as recited in any of the claims and described herein, or a pharmaceutically acceptable salt thereof, or any of the embodiments thereof, and at least one pharmaceutically acceptable carrier or excipient. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary {e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration.

Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This application also includes pharmaceutical compositions which contain, as the active ingredient, the compound of the present disclosure or a pharmaceutically acceptable salt thereof, in combination with one or more pharmaceutically acceptable carriers or excipients. In some embodiments, the composition is suitable for topical administration. In making the compositions of the application, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the application may be milled using known milling procedures such as wet milling to obtain a particle size appropriate for tablet formation and for other formulation types. Finely divided (nanoparticulate) preparations of the compounds of the application can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the application can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel K00LV™). In some embodiments, the polyethylene oxide is polyethylene oxide WSR 1105 (e.g, Poly ox WSR 1105™).

In some embodiments, a wet granulation process is used to produce the composition. In some embodiments, a dry granulation process is used to produce the composition.

The compositions can be formulated in a unit dosage form, each dosage containing from about 5 to about 1,000 mg (1 g), more usually about 100 mg to about 500 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g, at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present application can vary according to, e.g., the particular use for which the treatment is made, the manner of administration of the compound, the health and condition of the patient, and the judgment of the prescribing physician. The proportion or concentration of a compound of the application in a pharmaceutical composition can vary depending upon a number of factors including dosage, chemical characteristics (e.g., hydrophobicity), and the route of administration. For example, the compounds of the application can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid preformulation composition containing a homogeneous mixture of a compound of the present application. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, e.g., about 0.1 to about 1000 mg of the active ingredient of the present application.

The tablets or pills of the present application can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds and compositions of the present application can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxy ethyl cellulose, and the like. In some embodiments, topical formulations contain at least about 0.1, at least about 0.25, at least about 0.5, at least about 1, at least about 2 or at least about 5 wt % of the compound of the application. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers or stabilizers will result in the formation of pharmaceutical salts.

The therapeutic dosage of a compound of the present application can vary according to, e.g., the particular use for which the treatment is made, the manner of administration of the compound, the health and condition of the patient, and the judgment of the prescribing physician. The proportion or concentration of a compound of the application in a pharmaceutical composition can vary depending upon a number of factors including dosage, chemical characteristics (e.g., hydrophobicity), and the route of administration. For example, the compounds of the application can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The present application is further illustrated by the following examples that should not be construed as limiting. The contents of all references, patents, and published patent applications cited throughout this application, as well as the Figures and Tables, are incorporated herein by reference.

### EXAMPLES

### Example 1

### Preparation of (S)-(5-oxopyrrolidin-2-yl)methyl 4-methylbenzenesulfonate (Intermediate SM1)

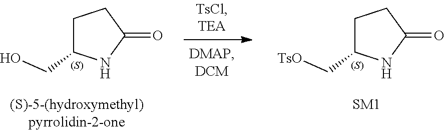

A solution of (S)-5-(hydroxymethyl)pyrrolidin-2-one (0.100 g, 0.869 mmol, 1 eq), TsCl (0.182 g, 0.954 mmol, 1.10 eq), TEA (0.132 g, 1.306 mmol, 1.5 eq), DMAP (0.006 g, 0.049 mmol, 0.05 eq) in DCM (2 ml) was stirred at rt for 16 h, IN HCl (5 ml) was slowly added, extracted with DCM, the organic was concentrated to give SM1 (0.170 g, 73%).

### Example 2

### Preparation of 2,2′-(2,2′-dichloro-[1,1′-biphenyl]-3,3′-diyl)bis(4,4,5,5-tetramethyl-1,3,2-dioxaborolane) (Intermediate SM2)

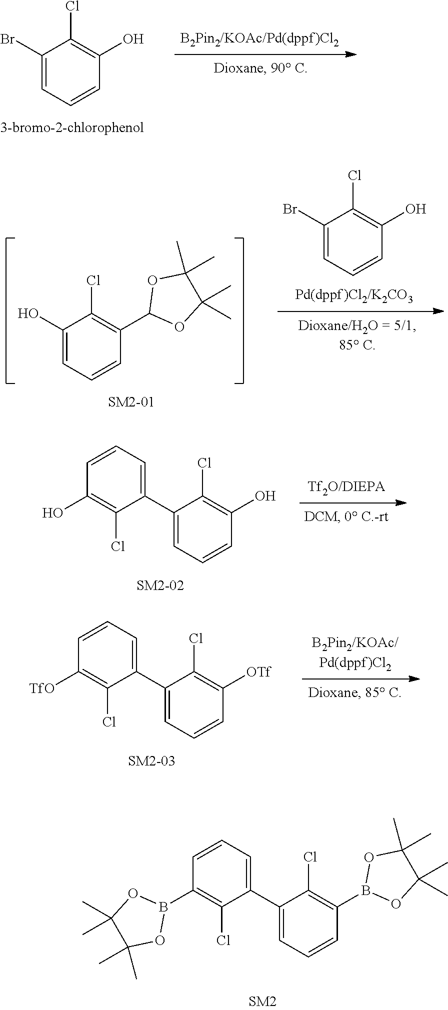

### Step 1 and 2

To a 500 ml round-bottom flask were added 3-bromo-2-chlorophenol (12.4 g, 0.060 mol, 1.0 eq), B₂Pin₂ (16.4 g, 0.065 mol, 1.08 eq), KOAc (20.5 g, 0.210 mol, 3.5 eq), Pd (dppf) Cl₂·DCM (4.1 g, 5.1 mmol, 0.085 eq), followed by added the solvent dioxane (300 ml), the final mixture was charged with N2 and stirred at 95° C. for 3 h. Then the reaction mixture was cooled down to room temperature, filtered, the filter cake was washed with dioxane (100 ml), the filtrate was used directly in the next step.

To the previous filtrate were added 3-bromo-2-chlorophenol (12.0 g, 0.059 mol, 0.99 eq), K₂CO₃ (24.8 g, 0.180 mol, 3.0 eq) and Pd(dppf)Cl2·DCM (2.1 g, 2.40 mmol, 0.042 eq), followed by added H2O (80 ml), the final mixture was charged with N2 and stirred at 85° C. for 3.5 h. Then the reaction mixture was cooled down to room temperature and filtered, the filter cake was washed with EA (300 ml). The brine (300 ml) was added to the filtrate and separated, the aqueous phase was extracted with EA (100 ml×2), The combined organic phase was discolored with activated carbon at room temperature overnight. The mixture was filtered through a pad of celite, the filter cake was washed with EA, the combined organic phase was concentrated under vacuum. The residue was purified by recrystallization from DCM/PE=1.5/1 to afford desired product (10.1 g, yield: 46%) as a light yellow solid.

### Step 3

To a stirred mixture of SM2-02 (10.1 g, 0.039 mol, 1.0 eq) in DCM (200 ml) was added DIPEA (19.4 g, 0.151 mol, 3.8 eq) at 0° C., after the SM2-02 was dissolved, followed by added Tf2O (26.8 g, 0.095 mol, 2.4 eq) at this temperature, then allowed to room temperature for stirring another 2 h. The water (100 ml) was added to work-up the reaction, then separated, the aqueous phase was extracted with DCM (100 ml). The combined organic phase was washed with brine (200 ml), dried over anhydrous sodium sulfate, filtered, the filtrate was concentrated under vacuum. The residue was purified by recrystallized with EtOH/H₂O=1/1 to afford desired product (18.3 g, yield 89%) as a light yellow solid.

### Step 4

To a stirred solution of SM2-03 (14.2 g, 0.027 mol, 1.0 eq) in dioxane (80 ml) were added B₂Pin₂ (27.8 g, 0.109 mol, 4.0 eq), KOAc (16 g, 0.164 mol, 6.0 eq) and Pd (dppf) Cl₂·DCM (3.3 g, 4.1 mmol, 0.15 eq.) at room temperature, then charged with N2 and stirred at 85° C. for 2 h. Then cooled downed to room temperature, EA (150 ml) and water (150 ml) were added to the mixture, separated, the aqueous phase was extracted with EA (100 ml). The combined organic phase was washed with brine (200 ml), dried over anhydrous sodium sulfate, filtered, the filtrate was concentrated under vacuum. The residue was dissolved with EA (50 ml), then PE (300 ml) was added slowly to the solution to form a black suspension. After stirring for 30 min, filtered, the filter cake was washed with 140 ml (PE/EA=6/1), the filtrate was concentrated under vacuum. The residue was recrystallized with EtOH (150 ml) to afford desired product (9.5 g, yield: 75%) as an off-white solid.

### Example 3

### Preparation of 2,2′-((2,2′-dichloro-[1,1′-biphenyl]-3,3′-diyl)bis(3-oxo-2,3-dihydro-4H-benzo[b][1,4]oxazine-7,4-diyl))diacetaldehyde (Intermediate SM3)

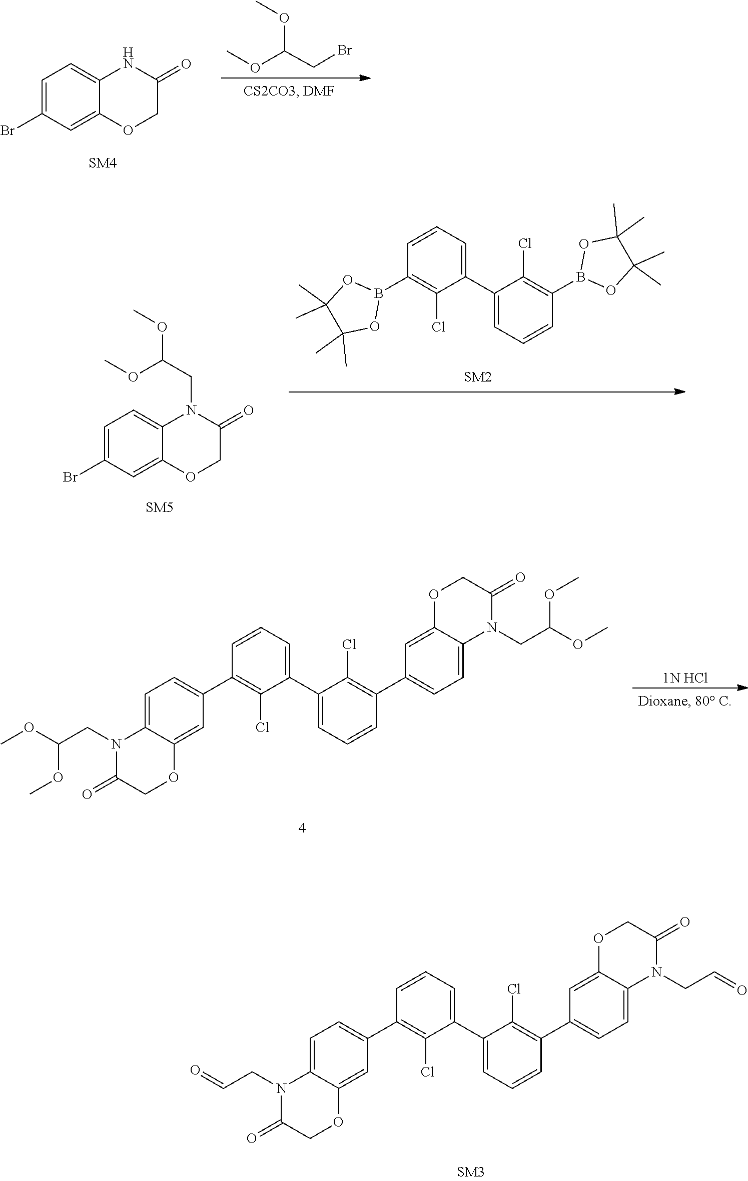

### Step 1

A solution of 7-bromo-2H-benzo[b][1,4]oxazin-3(4H)-one (5.00 g, 21.93 mmol, 1 eq), bromoacetaldehyde dimethyl acetal (5.55 g, 32.89 mmol, 1.5 eq), Cs₂CO₃ (14.29 g, 43.86 mmol, 2.0 eq) in DMF (60 ml) was stirred at 60° C. for 16 h, 180 mL H₂O was added, extracted by EA, the organic layer was collected and purified by silica gel to give 7-bromo-4-(2,2-dimethoxyethyl)-2H-benzo[b][1,4]oxazin-3(4H)-one (5.40 g, 78%).

### Step 2

A solution of compound SM2 (2.50 g, 5.26 mmol, 1 eq), 7-bromo-4-(2,2-dimethoxyethyl)-2H-benzo[b][1,4]oxazin-3(4H)-one (3.49 g, 11.04 mmol, 2.1 eq), K2CO3 (2.90 g, 21.04 mmol, 4.0 eq), Pd(dppf)Cl2·DCM (0.21 g, 0.263 mmol, 0.05 eq) in dioxane (40 ml) and H2O (8 ml) was stirred at 80° C. for 2 h under N₂, 30 mL H2O was added, extracted by EA, the organic layer was collected and purified by silica gel to give 7,7′-(2,2′-dichloro-[1,1′-biphenyl]-3,3′-diyl)bis(4-(2,2-dimethoxyethyl)-2H-benzo[b][1,4]oxazin-3(4H)-one) (3.10 g, 85%)

### Step 3

A solution of 7,7′-(2,2′-dichloro-[1,1′-biphenyl]-3,3′-diyl)bis(4-(2,2-dimethoxyethyl)-2H-benzo[b][1,4]oxazin-3(4H)-one) (3.10 g, 4.47 mmol, 1 eq) of 1N HCl aq/dioxane (15 ml/30 ml) was stirred at 80° C. for 1 h, 30 mL H₂O was added, extracted by EA, the organic layer was washed with NaHCO₃ and concentrated to give the title compound SM3. (2.91 g, crude).

### Preparation of 3,3′-((2,2′-dichloro-[1,1′-biphenyl]-3,3′-diyl)bis(3-oxo-2,3-dihydro-4H-benzo[b][1,4]oxazine-7,4-diyl))dipropanal (Intermediate SM4)

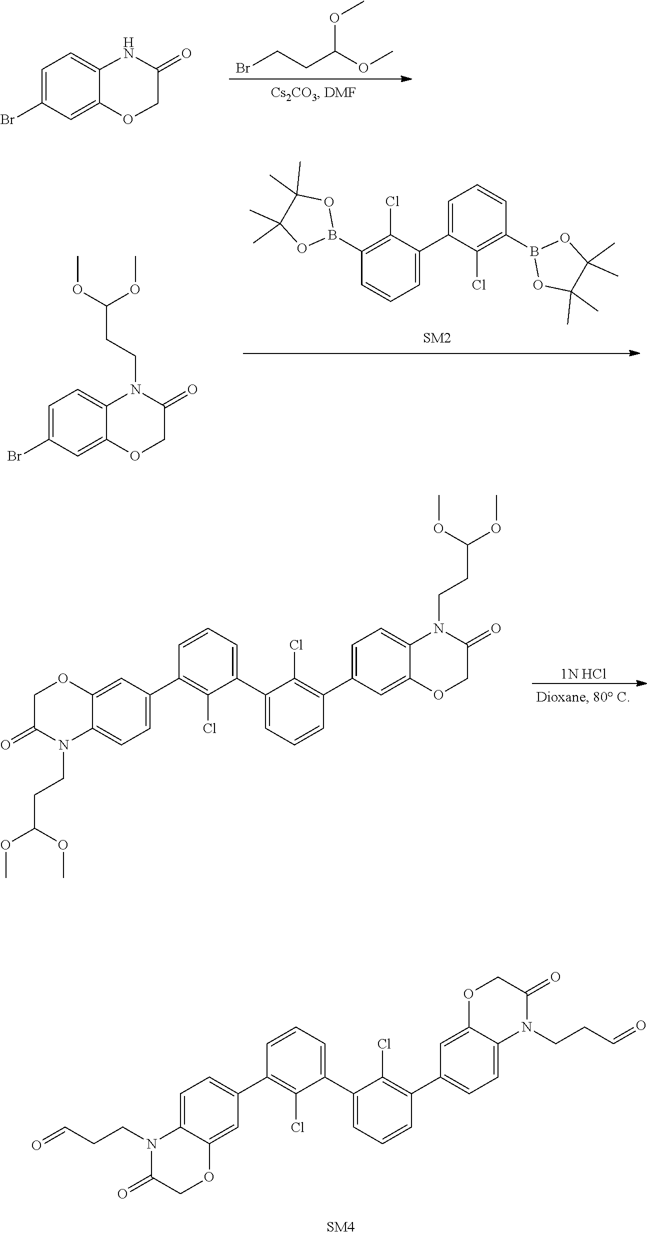

The compound SM4 can be prepared following the same procedure for SM3 using 3-bromo-1,1-dimethoxypropane as the reactant.

### Example 4

### Preparation of 7,7′-(2,2′-dichloro-[1,1′-biphenyl]-3,3′-diyl)bis(2H-benzo[b][1,4]oxazin-3(4H)-one) (Intermediate SM5)

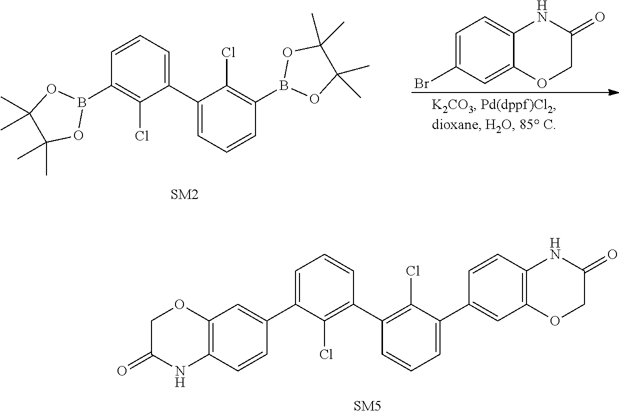

Referring to the following reaction equation, SM2 (0.05 g, 0.11 mmol, 1 eq.), 7-bromo-2H-benzo[b][1,4]oxazin-3(4H)-one (0.062 g, 0.27 mmol, 2.5 eq.), Pd(dppf)Cl2 (0.008 g, 0.01 mmol, 0.1 eq.) and potassium carbonate (0.058 g, 0.42 mmol, 4 eq.) were dissolved in Dioxane/H2O (3 ml, v/v=5:1). The reaction was carried out at 85° C. for 2 h under N₂ atmosphere. After cooling, 10 ml water and 10 ml EA were added for extraction, and the organic phase was concentrated and purified by Prep-TLC (DCM/MeOH=10/1 elution) to give Compound SM5, 7,7′-(2,2′-dichloro-[1,1′-biphenyl]-3,3′-diyl)bis(2H-benzo[b][1,4]oxazin-3(4H)-on e) (13 mg, yield: 15.6%).

### Example 5 (Preparation of Reductive Amination)

### 5A. Preparation of 1-(2-(7-(2,2′-dichloro-3′-(4-(2-((S)-3-hydroxypyrrolidin-1-yl)ethyl)-3-oxo-3,4-dihydro-2H-benzo[b][1,4]oxazin-7-yl)-[1,1′-biphenyl]-3-yl)-3-oxo-2,3-dihydro-4H-benzo[b][1,4]oxazin-4-yl)ethyl)pyrrolidine-3-carboxylic acid

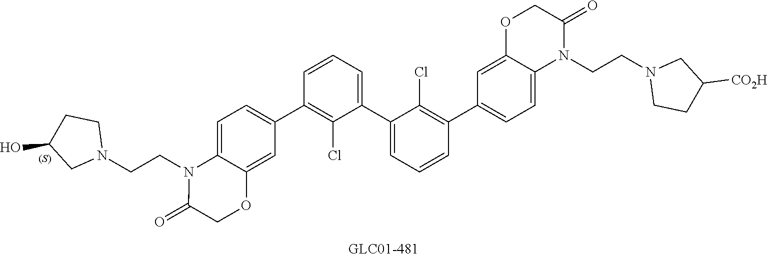

To a stirred solution of compound SM3 (0.015 g, 0.024 mmol, 1 eq), pyrrolidine-3-carboxylic acid (0.0035 g, 0.03 mmol, 1.3 eq), (S)-pyrrolidin-3-ol hydrochloride (0.004 g, 0.032 mmol, 1.3 eq) and one drop of AcOH in CH₂Cl₂/MeOH (1 mL/0.5 mL) at room temperature, sodium triacetoxyborohydride (0.051 g, 0.24 mmol, 10 eq) was added. After 4 h, concentrated directly and 0.5 mL H₂O and 3 mL MeOH was added, the mixture was purified by reverse phase HPLC (0.1% trifluoroacetic acid in water/acetonitrile) providing GLC01-481 (6 mg, 31%).

¹H NMR (500 MHz, DMSO-d₆) δ 7.52 (t, J=7.6 Hz, 2H), 7.46 (d, J=7.8 Hz, 2H), 7.41 (d, J=7.5 Hz, 2H), 7.36 (d, J=8.4 Hz, 2H), 7.19 (d, 2H), 7.14 (d, J=2.0 Hz, 2H), 5.53 (s, 2H), 4.75 (s, 4H), 4.49-4.43 (m, 2H), 4.34-4.26 (m, 4H), 3.75-3.67 (m, 4H), 3.24-3.17 (m, 4H), 2.34-2.16 (m, 4H), 1.57-1.35 (m, 4H).

LCMS (ESI): calculated for C₄₁H₄₀Cl₂N₄O₇; [M+H]⁺: 771.23, found: 771.50.

### 5B. The Following Compounds could be Prepared Using Different Amine Substrate with SM3 or SM4

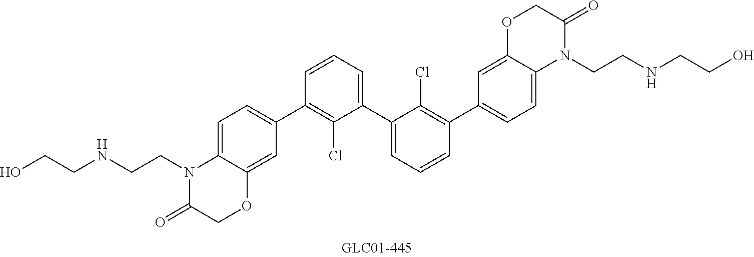 [SMILES: O=C1COc2cc(-c3cccc(-c4cccc(-c5ccc6c(c5)OCC(=O)N6CCNCCO)c4Cl)c3Cl)ccc2N1CCNCCO]

¹H NMR (500 MHz, DMSO-d₆) δ 7.52 (t, J=7.5 Hz, 2H), 7.45 (d, J=7.5 Hz, 2H), 7.41 (d, J=7.5 Hz, 2H), 7.36 (d, J=8.4 Hz, 2H), 7.18 (d, J=8.3 Hz, 2H), 7.14 (s, 2H), 4.75 (s, 4H), 4.29 (s, 4H), 3.66 (s, 2H) 3.35-3.29 (m, 8H), 2.29-2.20 (m, 8H).

LCMS (ESI): calculated for C₃₆H₃₆Cl₂N₄O₆; [M+H]⁺: 691.2, found: 691.2.

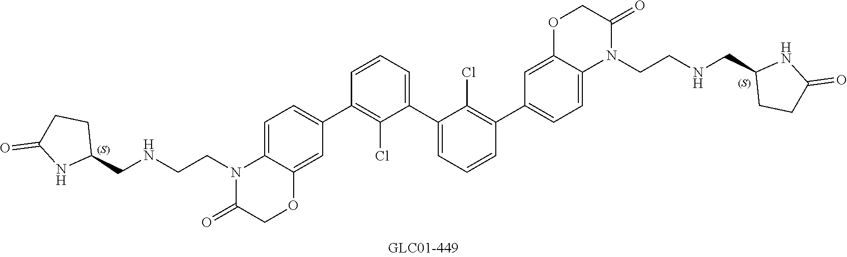

¹H NMR (500 MHz, DMSO-d₆) δ 7.62 (s, 2H), 7.52-7.44 (m, 5H), 7.38 (dd, J=17.4 Hz, 7.9 Hz, 4H), 7.16 (d, J=8.4 Hz, 2H), 7.10 (s, 2H), 4.70 (s, 4H), 3.99 (td, J=14.8 Hz, 7.5 Hz, 4H), 3.55 (dd, J=12.1 Hz, 6.0 Hz, 2H), 2.76 (s, 4H), 2.55 (d, J=5.7 Hz, 4H), 2.15-1.96 (m, 8H), 1.71-1.61 (m, 2H).

LCMS (ESI): calculated for C₄₂H₄₂Cl₂N₄O₆; [M+H]⁺: 797.3, found: 797.3.

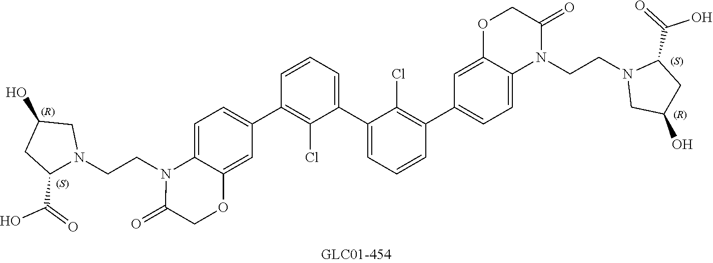

¹H NMR (500 MHz, DMSO-d₆) δ 7.51 (t, J=7.5 Hz, 2H), 7.46 (dd, J=7.6, 1.8 Hz, 2H), 7.40 (d, J=7.3 Hz, 2H), 7.35 (d, J=8.4 Hz, 2H), 7.19-7.15 (m, 2H), 7.13 (d, J=2.0 Hz, 2H), 4.73 (s, 4H), 4.36 (s, 2H), 4.31-4.14 (m, 6H), 3.72-3.64 (m, 4H), 3.12-2.97 (m, 4H), 2.20-2.08 (m, 4H).

LCMS (ESI): calculated for C₄₂H₄₀Cl₂N₄O₁₀; [M+H]⁺: 831.22, found: 831.45.

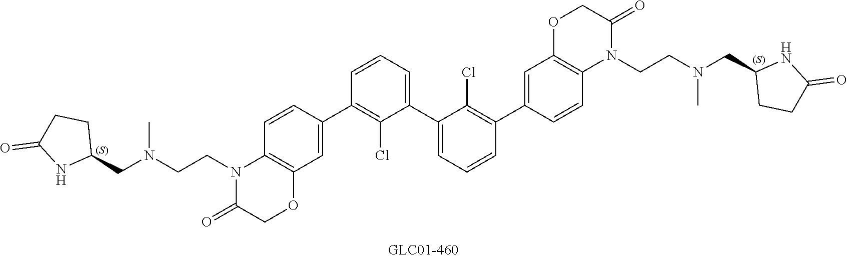

¹H NMR (500 MHz, DMSO-d₆) δ 7.48 (dt, J=13.7, 7.5 Hz, 6H), 7.40 (d, J=7.4 Hz, 2H), 7.33 (d, J=8.3 Hz, 2H), 7.17 (d, J=8.5 Hz, 2H), 7.11 (s, 2H), 4.69 (s, 4H), 4.08 (s, 2H), 4.02 (s, 2H), 3.50 (s, 2H), 2.63 (s, 2H), 2.58 (d, J=6.6 Hz, 4H), 2.27 (s, 6H), 2.14-1.92 (m, 8H), 1.65-1.57 (m, 2H).

LCMS (ESI): calculated for C₄₄H₄₆Cl₂N₆O₆; [M+H]⁺: 825.3, found: 825.3.

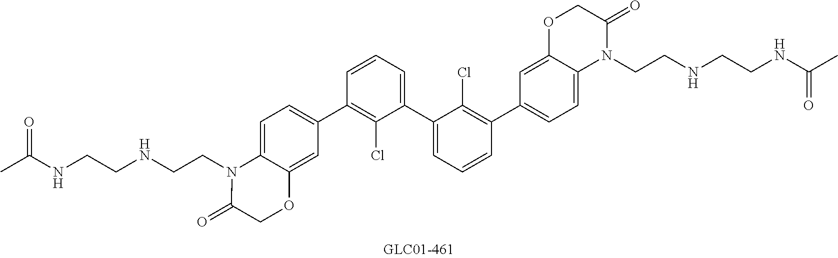 [SMILES: CC(=O)NCCNCCN1C(=O)COc2cc(-c3cccc(-c4cccc(-c5ccc6c(c5)OCC(=O)N6CCNCCNC(C)=O)c4Cl)c3Cl)ccc21]

¹H NMR (500 MHz, DMSO-d₆) δ 8.18 (t, J=5.8 Hz, 2H), 7.52 (t, J=7.6 Hz, 2H), 7.45 (d, J=7.6 Hz, 2H), 7.41 (d, J=7.3 Hz, 2H), 7.36 (d, J=8.4 Hz, 2H), 7.17 (dd, J=8.3, 1.9 Hz, 2H), 7.13 (s, 2H), 4.74 (s, 4H), 4.24 (t, J=6.7 Hz, 4H), 3.32 (q, J=6.2 Hz, 5H), 3.23 (d, J=8.0 Hz, 4H), 3.07 (t, J=5.9 Hz, 4H), 1.84 (s, 6H).

LCMS (ESI): calculated for C₄₀H₄₂Cl₂N₄O₆; [M+H]⁺: 773.26, found: 773.55.

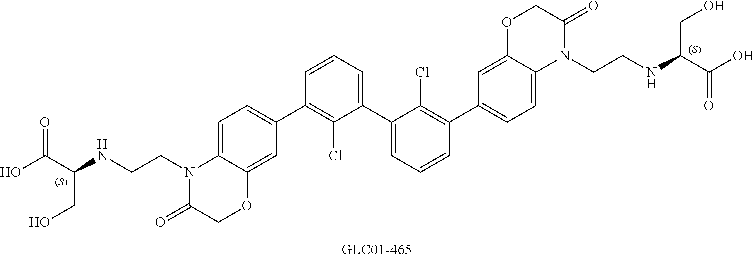

¹H NMR (500 MHz, DMSO-d₆) δ 7.52 (t, J=7.5 Hz, 2H), 7.46 (dd, J=7.7, 1.9 Hz, 2H), 7.41 (dd, J=7.4, 1.8 Hz, 2H), 7.37 (d, J=8.5 Hz, 2H), 7.19 (dd, J=8.4, 2.0 Hz, 2H), 7.13 (d, J=2.0 Hz, 2H), 4.73 (s, 4H), 4.38-4.18 (m, 4H), 4.04 (s, 2H), 3.97-3.77 (m, 4H), 3.22 (t, J=7.1 Hz, 4H), 2.53-2.51 (m, 2H).

LCMS (ESI): calculated for C₃₈H₃₆Cl₂N₄O₁₀; [M+H]⁺: 779.19, found: 779.25.

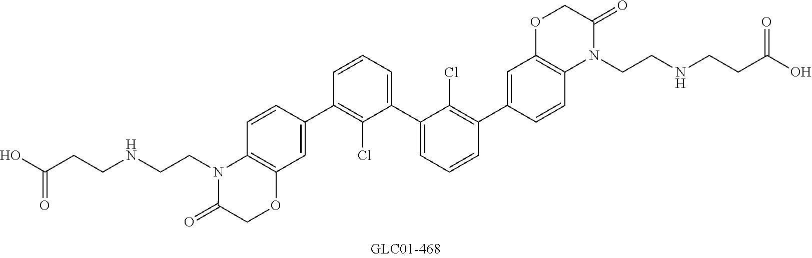 [SMILES: O=C(O)CCNCCN1C(=O)COc2cc(-c3cccc(-c4cccc(-c5ccc6c(c5)OCC(=O)N6CCNCCC(=O)O)c4Cl)c3Cl)ccc21]

¹H NMR (500 MHz, DMSO-d₆) δ 7.52 (t, J=7.5 Hz, 2H), 7.46 (d, J=7.6 Hz, 2H), 7.41 (d, J=7.5 Hz, 2H), 7.35 (d, J=8.4 Hz, 2H), 7.18 (d, J=8.5 Hz, 2H), 7.13 (s, 2H), 4.74 (s, 4H), 4.24 (t, J=5.8 Hz, 4H), 3.24-3.15 (m, 8H), 2.63 (t, J=6.8 Hz, 4H).

LCMS (ESI): calculated for C₃₈H₃₆Cl₂N₄O₈; [M+H]⁺: 747.2, found: 747.2.

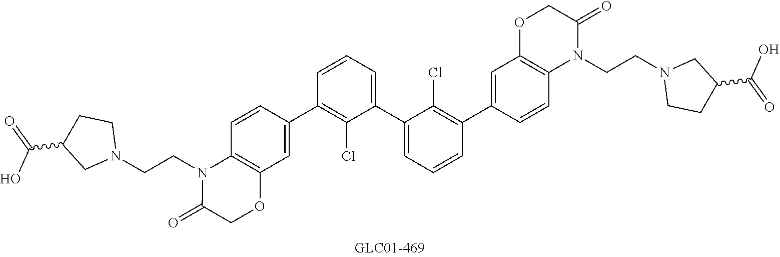 [SMILES: O=C(O)C1CCN(CCN2C(=O)COc3cc(-c4cccc(-c5cccc(-c6ccc7c(c6)OCC(=O)N7CCN6CCC(C(=O)O)C6)c5Cl)c4Cl)ccc32)C1]

¹H NMR (500 MHz, DMSO-d₆) δ 7.52 (t, J=7.5 Hz, 2H), 7.45 (d, J=7.6 Hz, 2H), 7.41 (d, J=7.5 Hz, 2H), 7.37 (d, J=8.4 Hz, 2H), 7.18 (d, J=8.3 Hz, 2H), 7.14 (s, 2H), 4.75 (s, 4H), 4.30 (s, 4H), 3.50-3.30 (m, 14H), 2.20 (d, J=63.2 Hz, 4H)

LCMS (ESI): calculated for C₄₂H₄₀Cl₂N₄O₈; [M+H]⁺: 799.2, found: 799.2.

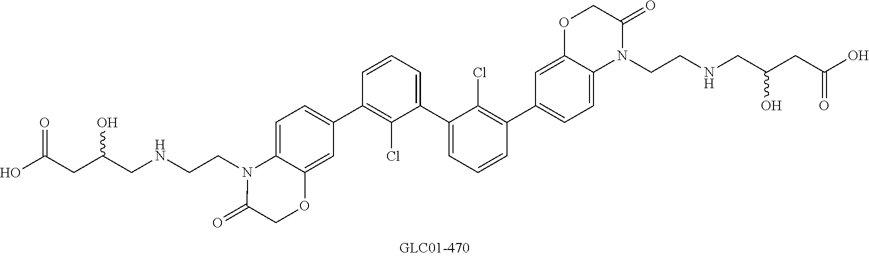 [SMILES: O=C(O)CC(O)CNCCN1C(=O)COc2cc(-c3cccc(-c4cccc(-c5ccc6c(c5)OCC(=O)N6CCNCC(O)CC(=O)O)c4Cl)c3Cl)ccc21]

¹H NMR (500 MHz, DMSO-d₆) δ 7.52 (t, J=7.5 Hz, 2H), 7.46 (d, J=7.4 Hz, 2H), 7.42-7.38 (m, 2H), 7.36 (d, J=8.4 Hz, 2H), 7.18 (d, J=8.2 Hz, 2H), 7.13 (d, J=1.9 Hz, 2H), 4.74 (s, 4H), 4.34-4.18 (m, 4H), 4.15 (s, 2H), 3.20 (d, J=6.9 Hz, 4H), 3.13 (d, J=12.2 Hz, 2H), 2.97 (t, J=11.0 Hz, 2H), 2.45 (d, J=5.4 Hz, 4H), 2.39-2.36 (m, 4H).

LCMS (ESI): calculated for C₄₀H₄₀Cl₂N₄O₁₀; [M+H]⁺: 807.22, found: 807.25.

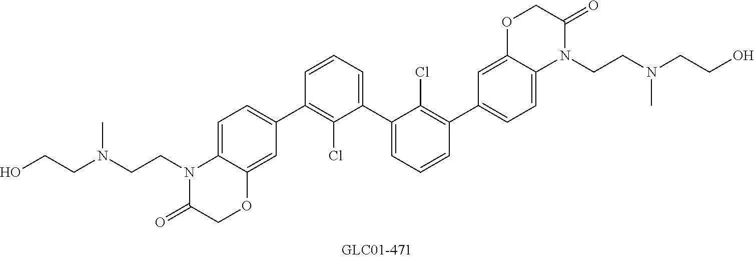 [SMILES: CN(CCO)CCN1C(=O)COc2cc(-c3cccc(-c4cccc(-c5ccc6c(c5)OCC(=O)N6CCN(C)CCO)c4Cl)c3Cl)ccc21]

¹H NMR (500 MHz, DMSO-d₆) δ 9.74 (s, 2H), 7.52 (t, J=7.6 Hz, 2H), 7.48-7.44 (m, 2H), 7.43-7.38 (m, 4H), 7.19 (dd, J=8.4, 2.0 Hz, 2H), 7.14 (d, J=2.0 Hz, 2H), 5.42 (s, 2H), 4.75 (s, 4H), 4.34 (t, J=7.3 Hz, 4H), 3.76 (t, J=5.2 Hz, 4H), 3.50-3.40 (m, 4H), 3.33-3.16 (m, 4H), 2.93 (s, 6H).

LCMS (ESI): calculated for C₃₈H₄₀Cl₂N₄O₆; [M+H]⁺: 719.23, found: 719.52.

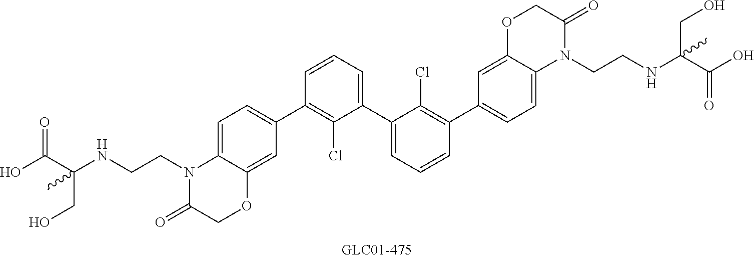

¹H NMR (500 MHz, DMSO-d₆) δ 7.51 (t, J=7.6 Hz, 2H), 7.46 (dd, J=7.6, 1.9 Hz, 2H), 7.40 (dd, J=7.3, 1.9 Hz, 2H), 7.37 (d, J=8.5 Hz, 2H), 7.19 (dd, J=8.3, 2.0 Hz, 2H), 7.13 (d, J=2.1 Hz, 2H), 4.72 (s, 4H), 4.25 (s, 4H), 3.75 (d, J=11.7 Hz, 2H), 3.60 (d, J=11.6 Hz, 2H), 3.11 (d, J=8.6 Hz, 4H), 2.65-2.55 (m, 4H), 1.35 (s, 6H).

LCMS (ESI): calculated for C₄₀H₄₀Cl₂N₄O₁₀; [M+H]⁺: 807.22, found: 807.41.

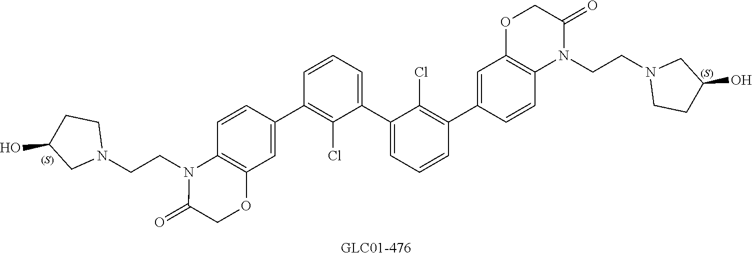

¹H NMR (500 MHz, DMSO-d₆) δ 7.52-7.44 (m, 4H), 7.39 (d, J=7.3 Hz, 2H), 7.29 (d, J=8.3 Hz, 2H), 7.17 (d, J=8.3 Hz, 2H), 7.11 (s, 2H), 4.70 (s, 6H), 4.18 (s, 2H), 4.03 (t, J=7.0 Hz, 4H), 2.80 (dd, J=9.4, 6.2 Hz, 2H), 2.67-2.60 (m, 6H), 2.38 (dd, J=9.9, 3.4 Hz, 2H), 2.03-1.91 (m, 4H), 1.53 (dd, J=8.5, 4.2 Hz, 2H).

LCMS (ESI): calculated for C₄₀H₄₀Cl₂N₄O₆; [M+H]⁺: 743.2, found: 743.2.

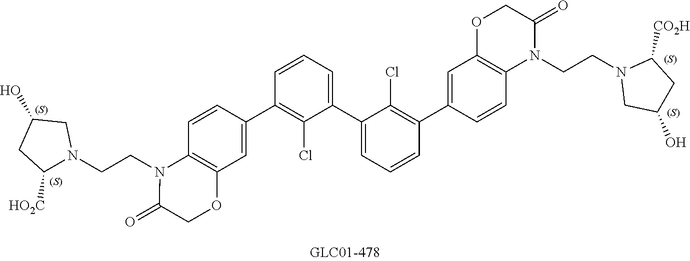

¹H NMR (500 MHz, DMSO-d₆) δ 7.52-7.44 (m, 4H), 7.39 (d, J=7.3 Hz, 2H), 7.29 (d, J=8.3 Hz, 2H), 7.17 (d, J=8.3 Hz, 2H), 7.11 (s, 2H), 4.70 (s, 5H), 4.24-4.18 (m, 3H), 4.03 (t, J=7.0 Hz, 2H) 3.24-3.15 (m, 4H), 2.80 (dd, J=9.4, 6.2 Hz, 1H), 2.67-2.60 (m, 5H). 2.38 (dd, J=9.9, 3.4 Hz, 1H), 2.03-1.91 (m, 2H), 1.53 (dd, J=8.5, 4.2 Hz, 1H).

LCMS (ESI): calculated for C₄₂H₄₀Cl₂N₄O₁₀; [M+H]⁺: 831.2, found: 831.2.

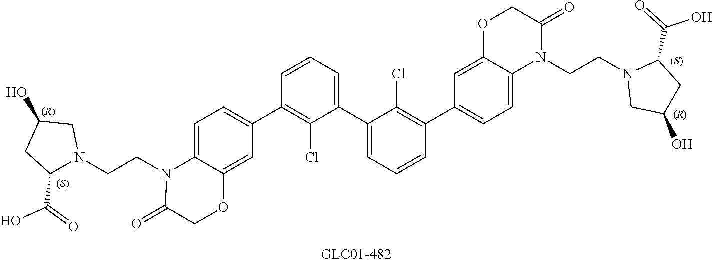

¹H NMR (500 MHz, DMSO-d₆) δ 7.51 (t, J=7.5 Hz, 2H), 7.46 (dd, J=7.8, 1.9 Hz, 2H), 7.40 (dd, J=7.5, 1.9 Hz, 2H), 7.33 (d, J=8.5 Hz, 2H), 7.17 (dd, J=8.2, 2.0 Hz, 2H), 7.12 (d, J=1.9 Hz, 2H), 5.30 (s, 2H), 4.72 (s, 4H), 4.33-4.29 (m, 2H), 4.20-4.12 (m, 4H), 4.07-3.87 (m, 4H), 3.59-3.56 (m, 2H), 3.07-3.02 (m, 2H), 2.88-2.82 (m, 2H), 2.10-2.03 (m, 4H).

LCMS (ESI): calculated for C₄₂H₄₀Cl₂N₄O₁₀; [M+H]⁺: 831.21, found: 831.50.

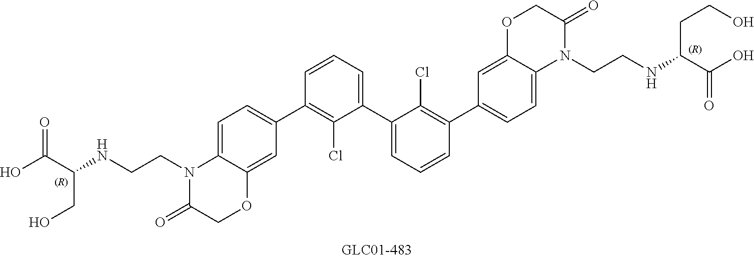

¹H NMR (500 MHz, DMSO-d₆) δ 7.52 (t, J=7.5 Hz, 2H), 7.46 (dd, J=7.7, 1.9 Hz, 2H), 7.41 (dd, J=7.4, 1.8 Hz, 2H), 7.37 (d, J=8.5 Hz, 2H), 7.19 (dd, J=8.4, 2.0 Hz, 2H), 7.13 (d, J=2.0 Hz, 2H), 4.73 (s, 4H), 4.38-4.18 (m, 4H), 4.04 (s, 2H), 3.97-3.77 (m, 4H), 3.22 (t, J=7.1 Hz, 4H), 2.53-2.51 (m, 2H).

LCMS (ESI): calculated for C₃₆H₃₆Cl₂N₄O₆; [M+H]⁺: 779.19, found: 779.25.

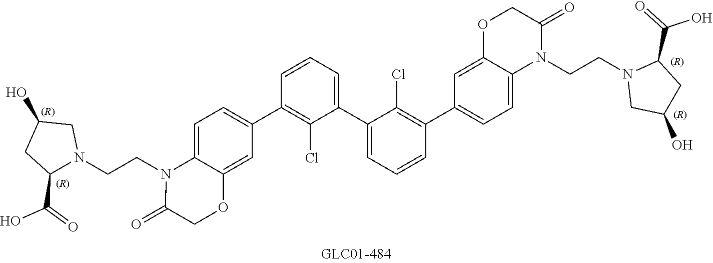

¹H NMR (500 MHz, DMSO-d₆) δ 7.52 (t, J=7.6 Hz, 2H), 7.46 (d, J=7.6 Hz, 2H), 7.41 (d, J=7.7 Hz, 4H), 7.19 (d, J=8.4 Hz, 2H), 7.13 (d, J=2.1 Hz, 2H), 4.73 (s, 4H), 4.39 (s, 2H), 4.35-4.25 (m, 6H), 3.55-3.51 (m, 4H), 3.25-3.20 (m, 4H), 2.65-2.60 (m, 2H), 2.07 (d, J=13.5 Hz, 2H).

LCMS (ESI): calculated for C₄₂H₄₀Cl₂N₄O₁₀; [M+H]⁺: 831.21, found: 831.45.

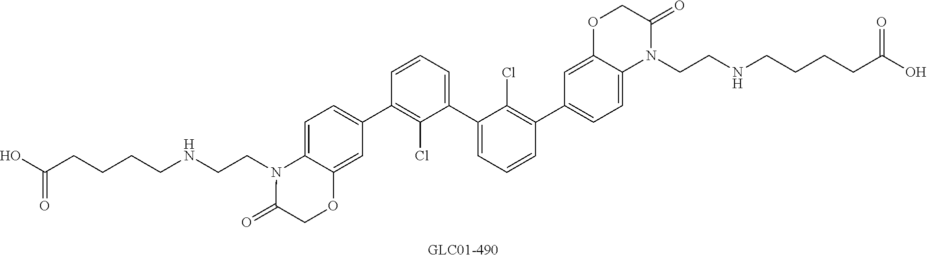 [SMILES: O=C(O)CCCCNCCN1C(=O)COc2cc(-c3cccc(-c4cccc(-c5ccc6c(c5)OCC(=O)N6CCNCCCCC(=O)O)c4Cl)c3Cl)ccc21]

¹H NMR (500 MHz, DMSO-d₆) δ 12.16 (s, 2H), 8.56 (s, 4H), 7.52 (t, J=7.6 Hz, 2H), 7.45 (d, J=7.5 Hz, 2H), 7.41 (d, J=7.4 Hz, 2H), 7.34 (d, J=8.4 Hz, 2H), 7.18 (dd, J=8.2, 2.0 Hz, 2H), 7.14 (d, J=1.9 Hz, 2H), 4.75 (s, 4H), 4.24 (t, J=6.5 Hz, 4H), 3.23-3.16 (m, 4H), 3.03-2.97 (m, 4H), 2.27 (t, J=7.0 Hz, 4H), 1.62-1.52 (m, 8H).

LCMS (ESI): calculated for C₄₂H₄₄Cl₂N₄O₈; [M+H]⁺: 803.25, found: 803.51.

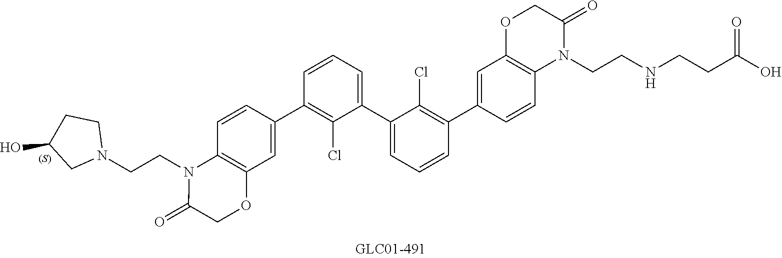

¹H NMR (500 MHz, DMSO-d₆) δ 7.52-7.44 (m, 4H), 7.39 (d, J=7.3 Hz, 2H), 7.29 (d, J=8.3 Hz, 2H), 7.17 (d, J=8.3 Hz, 2H), 7.11 (s, 2H), 4.70 (s, 4H), 4.24-4.18 (m, 3H), 4.03 (t, J=7.0 Hz, 2H) 3.24-3.15 (m, 4H), 2.80 (dd, J=9.4, 6.2 Hz, 1H), 2.67-2.60 (m, 5H). 2.38 (dd, J=9.9, 3.4 Hz, 1H), 2.03-1.91 (m, 2H), 1.53 (dd, J=8.5, 4.2 Hz, 1H).

LCMS (ESI): calculated for C₃₉H₃₈Cl₂N₄O₇; [M+H]⁺: 745.2, found: 745.2.

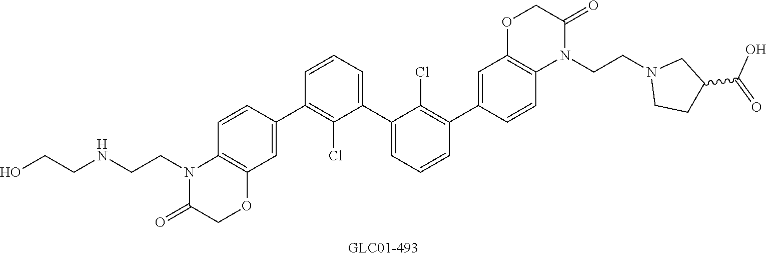 [SMILES: O=C(O)C1CCN(CCN2C(=O)COc3cc(-c4cccc(-c5cccc(-c6ccc7c(c6)OCC(=O)N7CCNCCO)c5Cl)c4Cl)ccc32)C1]

¹H NMR (500 MHz, DMSO-d₆) δ 7.52 (t, J=7.5 Hz, 2H), 7.45 (d, J=7.5 Hz, 2H), 7.41 (d, J=7.5 Hz, 2H), 7.36 (d, J=8.4 Hz, 2H), 7.18 (d, J=8.3 Hz, 2H), 7.14 (s, 2H), 4.75 (s, 4H), 4.29 (s, 4H), 3.65 (s, 1H), 3.40-3.20 (m, 11H), 3.10 (s, 1H), 2.23 (s, 4H) LCMS (ESI): calculated for C₃₉H₃₈Cl₂N₄O₇; [M+H]⁺: 745.2, found: 745.2.

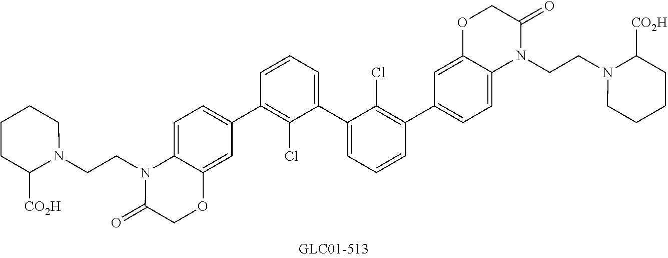 [SMILES: O=C(O)C1CCCCN1CCN1C(=O)COc2cc(-c3cccc(-c4cccc(-c5ccc6c(c5)OCC(=O)N6CCN5CCCCC5C(=O)O)c4Cl)c3Cl)ccc21]

¹H NMR (500 MHz, DMSO-d₆) δ 7.50 (t, J=7.5 Hz, 2H), 7.47-7.44 (m, 2H), 7.41 (t, J=7.9 Hz, 4H), 7.18 (dd, J=8.4, 2.0 Hz, 2H), 7.12-7.09 (m, 2H), 4.67 (s, 4H), 4.08 (t, J=6.4 Hz, 4H), 3.32 (t, J=6.0 Hz, 2H), 3.01-2.93 (m, 4H), 2.92-2.81 (m, 4H), 2.74-2.66 (m, 2H), 1.91-1.81 (m, 4H), 1.67-1.48 (m, 6H).

LCMS (ESI): calculated for C₄₄H₄₄Cl₂N₄O₈; [M+H]⁺: 827.25, found: 827.50.

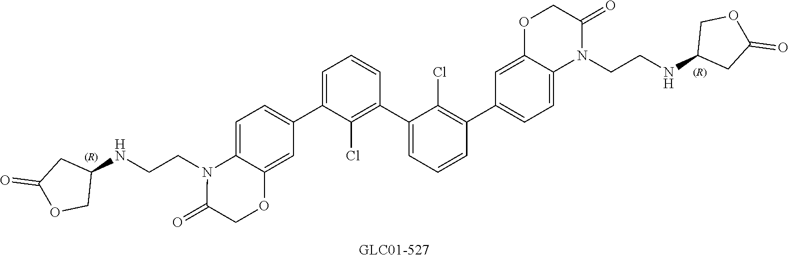

¹H NMR (500 MHz, DMSO-d₆) δ 7.53-7.48 (m, 1H), 7.46 (dd, J=7.6, 1.9 Hz, 1H), 7.40 (dd, J=7.4, 1.9 Hz, 1H), 7.35 (d, J=8.4 Hz, 1H), 7.16 (dd, J=8.3, 2.0 Hz, 1H), 7.10 (d, J=2.1 Hz, 1H), 4.70 (s, 2H), 4.29 (dd, J=9.2, 5.4 Hz, 1H), 4.02 (dd, J=9.3, 2.9 Hz, 1H), 3.98 (t, J=7.2 Hz, 2H), 3.57 (s, 1H), 2.82-2.66 (m, 4H), 2.24 (dd, J=17.2, 3.3 Hz, 1H).

LCMS (ESI): calculated for C₄₀H₃₆Cl₂N₄O₈; [M+H]⁺: 771.20, found: 771.48.

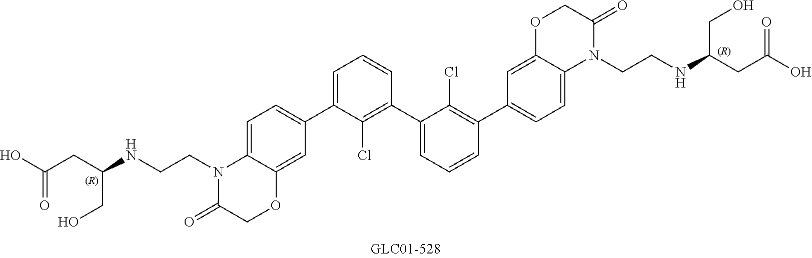

¹H NMR (500 MHz, DMSO-d₆) δ 7.51-7.43 (m, 2H), 7.38 (dd, J=7.8, 1.9 Hz, 1H), 7.31 (d, J=8.3 Hz, 1H), 7.15 (d, J=8.3 Hz, 1H), 7.11 (d, J=2.1 Hz, 1H), 4.82 (s, 1H), 4.70 (s, 2H), 4.01-3.94 (m, 2H), 3.59-3.44 (s, 3H), 2.83-2.64 (m, 4H), 2.25-2.20 (m, 1H).

LCMS (ESI): calculated for C₄₀H₄₀Cl₂N₄O₁₀; [M+H]⁺: 807.22, found: 807.45.

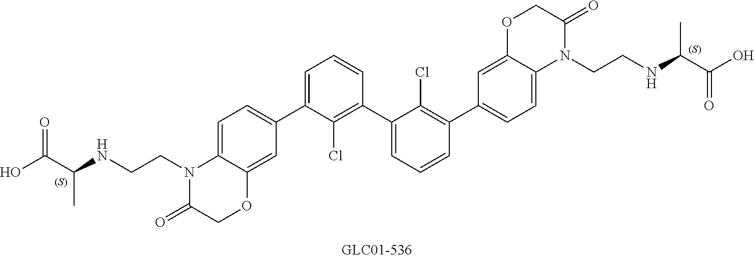

¹H NMR (500 MHz, DMSO-d₆) δ 7.52-7.46 (m, 1H), 7.45 (d, J=7.6 Hz, 1H), 7.40 (dd, J=7.4, 2.1 Hz, 1H), 7.35 (d, J=8.2 Hz, 1H), 7.16 (dd, J=8.2, 2.0 Hz, 1H), 7.10 (d, J=2.1 Hz, 1H), 4.69 (s, 2H), 3.98 (t, J=7.2 Hz, 2H), 3.41-3.33 (m, 1H), 2.78-2.66 (m, 2H), 1.29 (d, J=6.6 Hz, 3H).

LCMS (ESI): calculated for C₃₈H₃₆Cl₂N₄O₈; [M+H]⁺: 747.20, found: 747.41.

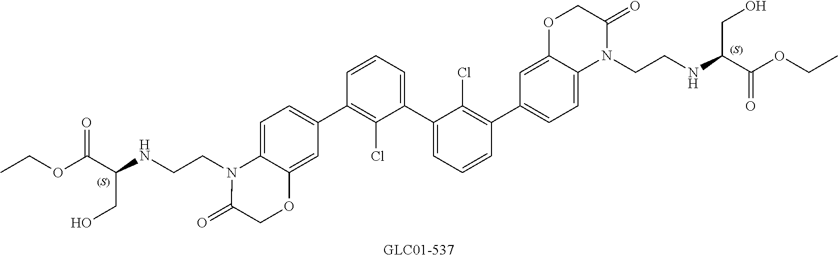

¹H NMR (500 MHz, DMSO-d₆) δ 7.50 (t, J=7.5 Hz, 2H), 7.48-7.43 (m, 2H), 7.39 (dd, J=7.4, 1.7 Hz, 2H), 7.35 (d, J=8.5 Hz, 2H), 7.17-7.13 (m, 2H), 7.11-7.07 (m, 2H), 4.81 (t, J=5.6 Hz, 2H), 4.69 (s, 4H), 4.07 (q, J=7.1 Hz, 4H), 4.02-3.91 (m, 4H), 3.59-3.49 (m 4H), 3.32-3.28 (m, 2H), 2.88-2.78 (m, 2H), 2.72-2.65 (m, 2H), 1.16 (t, J=7.1, 1.0 Hz, 6H).

LCMS (ESI): calculated for C₄₂H₄₄Cl₂N₄O₁₀; [M+H]⁺: 835.24, found: 835.51.

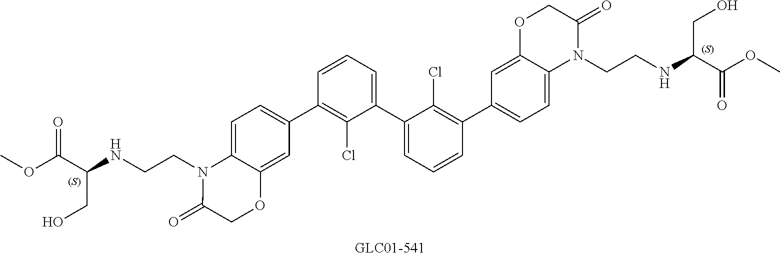

¹H NMR (500 MHz, DMSO-d₆) δ 9.36 (s, 3H), 7.52 (t, J=7.5 Hz, 2H), 7.46 (d, J=6.9 Hz, 2H), 7.41 (d, J=7.5 Hz, 2H), 7.36 (d, J=8.4 Hz, 2H), 7.19 (d, J=8.4 Hz, 2H), 7.13 (d, J=1.9 Hz, 2H), 5.75 (s, 2H), 4.73 (s, 4H), 4.40-4.21 (m, 6H), 3.95-3.87 (m, 4H), 3.78 (s, 6H), 3.30-3.22 (m, 4H).

LCMS (ESI): calculated for C₄₀H₄₀Cl₂N₄O₁₀; [M+H]⁺: 807.21, found: 807.49.

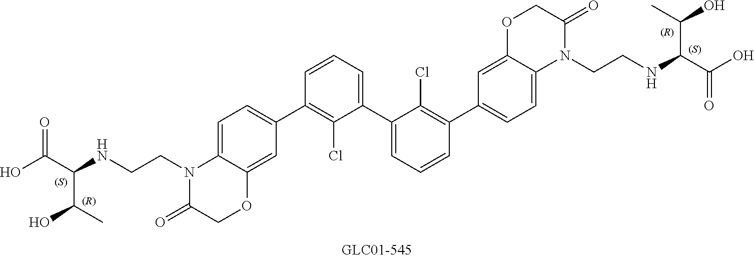

¹H NMR (400 MHz, DMSO-d₆) δ 7.51-7.45 (m, 4H), 7.40-7.37 (m, 4H), 7.15 (d, J=8.0 Hz, 2H), 7.10 (s, 2H), 4.70 (s, 4H), 4.16 (m, 2H), 4.08 (m, 2H), 3.85 (m, 2H), 3.08 (d, J=4 Hz, 2H), 3.04 (m, 2H), 2.87 (m, 2H), 1.13 (d, J=8.0 Hz, 6H).

LCMS (ESI): calculated for C₄₀H₄₀Cl₂N₄O₁₀; [M+H]⁺: 807.21, found: 807.21.

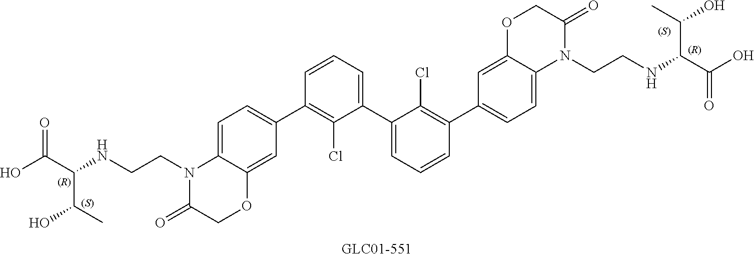

¹H NMR (400 MHz, DMSO-d₆) δ 7.53-7.43 (m, 4H), 7.41-7.36 (m, 4H), 7.20 (d, J=8.0 Hz, 2H), 7.12 (s, 2H), 4.71 (s, 4H), 4.15 (m, 2H), 4.09 (m, 2H), 3.87 (m, 2H), 3.10 (d, J=4 Hz, 2H), 3.05 (m, 2H), 2.90 (m, 2H), 1.14 (d, J=8.0 Hz, 6H).

LCMS (ESI): calculated for C₄₀H₄₀Cl₂N₄O₁₀; [M+H]⁺: 807.21, found: 807.21.

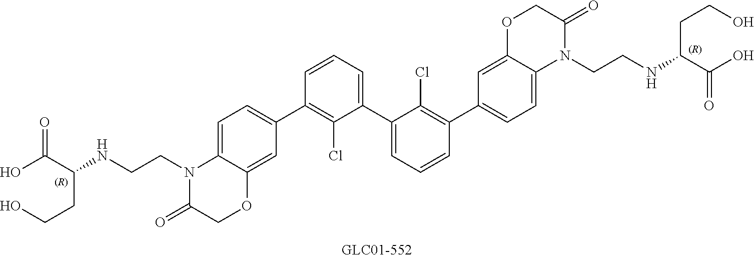

¹H NMR (400 MHz, DMSO-d₆) δ 7.52-7.35 (m, 10H), 7.16 (d, J=8.0 Hz, 2H), 7.11 (s, 2H), 7.02-6.82 (m, 2H), 4.74 (s, 4H), 4.10 (m, 4H), 3.51 (m, 4H), 2.99 (t, J=6 Hz, 4H), 2.87 (m, 2H), 1.78 (m, 2H), 1.67 (m, 2H).

LCMS (ESI): calculated for C₄₀H₄₀Cl₂N₄O₁₀; [M+H]⁺: 807.21, found: 807.21.

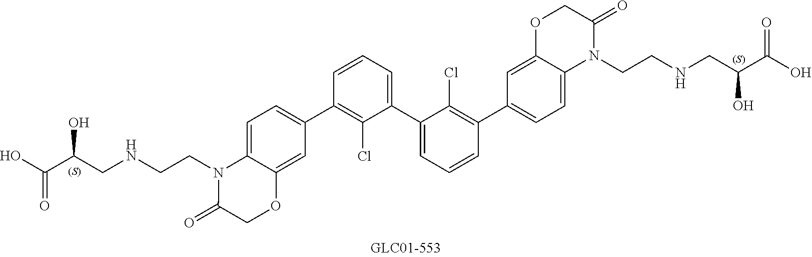

¹H NMR (500 MHz, DMSO-d₆) δ 7.53 (t, J=7.5 Hz, 2H), 7.48 (d, J=6.9 Hz, 2H), 7.43 (d, J=7.5 Hz, 2H), 7.38 (d, J=8.4 Hz, 2H), 7.21 (d, J=8.4 Hz, 2H), 7.15 (d, J=1.9 Hz, 2H), 4.73 (s, 4H), 4.69 (s, 2H), 3.92 (dd, J=6.0, 2.7 Hz, 4H), 3.72-3.65 (m, 4H), 3.31-3.29 (m, 2H), 3.08-2.87 (m, 4H).

LCMS (ESI): calculated for C₃₈H₃₆Cl₂N₄O₁₀; [M+H]⁺: 779.18, found: 779.40.

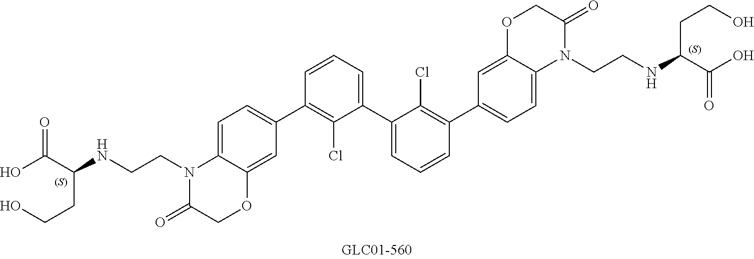

¹H NMR (400 MHz, DMSO-d₆) δ 7.52-7.35 (m, 10H), 7.16 (d, J=8.0 Hz, 2H), 7.11 (s, 2H), 7.02-6.82 (m, 2H), 4.74 (s, 4H), 4.11 (m, 4H), 3.49 (m, 4H), 3.02 (t, J=6 Hz, 4H), 2.90 (m, 2H), 1.81 (m, 2H), 1.69 (m, 2H).

LCMS (ESI): calculated for C₄₀H₄₀Cl₂N₄O₁₀; [M+H]⁺: 807.21, found: 807.21.

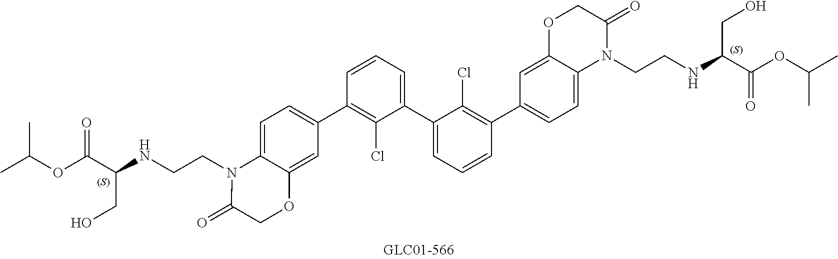

¹H NMR (500 MHz, DMSO-d6) δ 8.20 (s, 1H), 7.50 (t, J=7.5 Hz, 2H), 7.45 (dd, J=7.8, 1.8 Hz, 2H), 7.39 (dd, J=7.5, 1.8 Hz, 2H), 7.35 (d, J=8.4 Hz, 2H), 7.15 (dd, J=8.3, 2.0 Hz, 2H), 7.09 (d, J=2.0 Hz, 2H), 4.92-4.87 (m, 2H), 4.69 (s, 4H), 4.03-3.95 (m, 4H), 3.55-3.51 (m, 6H), 3.29-3.27 (m, 2H), 2.85-2.80 (m, 2H), 2.71-2.64 (m, 2H), 1.16 (t, J=6.6 Hz, 12H).

LCMS (ESI): calculated for C₄₄H₄₈Cl₂N₄O₁₀; [M+H]⁺: 863.27, found: 863.77.

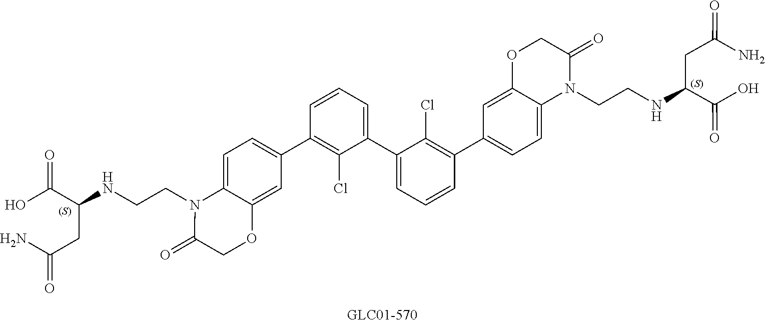

¹H NMR (500 MHz, DMSO-d6) δ 7.7 (s, 2H), 7.52-7.21 (m, 10H), 4.73 (s, 4H), 4.40-4.20 (m, 4H), 3.94-3.90 (m, 2H), 3.50-3.20 (m, 4H), 2.82 (d, J=4.8 Hz, 4H), 2.87 (m, 2H).

LCMS (ESI): calculated for C₄₀H₃₈Cl₂N₆O₁₀; [M+H]⁺: 833.20, found: 833.20.

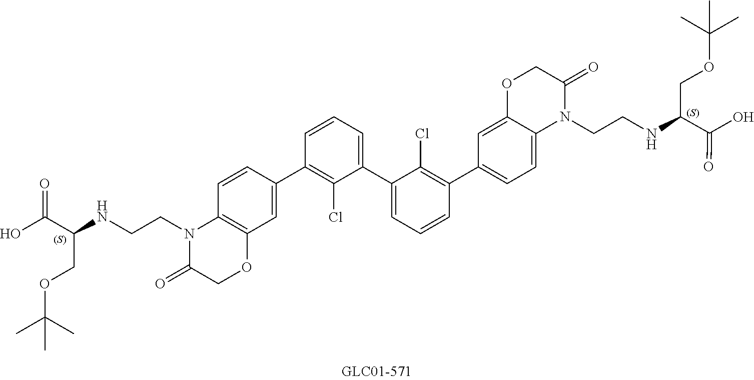

¹H NMR (500 MHz, DMSO-d₆) δ 7.5 (t, J=7.5 Hz, 2H), 7.45 (dd, J=7.7, 1.8 Hz, 4H), 7.42-7.36 (m, 4H), 7.15 (dd, J=8.3, 2.0 Hz, 2H), 4.70 (s, 4H), 4.14 (hept, J=7.2, 6.7 Hz, 4H), 3.61 (dd, J=9.6, 4.1 Hz, 2H), 3.54 (dd, J=9.5, 6.2 Hz, 2H), 3.33 (t, J=5.1 Hz, 2H), 3.33 (p, J=6.0 Hz, 4H), 1.10 (s, 18H).

LCMS (ESI): calculated for C₄₆H₅₂Cl₂N₄O₁₀; [M+H]⁺: 891.31, found: 891.31.

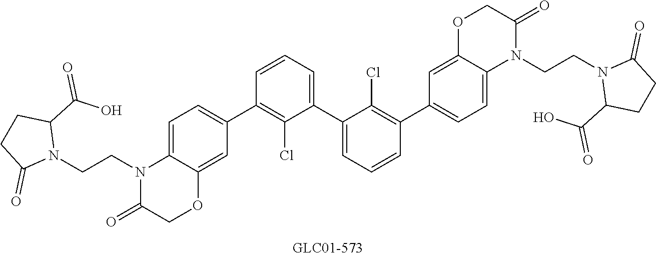 [SMILES: O=C(O)C1CCC(=O)N1CCN1C(=O)COc2cc(-c3cccc(-c4cccc(-c5ccc6c(c5)OCC(=O)N6CCN5C(=O)CCC5C(=O)O)c4Cl)c3Cl)ccc21]

¹H NMR (500 MHz, DMSO-d₆) δ 13.07 (s, 2H), 7.52-7.38 (m, 10H), 7.10 (s, 2H), 4.87 (s, 4H), 4.4-4.3 (m, 2H), 4.22-4.15 (m, 4H), 4.01-3.95 (m, 4H), 3.87-3.79 (m, 4H), 3.11-3.04 (m, 4H).

LCMS (ESI): calculated for C₄₂H₃₆Cl₂N₄O₁₀; [M+H]⁺: 827.18, found: 827.18.

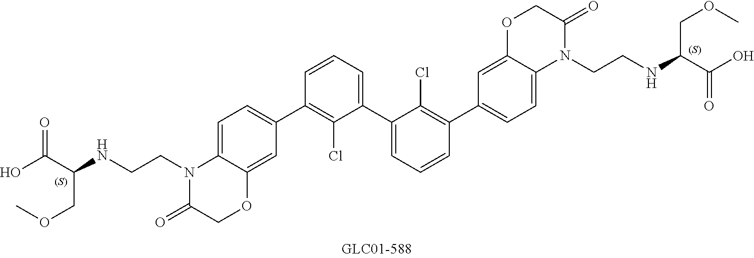

¹H NMR (500 MHz, DMSO-d₆) δ 7.51 (t, J=7.5 Hz, 2H), 7.46 (dd, J=7.7, 1.9 Hz, 2H), 7.42-7.35 (m, 4H), 7.17 (dd, J=8.3, 1.9 Hz, 2H), 7.11 (d, J=2.0 Hz, 2H), 4.71 (s, 4H), 4.18 (q, J=11.6, 9.3 Hz, 4H), 3.66 (m, 4H), 3.26 (s, 6H), 3.07 (m, 4H).

LCMS (ESI): calculated for C₄₀H₄₀Cl₂N₄O₁₀; [M+H]⁺: 807.21, found: 807.21.

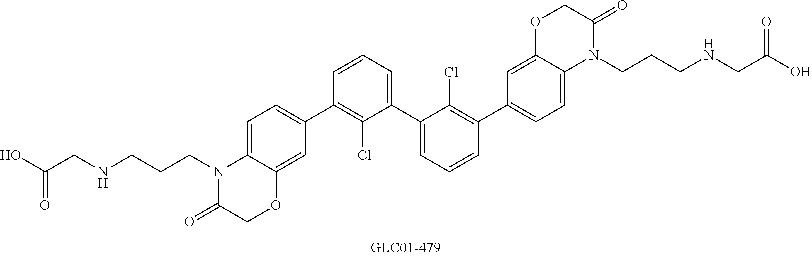 [SMILES: O=C(O)CNCCCN1C(=O)COc2cc(-c3cccc(-c4cccc(-c5ccc6c(c5)OCC(=O)N6CCCNCC(=O)O)c4Cl)c3Cl)ccc21]

¹H NMR (500 MHz, DMSO-d₆) δ 7.52 (t, J=7.6 Hz, 2H), 7.46 (dd, J=7.8, 1.8 Hz, 2H), 7.40 (dd, J=7.4, 1.9 Hz, 2H), 7.35 (d, J=8.4 Hz, 2H), 7.18 (dd, J=8.1, 2.0 Hz, 2H), 7.13 (d, J=2.0 Hz, 2H), 4.73 (s, 4H), 4.01 (t, J=7.0 Hz, 4H), 3.81 (s, 4H), 3.04-3.00 (m, 4H), 1.97 (t, J=8.1 Hz, 4H).

LCMS (ESI): calculated for C₃₈H₃₆Cl₂N₄O₈; [M+H]⁺: 747.19, found: 747.46.

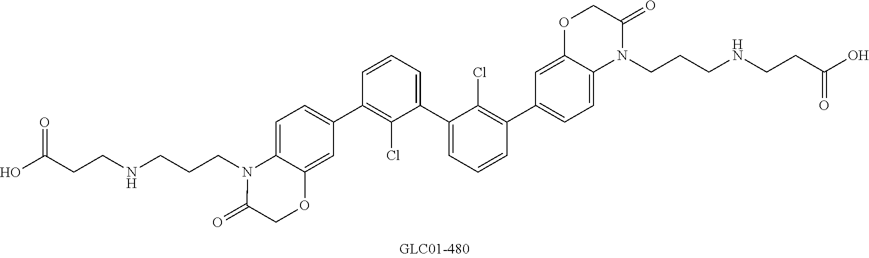 [SMILES: O=C(O)CCNCCCN1C(=O)COc2cc(-c3cccc(-c4cccc(-c5ccc6c(c5)OCC(=O)N6CCCNCCC(=O)O)c4Cl)c3Cl)ccc21]

¹H NMR (500 MHz, DMSO-d₆) δ 7.51 (t, J=7.6 Hz, 2H), 7.45 (dd, J=7.7, 1.8 Hz, 2H), 7.40 (dd, J=7.4, 1.8 Hz, 2H), 7.35 (d, J=8.4 Hz, 2H), 7.18 (dd, J=8.3, 2.0 Hz, 2H), 7.13 (d, J=1.9 Hz, 2H), 4.74 (s, 4H), 4.01 (t, J=7.1 Hz, 4H), 3.12 (t, J=7.0 Hz, 4H), 3.03 (t, J=7.8 Hz, 4H), 2.61 (d, J=7.0 Hz, 4H), 1.98-1.90 (m, 4H).

LCMS (ESI): calculated for C₄₀H₄₀Cl₂N₄O₈; [M+H]⁺: 775.22, found: 775.50.

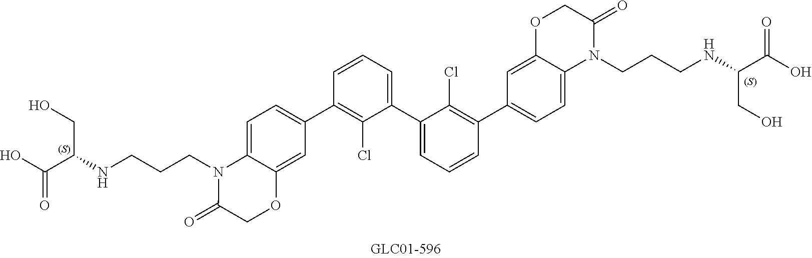

¹H NMR (500 MHz, DMSO-d₆) δ 7.52 (t, J=7.6 Hz, 2H), 7.46 (dd, J=7.8, 1.8 Hz, 2H), 7.40 (dd, J=7.4, 1.9 Hz, 2H), 7.35 (d, J=8.4 Hz, 2H), 7.18 (dd, J=8.1, 2.0 Hz, 2H), 7.13 (d, J=2.0 Hz, 2H), 4.73 (s, 4H), 4.13 (t, J=7.5 Hz, 4H), 4.01 (t, J=7.0 Hz, 4H), 3.31-3.29 (m, 2H), 3.04-3.00 (m, 4H), 1.97 (t, J=8.1 Hz, 4H).

LCMS (ESI): calculated for C₄₀H₄₀Cl₂N₄O₁₀; [M+H]⁺: 807.21, found: 807.50.

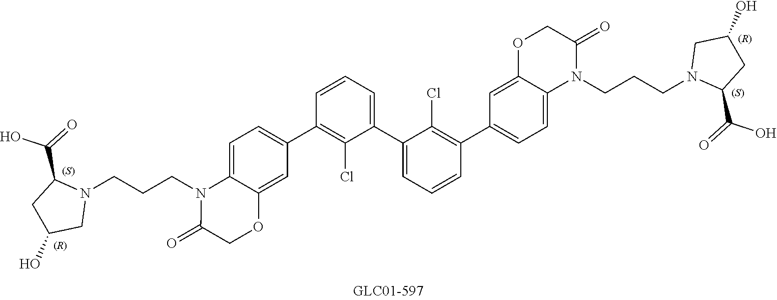

¹H NMR (500 MHz, DMSO-d₆) δ 7.52 (t, J=7.6 Hz, 2H), 7.46 (dd, J=7.8, 1.8 Hz, 2H), 7.40 (dd, J=7.4, 1.9 Hz, 2H), 7.35 (d, J=8.4 Hz, 2H), 7.18 (dd, J=8.1, 2.0 Hz, 2H), 7.13 (d, J=2.0 Hz, 2H), 5.47 (s, 2H), 4.73 (s, 4H), 4.39-4.34 (m, 2H), 4.28-4.19 (m, 4H), 4.01 (t, J=7.0 Hz, 4H), 3.71-3.67 (m, 2H), 3.24-3.20 (m, 2H), 2.68-2.56 (m, 2H), 2.46-2.34 (m, 2H), 2.20-2.11 (m, 4H)

LCMS (ESI): calculated for C₄₄H₄₄Cl₂N₄O₁₀; [M+H]⁺: 859.24, found: 859.50.

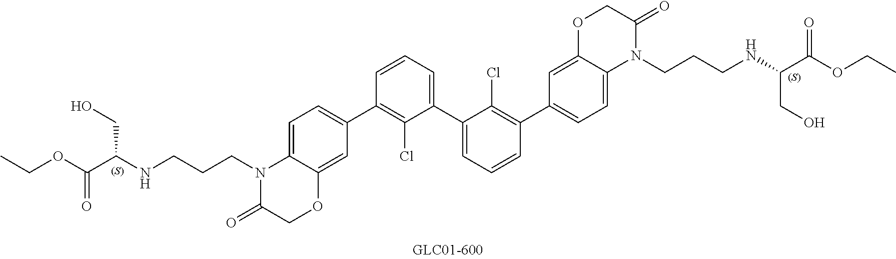

¹H NMR (500 MHz, DMSO-d₆) δ 7.52 (t, J=7.6 Hz, 2H), 7.46 (dd, J=7.8, 1.8 Hz, 2H), 7.40 (dd, J=7.4, 1.9 Hz, 2H), 7.35 (d, J=8.4 Hz, 2H), 7.18 (dd, J=8.1, 2.0 Hz, 2H), 7.13 (d, J=2.0 Hz, 2H), 4.73 (s, 4H), 4.13 (t, J=7.5 Hz, 4H), 4.07 (q, J=7.1 Hz, 4H), 4.01 (t, J=7.0 Hz, 4H), 3.31-3.29 (m, 2H), 3.04-3.00 (m, 4H), 1.97 (t, J=8.1 Hz, 4H), 1.19-1.14 (t, J=7.1 Hz, 6H).

LCMS (ESI): calculated for C₄₄H₄₈Cl₂N₄O₁₀; [M+H]⁺: 863.27, found: 863.50.

### Example 6

### Preparation of (2S,2'S)-2,2′-(((2R,2′R)-((2,2′-dichloro-[1,1′-biphenyl]-3,3′-diyl)bis(3-oxo-2,3-dihydro-4H-benzo[b][1,4]oxazine-7,4-diyl))bis(propane-1,2-diyl))bis(azanediyl))bis(3-hydroxypropanoic acid)

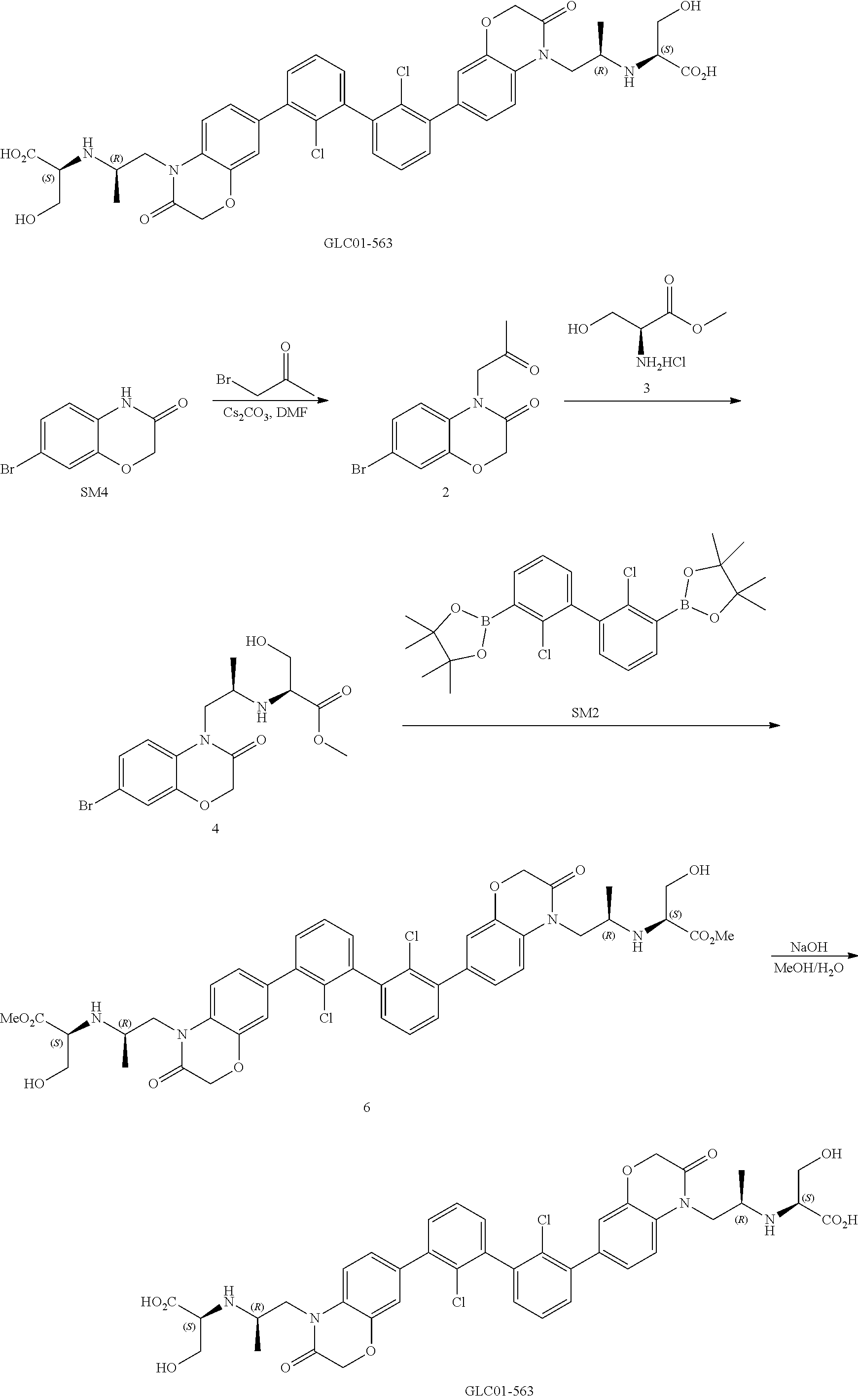

### Synthesis of Intermediate 2

A solution of compound SM4 (0.515 g, 2.258 mmol, 1 eq), 1-bromopropan-2-one (0.340 g, 2.48 mmol, 1.10 eq), Cs₂CO₃ (0.960 g, 2.93 mmol, 1.30 eq) in DMF (5 ml) was stirred at rt for 1 h, 15 mL H₂O was slowly added, the solid was collected and dried to give the title compound 2 (0.400 g, 62%).

### Synthesis of Intermediate 4

To a solution of compound SM2 (0.300 g, 1.056 mmol, 1 eq), compound 3 (0.315 g, 2.024 mmol, 1.90 eq), DIEA (0.274 g, 2.124 mmol, 2.01 eq), 4A and 2 drops of AcOH in DCM (6 ml) was added NaBH(OAc)₃ (1.00 g, 4.73 mmol, 4 eq) at rt. After 3-4 h, 10 mL H₂O was added, extracted by DCM, the organic layer was collected and purified by silica gel to give the title compound 4 (0.07 g, 17%).

### Synthesis of Intermediate 6

A solution of compound SM2 (0.043 g, 0.09 mmol, 1 eq), compound 4 (0.07 g, 0.18 mmol, 2.0 eq), K₂CO₃ (0.05 g, 0.36 mmol, 4.0 eq), Pd(dppf)Cl₂·DCM (0.008 g, 0.009 mmol, 0.1 eq) in dioxane (3 ml) and H₂O (0.5 ml) was stirred at 80° C. for 2 h under N₂, 5 mL H₂O was added, extracted by EA, the organic layer was collected and purified by Pre-TLC to give the title compound 6 (0.05 g, 66%).

### Synthesis of GLC01-563

To a stirred solution of compound 6 (0.025 g, 0.030 mmol, 1 eq) in MeOH (2 mL) was added a solution of NaOH (0.020 g, 0.50 mmol, 16.6 eq) in H₂O (0.5 mL) at room temperature. After 1 h, the mixture was purified by reverse phase HPLC (0.1% trifluoroacetic acid in water/acetonitrile) providing GLC01-563 (12 mg, 50%).

¹H NMR (500 MHz, DMSO-d₆) δ 7.49 (d, J=7.4 Hz, 2H), 7.46 (d, J=7.4 Hz, 2H), 7.40 (t, J=8.0 Hz, 4H), 7.14 (d, J=8.5 Hz, 2H), 7.11 (d, 2H), 4.71 (s, 4H), 4.12-4.07 (m, 2H), 4.00-3.96 (m, 2H), 3.63-3.57 (m, 6H), 3.24-3.22 (m, 2H), 1.08 (d, J=6.4 Hz, 6H).

LCMS (ESI): calculated for C₄₀H₄₀Cl₂N₄O₁₀; [M+H]⁺: 807.21, found: 807.43.

GLC01-550 was prepared using the same procedure

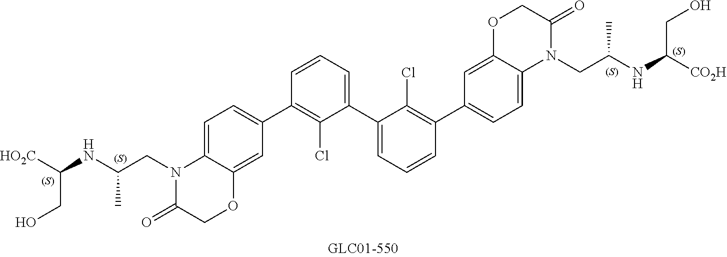

¹H NMR (500 MHz, DMSO-d₆) δ 7.49 (d, J=7.4 Hz, 2H), 7.46 (d, J=7.4 Hz, 2H), 7.40 (t, J=8.0 Hz, 4H), 7.14 (d, J=8.5 Hz, 2H), 7.11 (d, 2H), 4.71 (s, 4H), 4.12-4.07 (m, 2H), 4.00-3.96 (m, 2H), 3.63-3.57 (m, 6H), 3.24-3.22 (m, 2H), 1.08 (d, J=6.4 Hz, 6H).

LCMS (ESI): calculated for C₄₀H₄₀Cl₂N₄O₁₀; [M+H]⁺: 807.21, found: 807.43.

### Example 7

### 7A. Preparation of diethyl 2,2′-((((2,2′-dichloro-[1,1′-biphenyl]-3,3′-diyl)bis(6-fluoro-3-oxo-2,3-dihydro-4H-benzo[b][1,4]oxazine-7,4-diyl))bis(ethane-2,1-diyl))bis(azanediyl))(2S,2'S)-bis(3-hydroxypropanoate) and (2S,2'S)-2,2′-((((2,2′-dichloro-[1,1′-biphenyl]-3,3′-diyl)bis(6-fluoro-3-oxo-2,3-dihydro-4H-benzo[b][1,4]oxazine-7,4-diyl))bis(ethane-2,1-diyl))bis(azanediyl))bis(3-hydroxypropanoic acid)

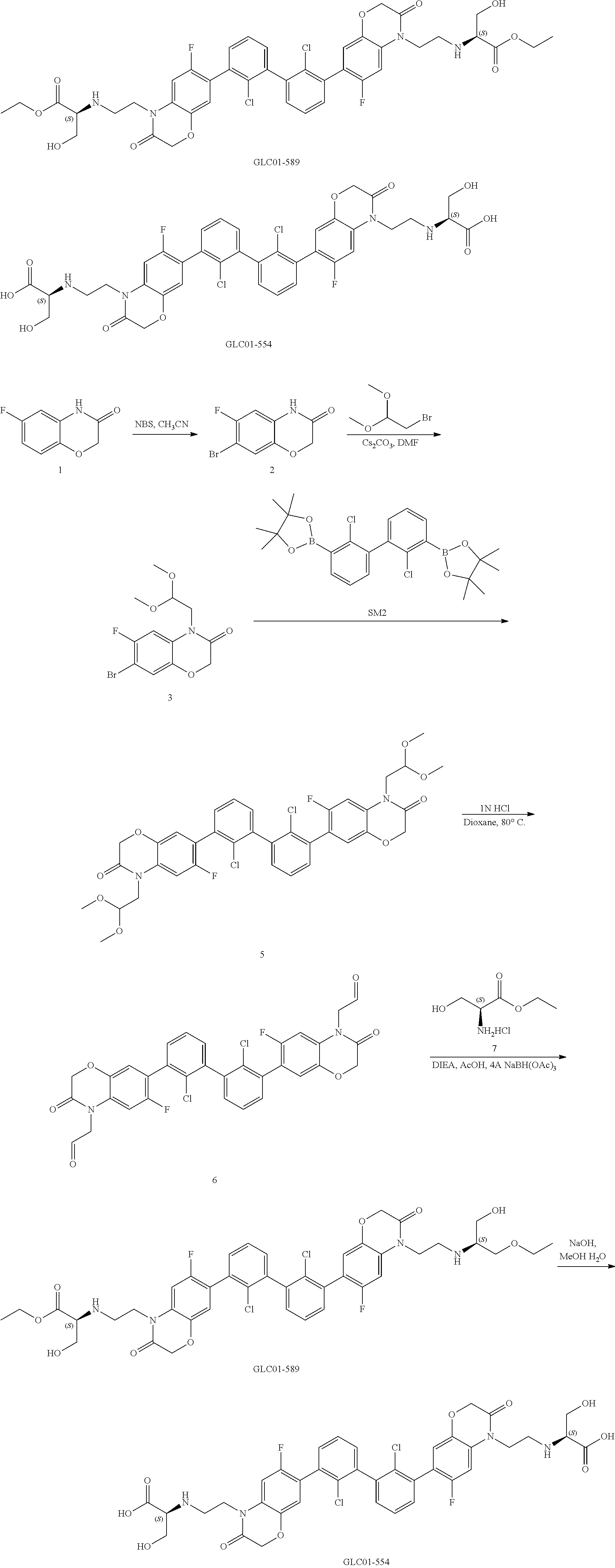

### Synthesis of Intermediate 2

A solution of compound 1 (0.100 g, 0.598 mmol, 1.00 eq), NBS (0.115 g, 0.646 mmol, 1.08 eq), a drop AcOH in CH₃CN (1.5 ml) was stirred at 50° C. for 16 h, H₂O (5 ml) was slowly added, extracted with EA, the organic was concentrated to give the title compound 2 (0.100 g, 68%).

### Synthesis of Intermediate 3

A solution of compound 2 (1.50 g, 6.09 mmol, 1.00 eq), 2-bromo-1,1-dimethoxyethane (2.00 g, 11.83 mmol, 1.94 eq), Cs₂CO₃ (5.00 g, 15.33 mmol, 2.51 eq) in DMF (15 ml) was stirred at 60° C. for 16 h, 45 mL H₂O was slowly added, collected the solid dried in vacuo to give the compound 3 (0.65 g, 32%).

### Synthesis of Intermediate 5

A solution of compound SM2 (0.280 g, 0.589 mmol, 1.00 eq), compound 3 (0.400 g, 1.197 mmol, 2.03 eq), K₂CO₃ (0.350 g, 2.536 mmol, 4.3 eq), Pd(dppf)Cl₂·DCM (0.030 g, 0.036 mmol, 0.06 eq) in dioxane (3 ml) and H₂O (0.5 ml) was stirred at 80° C. for 2 h under N₂, 5 mL H₂O was added, extracted by EA, the organic layer was concentrated and purified by silica gel (PE:EA=3:1-1:1) to give the compound 5 (0.330 g, 77%).

### Synthesis of Intermediate 6

A solution of compound 5 (0.340 g, 0.466 mmol, 1 eq) in dioxane/1N HCl (4 ml/2 ml) was stirred at 80° C. for 0.5 hour, 10 mL H₂O was added, extracted by EA, the organic layer was concentrated to give the compound 6 (0.330 g, crude).

### Synthesis of GLC01-589

To a solution of compound 6 (0.330 g, 0.520 mmol, 1.0 eq), compound 7 (0.440 g, 2.59 mmol, 5.0 eq), DIEA (0.351 g, 2.72 mmol, 5.2 eq), 4A and AcOH (0.155 g, 2.59 mmol, 5.0 eq) in DCM (7 ml) was added NaBH(OAc)₃ (0.500 g, 2.37 mmol, 4.5 eq) at rt. After 3-4 h, 10 mL H₂O was added, extracted by DCM, the organic layer was collected and purified by silica gel (DCM:MeOH=50:1-20:1) to give the GLC01-589 (0.200 g, 44%).

¹H NMR (500 MHz, DMSO-d₆) δ 7.53 (t, J=7.6 Hz, 2H), 7.49-7.43 (m, 4H), 7.37 (d, J=11.2 Hz, 2H), 7.04 (d, J=6.8 Hz, 2H), 4.81 (t, J=5.6 Hz, 2H), 4.68 (s, 4H), 4.07 (q, J=7.1 Hz, 4H), 4.00-3.90 (m, 4H), 3.60-3.49 (m, 4H), 3.37-3.33 (m, 2H), 2.87-2.77 (m, 2H), 2.68-2.62 (m, 2H), 1.19-1.14 (t, J=7.1 Hz, 6H).

LCMS (ESI): calculated for C₄₂H₄₂Cl₂F₂N₄O₁₀; [M+H]⁺: 871.22, found: 871.50.

### Synthesis of GLC01-554

To a solution of GLC01-589 (0.015 g, 0.017 mmol, 1 eq) of MeOH (0.2 ml) was added a solution of NaOH (0.020 g, 0.5 mmol, 29 eq) in H₂O (0.1 ml) at rt. After 1 h, the mixture was purified by reverse phase HPLC (0.10% trifluoroacetic acid in water/acetonitrile) providing GLC01-554 (7.7 mg, 55%).

¹H NMR (500 MHz, DMSO-d₆) δ 7.53 (t, J=7.6 Hz, 2H), 7.48-7.39 (m, 6H), 7.06 (d, J=6.9 Hz, 2H), 4.70 (s, 4H), 4.13 (t, J=7.5 Hz, 4H), 3.72-3.65 (m, 4H), 3.31-3.29 (m, 2H), 3.07-3.03 (m, 2H), 3.02-2.97 (m, 2H).

LCMS (ESI): calculated for C₃₈H₃₄Cl₂F₂N₄O₁₀; [M+H]⁺: 815.16, found: 815.33.

### 7B. The Following Compound could be Prepared Using Different Starting Material

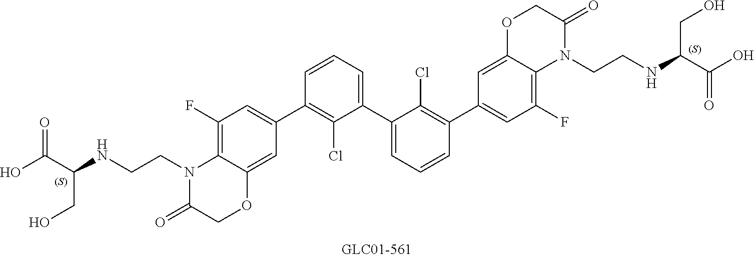

¹H NMR (400 MHz, DMSO-d₆) δ 7.54 (m, 2H), 7.49 (d, J=4 Hz, 2H), 7.43 (d, J=4.0 Hz, 2H), 7.20 (d, J=8 Hz, 2H), 7.06 (s, 2H), 4.72 (s, 4H), 4.26 (m, 4H), 4.14 (s, 2H), 3.90 (m, 4H), 3.34 (m, 4H).

LCMS (ESI): calculated for C₃₈H₃₄F₂Cl₂N₄O₁₀; [M+H]⁺: 815.16, found: 815.16.

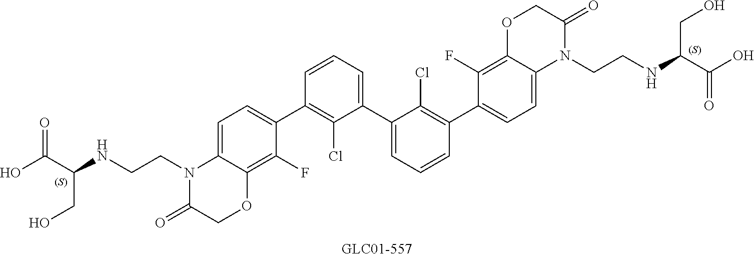

¹H NMR (400 MHz, DMSO-d₆) δ 7.54 (m, 2H), 7.49 (d, J=4 Hz, 2H), 7.43 (d, J=4.0 Hz, 2H), 7.30 (d, J=8 Hz, 2H), 7.06 (s, 2H), 4.72 (s, 4H), 4.26 (m, 4H), 4.14 (s, 2H), 3.90 (m, 4H), 3.34 (m, 4H).

LCMS (ESI): calculated for C₃₈H₃₄F₂Cl₂N₄O₁₀; [M+H]⁺: 815.16, found: 815.16.

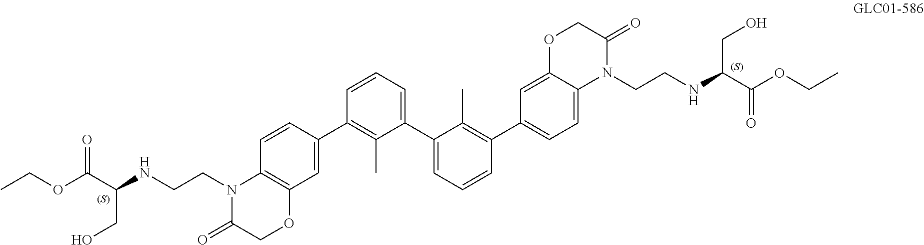

¹H NMR (500 MHz, DMSO-d₆) δ 7.31 (dd, J=8.1, 5.3 Hz, 4H), 7.21 (d, J=7.7 Hz, 2H), 7.14 (d, J=7.5 Hz, 2H), 7.06 (dd, J=8.3, 1.7 Hz, 2H), 7.01 (d, J=2.0 Hz, 2H), 4.82 (s, 2H), 4.68 (s, 4H), 4.07 (q, J=7.1 Hz, 4H), 4.03-3.90 (m, 4H), 3.56 (hept, J=5.1 Hz, 4H), 2.85 (dd, J=12.3 6.8 Hz, 2H), 2.69 (dq, J=13.3, 7.1, 6.3 Hz, 2H), 1.94 (s, 6H), 1.16 (t, J=7.0 Hz, 6H),

LCMS (ESI): calculated for C₄₄H₅₀N₄O₁₀; [M+H]⁺: 795.35, found: 795.35.

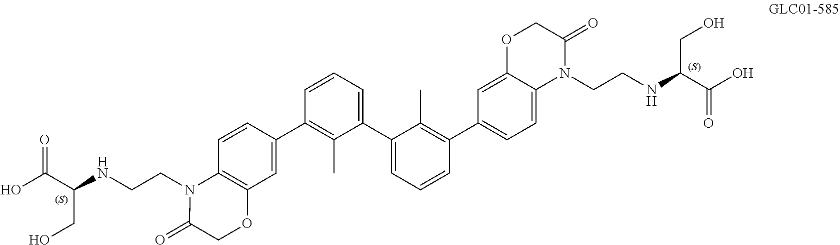

¹H NMR (500 MHz, DMSO-d₆) δ 9.16 (s, 3H), 7.42-7.28 (m, 4H), 7.22 (d, J=7.7 Hz, 2H), 7.15 (d, J=7.5 Hz, 2H), 7.10 (dd, J=8.2, 1.9 Hz, 2H), 7.05 (d, J=2.0 Hz, 2H), 5.71 (s, 1H), 4.72 (s, 4H), 4.29 (hept, J=7.5, 7.0 Hz, 4H), 4.18 (d, J=3.4 Hz, 2H), 3.91 (d, J=3.4 Hz, 4H), 3.25 (t, J=7.7 Hz, 4H), 1.95 (s, 6H), LCMS (ESI): calculated for C₄₀H₄₂N₄O₁₀; [M+H]⁺: 739.29, found: 739.50.

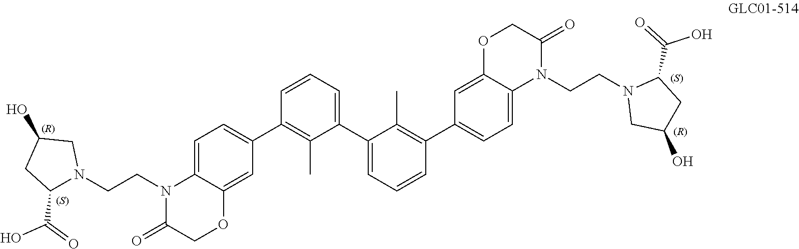

¹H NMR (500 MHz, DMSO-d₆) δ 7.33 (dd, J=8.3, 5.4 Hz, 4H), 7.22 (d, J=7.6 Hz, 2H), 7.15 (d, J=7.5 Hz, 2H), 7.08 (dd, J=8.2, 1.8 Hz, 2H), 7.05 (d, J=2.0 Hz, 2H), 5.47 (s, 2H), 4.72 (s, 4H), 4.39-4.34 (m, 2H), 4.28-4.19 (m, 4H), 3.71-3.67 (m, 2H), 3.24-3.20 (m, 2H), 3.08-3.02 (m, 2H), 2.68-2.56 (m, 2H), 2.46-2.34 (m, 2H), 2.20-2.11 (m, 4H), 1.95 (s, 6H).

LCMS (ESI): calculated for C₄₄H₄₆N₄O₁₀; [M+H]⁺: 791.32, found: 791.58.

### Example 8

### 8A. Preparation of diethyl 2,2′-((((2,2′-difluoro-[1,1′-biphenyl]-3,3′-diyl)bis(3-oxo-2,3-dihydro-4H-benzo[b][1,4]oxazine-7,4-diyl))bis(ethane-2,1-diyl))bis(azanediyl))(2S,2'S)-bis(3-hydroxypropanoate) and (2S,2'S)-2,2′-((((2,2′-difluoro-[1,1′-biphenyl]-3,3′-diyl)bis(3-oxo-2,3-dihydro-4H-benzo[b][1,4]oxazine-7,4-diyl))bis(ethane-2,1-diyl))bis(azanediyl))bis(3-hydroxypropanoic acid)

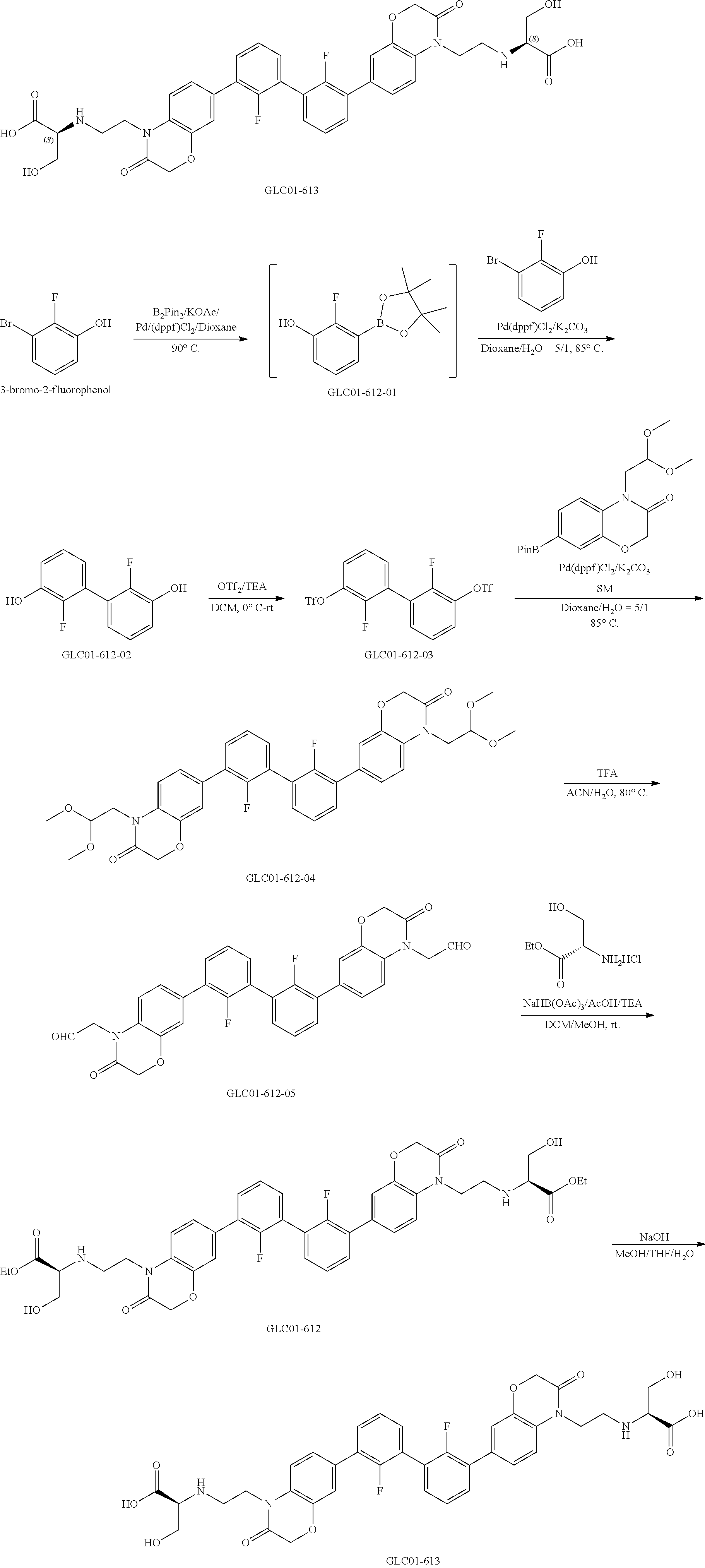

### Synthesis of Intermediate GLC01-612-04

To a stirred solution of GLC01-612-03 (100 mg, 0.206 mmol, 1.0 eq.) and SM (164 mg, 0.453 mmol, 2.2 eq) in 4 ml 1,4-dioxane and 0.8 ml water were added K₂CO₃ (114 mg, 0.826 mmol, 4.0 equiv) and Pd(dppf)Cl₂·DCM (33 mg, 0.04 mmol, 0.2 eq) at room temperature under nitrogen atmosphere. Then the resulting mixture was heated at 85 degrees C. for 2 h. The water (15 ml) was added to dilute the reaction mixture, extracted with EA (3×15 ml). The combined organic phase, washed with 15 ml saturated brine, dried over anhydrous sodium sulfate, filtered, the filtrate was concentrated under vacuum. The residue was purified by prep-TLC (PE/EA=2/1) to afford desired product (70 mg, 52% yield) as a light yellow solid.

### Synthesis of GLC01-612

To a stirred mixture of GLC01-612-04 (50 mg) in ACN (3 ml) and water (3 ml) was added TFA (0.3 ml) at ambient temperature. After stirring for 1 h at 80° C., The resulting solution was adjust pH=8-9 with K₂CO₃ aq., extracted with EA, washed with brine, the organic phase was concentrated under reduce pressure, the residue (43 mg, 100% yield) was used directly in the next step. The reductive amination step was conducted following the above procedure.

¹H NMR (500 MHz, DMSO) δ 7.53-7.31 (m, 8H), 7.12 (dd, J=31.2, 8.5 Hz, 4H), 4.81 (t, J=5.5 Hz, 2H), 4.68 (s, 4H), 4.10-4.03 (m, 4H), 3.98 (ddd, J=21.0, 14.1, 7.1 Hz, 4H), 3.54 (ddt, J=16.0, 10.5, 5.2 Hz, 4H), 2.83 (dt, J=14.1, 7.1 Hz, 2H), 2.69-2.63 (m, 2H), 1.22-1.12 (m, 6H).

LCMS (ESI): calculated for C₄₂H₄₄F₂N₄O₁₀; [M+H]⁺: 803.3, found: 803.3.

### Synthesis of GLC01-613

Hydrolysis reaction was conducted following the above procedure.

¹H NMR (500 MHz, DMSO) δ 7.52 (t, J=7.5 Hz, 2H), 7.46 (dd, J=7.6, 1.8 Hz, 2H), 7.43-7.34 (m, 4H), 7.19 (dd, J=8.4, 1.9 Hz, 2H), 7.13 (d, J=1.9 Hz, 2H), 4.73 (s, 4H), 4.31-4.20 (m, 4H), 4.10-4.01 (m, 2H), 3.90-3.81 (m, 4H), 3.25-3.19 (m, 4H).

LCMS (ESI): calculated for C₃₈H₃₆F₂N₄O₁₀; [M+H]⁺: 747.3, found: 747.3.

### Example 9 (Preparation of Different Core, Same Side)

### 9A. Preparation of 6,6′-(2,2′-dichloro-[1,1′-biphenyl]-3,3′-diyl)bis(1-(((S)-5-oxopyrrolidin-2-yl)methyl)-3,4-dihydroquinolin-2(1H)-one)

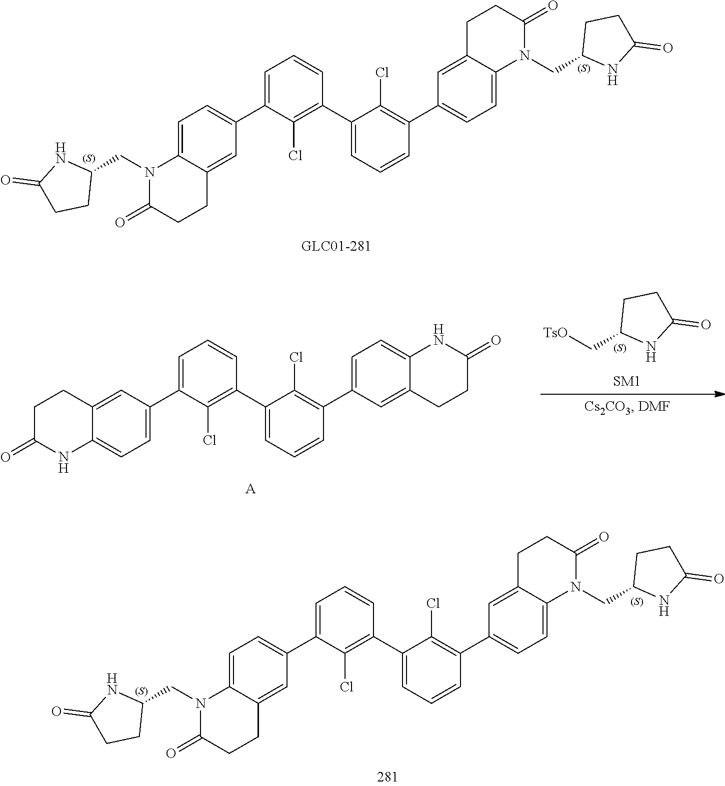

### Substrate A was Prepared Using the Same Procedure as SM5

A mixture of compound A (100 mg, 1 eq.), Cs₂CO₃ (253 mg, 4.0 eq.), SM1 (157 mg, 3.0 eq.) in DMF was stirred at 40° C. for 2 h. The reaction mixture was diluted with water and extracted with EA. The combined organic layers were washed with brine, dried over Na₂SO₄, filtered, and concentrated under reduced pressure. The residue was purified by column (DCM/MeOH=20/1) to give the title Compound 281 (98 mg, yield: 71%). ¹H NMR (500 MHz, Chloroform-d) δ 7.46-7.37 (m, 3H), 7.36 (d, J=2.1 Hz, 1H), 7.33 (dd, J=7.4, 2.0 Hz, 1H), 7.08 (d, J=8.5 Hz, 1H), 5.89 (s, 1H), 4.20-3.98 (m, 3H), 2.99 (t, J=7.5 Hz, 2H), 2.83-2.66 (m, 2H), 2.40-2.30 (m, 2H), 2.10-1.85 (m, 2H).

LCMS (ESI): calculated for C₄₀H₃₆Cl₂N₄O₄, [M+H]⁺: 707.22, found: 707.43.

### 9B. The Following Compound could be Prepared Using Different Bromide Substrate

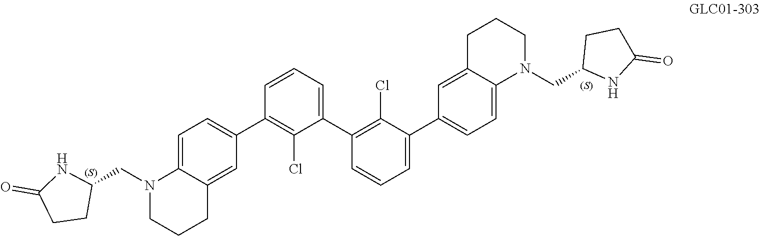

1H NMR (500 MHz, DMSO-d6) δ 7.93 (s, 2H), 7.80 (d, J=8.8 Hz, 2H), 7.75 (dd, J=8.7, 2.1 Hz, 2H), 7.71 (s, 2H), 7.60-7.54 (m, 4H), 7.51-7.47 (m, 2H), 3.91-3.82 (m, 2H), 3.61-3.53 (m, 4H), 3.41-3.33 (m, 4H), 2.85-2.76 (m, 4H), 2.39-2.26 (m, 4H), 2.25-2.06 (m, 6H), 1.92-1.84 (m, 2H) LCMS (ESI): calculated for C₄₀H₄₀Cl₂N₄O₂; [M+H]+: 679.25, found: 679.45.

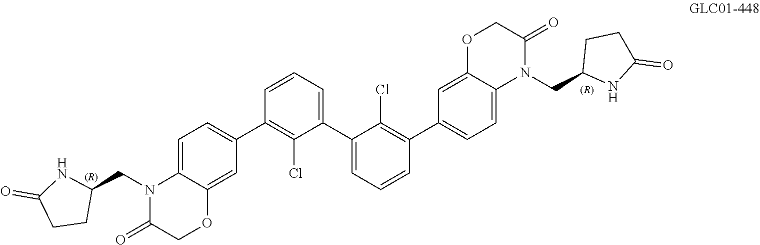

¹H NMR (500 MHz, DMSO-d₆) δ 7.78 (s, 2H), 7.50 (t, J=7.5 Hz, 2H), 7.45 (dd, J=7.7, 1.7 Hz, 2H), 7.40 (d, J=7.4 Hz, 2H), 7.34 (d, J=8.4 Hz, 2H), 7.14 (d, J=8.3 Hz, 2H), 7.10 (d, J=2.0 Hz, 2H), 4.71 (s, 4H), 4.06-3.89 (m, 6H), 2.34-2.24 (m, 2H), 2.20-2.07 (m, 4H), 1.84-1.72 (m, 2H).

LCMS (ESI): calculated for C₃₈H₃₂Cl₂N₄O₆; [M+H]⁺: 711.17, found: 711.47.

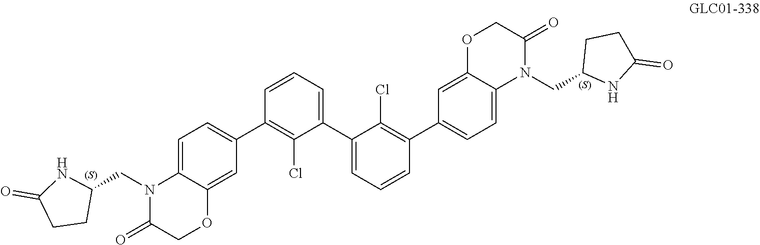

¹H NMR (500 MHz, DMSO-d₆) δ 7.78 (s, 2H), 7.50 (t, J=7.5 Hz, 2H), 7.45 (dd, J=7.8, 1.8 Hz, 2H), 7.40 (d, J=7.5 Hz, 2H), 7.34 (d, J=8.4 Hz, 2H), 7.14 (dd, J=8.6, 1.8 Hz, 2H), 7.10 (d, J=2.1 Hz, 2H), 4.71 (s, 4H), 4.07-3.88 (m, 6H), 2.35-2.24 (m, 2H), 2.21-2.04 (m, 4H), 1.84-1.73 (m, 2H).

LCMS (ESI): calculated for C₃₈H₃₂Cl₂N₄O₆; [M+H]⁺: 711.17, found: 711.50.

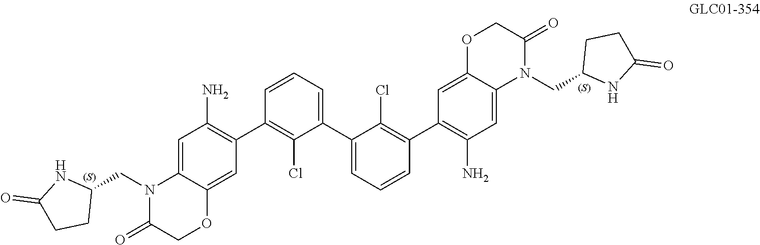

¹H NMR (500 MHz, DMSO-d₆) δ 7.78 (s, 2H), 7.50 (t, J=7.5 Hz, 2H), 7.45 (dd, J=7.8, 1.8 Hz, 2H), 7.40 (d, J=7.5 Hz, 2H), 7.34 (s, 2H), 7.10 (s, 2H), 4.71 (s, 4H), 4.07-3.88 (m, 6H), 2.35-2.24 (m, 2H), 2.21-2.04 (m, 4H), 1.84-1.73 (m, 2H).

LCMS (ESI): calculated for C₃₈H₃₄Cl₂N₆O₆; [M+H]⁺: 741.19, found: 741.50.

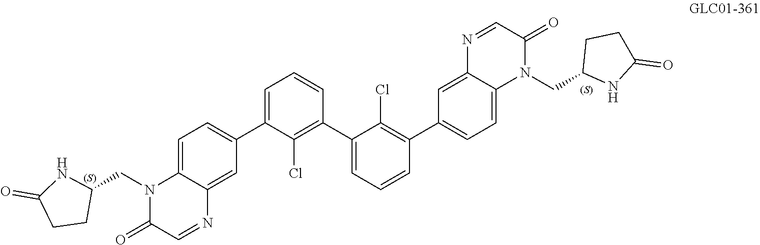

¹H NMR (500 MHz, DMSO-d₆) δ 8.29 (s, 1H), 7.93 (t, J=1.7 Hz, 1H), 7.80 (d, J=8.9 Hz, 1H), 7.75 (dd, J=8.7, 2.1 Hz, 1H), 7.70 (s, 1H), 7.57 (d, J=3.9 Hz, 2H), 7.49 (dd, J=5.6, 3.8 Hz, 1H), 4.44-4.37 (m, 1H), 4.27-4.20 (m, 1H), 4.11-4.03 (m, 1H), 2.42-2.30 (m, 1H), 2.24-2.16 (m, 1H), 2.03-1.95 (m, 1H), 1.891-1.84 (m, 1H).

LCMS (ESI): calculated for C₃₈H₃₀Cl₂N₆O₄; [M+H]⁺: 705.18, found: 705.52.

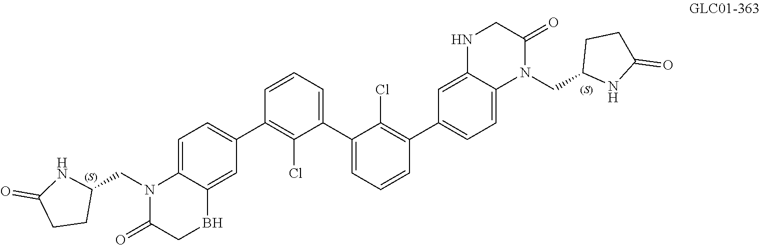

¹H NMR (500 MHz, DMSO-d₆) δ 8.29 (s, 2H), 7.93 (s, 2H), 7.80 (d, J=8.8 Hz, 2H), 7.75 (dd, J=8.7, 2.1 Hz, 2H), 7.71 (s, 2H), 7.60-7.54 (m, 4H), 7.51-7.47 (m, 2H), 4.41 (dd, J=14.0, 8.6 Hz, 2H), 4.23 (dd, J=13.8, 5.3 Hz, 2H), 4.09-4.02 (m, 2H), 2.60-2.54 (m, 2H), 2.41-2.32 (m, 3H), 2.25-2.06 (m, 5H), 1.92-1.84 (m, 2H).

LCMS (ESI): calculated for C₃₈H₃₄Cl₂N₆O₄; [M+H]⁺: 709.20, found: 709.44.

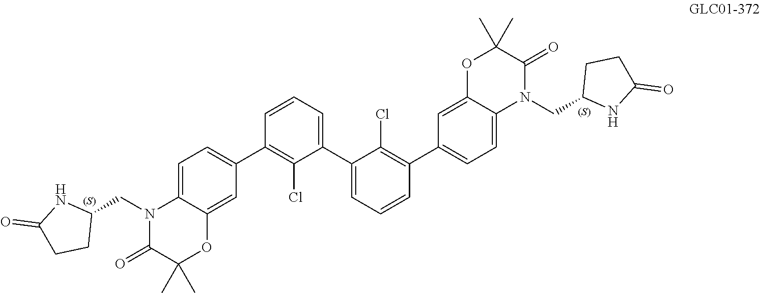

¹H NMR (500 MHz, DMSO-d₆) δ 8.13 (s, 2H), 7.86 (s, 2H), 7.58-7.53 (m, 6H), 7.46 (dd, J=5.9, 3.6 Hz, 2H), 4.21-4.15 (m, 2H), 4.05 (dd, J=13.2, 6.6 Hz, 2H), 4.01-3.96 (m, 2H), 2.20 (q, J=10.8 Hz, 4H), 2.08 (dd, J=11.3, 3.9 Hz, 4H), 1.53 (s, 6H), 1.48 (s, 6H).

LCMS (ESI): calculated for C₄₂H₄₀Cl₂N₄O₆; [M+H]⁺: 767.24, found: 767.50.

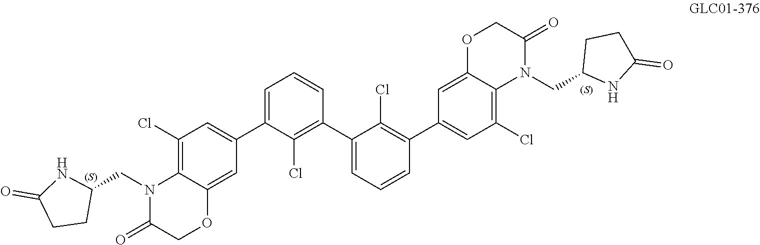

¹H NMR (500 MHz, DMSO-d₆) δ 7.79-7.71 (m, 2H), 7.55-7.49 (m, 2H), 7.48-7.41 (m, 4H), 7.38 (td, J=7.2, 1.7 Hz, 2H), 7.06 (d, J=2.6 Hz, 1H), 7.01 (d, J=1.9 Hz, 1H), 4.73 (s, 4H), 4.04-3.88 (m, 6H), 2.33-1.94 (m, 6H), 1.78 (d, J=13.0 Hz, 2H).

LCMS (ESI): calculated for C₃₈H₃₀Cl₄N₄O₆; [M+H]⁺: 779.10, found: 779.41.

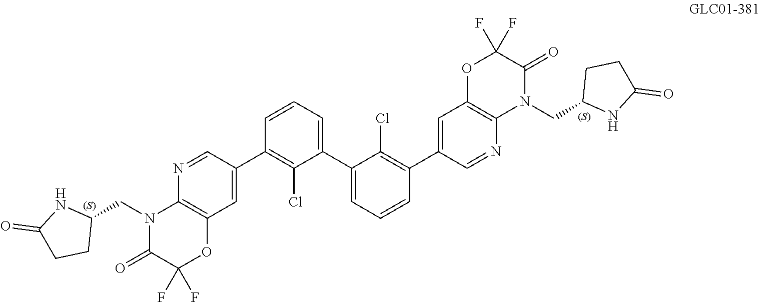

¹H NMR (400 MHz, DMSO-d₆) δ 7.94 (s, 2H), 7.62 (d, J=8 Hz, 2H), 7.56-7.49 (m, 6H), 7.45 (s, 2H), 4.22 (m, 2H), 4.06 (dd, J=4 Hz, 2H), 3.95 (m, 2H), 2.32 (m, 2H), 2.20 (m, 2H), 2.10 (m, 2H) 1.80 (m, 2H).

LCMS (ESI): calculated for C₃₆H₂₆F₄Cl₂N₆O₆; [M+H]⁺: 785.12, found: 785.12.

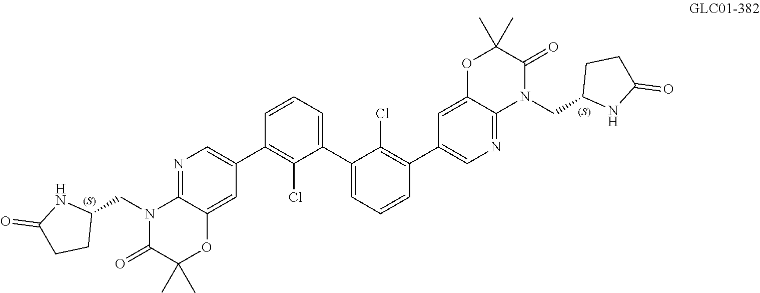

¹H NMR (500 MHz, DMSO-d₆) δ 8.13 (s, 2H), 7.86 (s, 2H), 7.58-7.53 (m, 6H), 7.46 (dd, J=5.9, 3.6 Hz, 2H), 4.21-4.15 (m, 2H), 4.05 (dd, J=13.2, 6.6 Hz, 2H), 4.01-3.96 (m, 2H), 2.20 (q, J=10.8 Hz, 4H), 2.08 (dd, J=11.3, 3.9 Hz, 4H), 1.53 (s, 6H), 1.48 (s, 6H).

LCMS (ESI): calculated for C₄₀H₃₈Cl₂N₆O₆; [M+H]⁺: 769.23, found: 769.50.

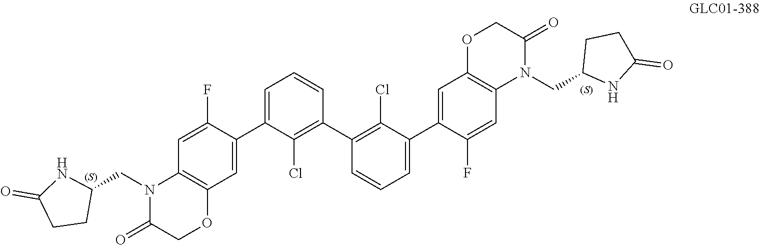

¹HNMR (500 MHz, DMSO-d₆) δ 7.76 (s, 2H), 7.53 (t, J=7.4 Hz, 3H), 7.48-7.42 (m, 4H), 7.31 (t, J=10.8 Hz, 2H), 7.05 (d, J=6.9 Hz, 2H), 4.70 (s, 4H), 4.05-3.87 (m, 6H), 2.33-2.23 (m, 2H), 2.20-2.04 (m, 4H), 1.77 (dd, J=13.6, 7.7 Hz, 2H).

LCMS (ESI): calculated for C₃₈H₃₀Cl₂F₂N₄O₆; [M+H]⁺: 747.15, found: 747.10.

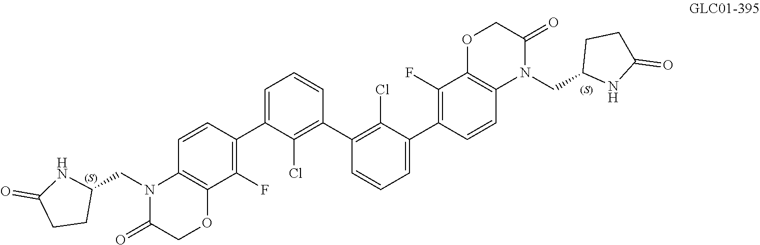

¹H NMR (500 MHz, DMSO-d₆) δ 7.79 (d, J=2.9 Hz, 2H), 7.54 (t, J=7.5 Hz, 2H), 7.46 (d, J=7.1 Hz, 4H), 7.19 (d, J=8.6 Hz, 2H), 7.04 (t, J=7.8 Hz, 2H), 4.81 (d, J=14.7 Hz, 4H), 4.08-4.01 (m, 2H), 3.93 (d, J=12.0 Hz, 4H), 2.34-2.23 (m, 2H), 2.21-2.04 (m, 4H), 1.77 (dd, J=13.2, 8.2 Hz, 2H).

LCMS (ESI): calculated for C₃₈H₃₀Cl₂F₂N₄O₆; [M+H]⁺: 747.15, found: 747.11.

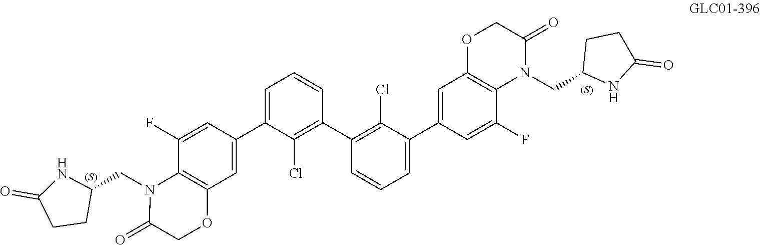

¹H NMR (500 MHz, DMSO-d₆) δ 7.88 (s, 2H), 7.51 (dt, J=14.3, 7.2 Hz, 4H), 7.42 (d, J=7.0 Hz, 2H), 7.15 (d, J=14.0 Hz, 2H), 7.04 (s, 2H), 4.74-4.67 (m, 4H), 4.10 (dd, J=14.3, 7.5 Hz, 2H), 3.98-3.85 (m, 4H), 2.19 (t, J=9.6 Hz, 2H), 2.15-2.06 (m, 4H), 1.70 (dd, J=16.5, 7.0 Hz, 2H).

LCMS (ESI): calculated for C₃₈H₃₀Cl₂F₂N₄O₆; [M+H]⁺: 747.15, found: 747.15.

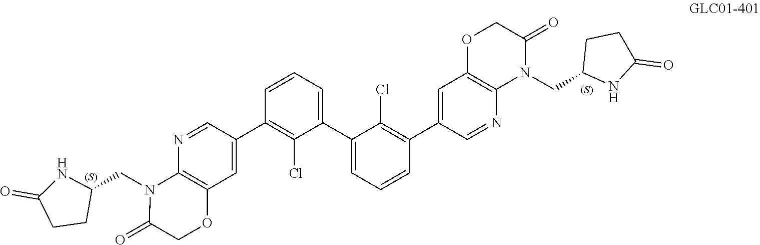

¹H NMR (400 MHz, DMSO-d₆) δ 8.61 (s, 2H), 7.92 (s, 2H), 7.81 (s, 2H), 7.62-7.75 (m, 6H), 4.78 (s, 4H), 4.22 (m, 2H), 4.06 (dd, J=4 Hz, 2H), 3.95 (m, 2H), 2.32 (m, 2H), 2.20 (m, 2H), 2.10 (m, 2H) 1.80 (m, 2H).

LCMS (ESI): calculated for C₃₆H₃₀Cl₂N₆O₆; [M+H]⁺: 713.16, found: 713.16.

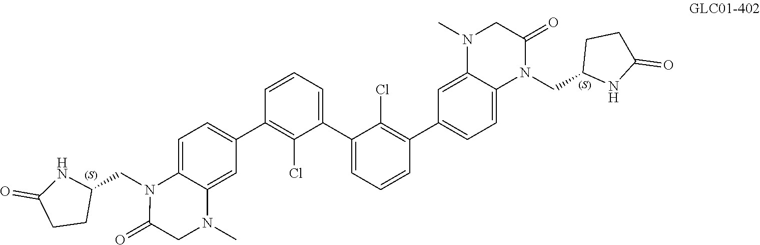

¹H NMR (500 MHz, DMSO-d₆) δ 7.93 (s, 2H), 7.80 (d, J=8.8 Hz, 2H), 7.75 (dd, J=8.7, 2.1 Hz, 2H), 7.71 (s, 2H), 7.60-7.54 (m, 4H), 7.51-7.47 (m, 2H), 4.41 (dd, J=14.0, 8.6 Hz, 2H), 4.23 (dd, J=13.8, 5.3 Hz, 2H), 4.09-4.02 (m, 2H), 3.50 (s, 6H), 2.60-2.54 (m, 2H), 2.41-2.32 (m, 3H), 2.25-2.06 (m, 5H), 1.92-1.84 (m, 2H).

LCMS (ESI): calculated for C₄₀H₃₈Cl₂N₆O₄; [M+H]⁺: 737.23, found: 737.52

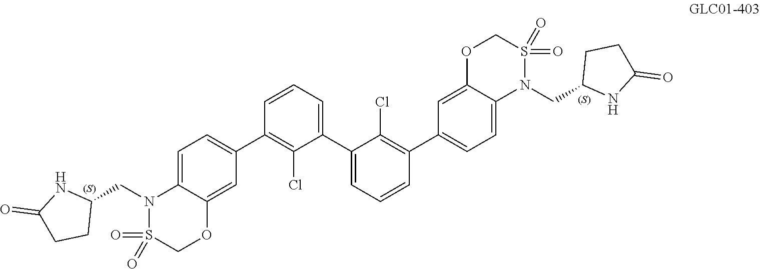

¹H NMR (500 MHz, DMSO-d₆) δ 7.81 (s, 2H), 7.52 (t, J=7.5 Hz, 2H), 7.46 (dd, J=7.6, 1.7 Hz, 2H), 7.42 (dd, J=7.4, 1.9 Hz, 2H), 7.31 (d, J=8.5 Hz, 2H), 7.25-7.20 (m, 2H), 7.19 (d, J=2.1 Hz, 2H), 5.53 (s, 4H), 3.99-3.88 (m, 2H), 3.78 (d, J=5.6 Hz, 4H), 2.32-2.24 (m, 2H), 2.23-2.09 (m, 4H), 1.90-1.80 (m, 2H).

LCMS (ESI): calculated for C₃₆H₃₂Cl₂N₄O₈S₂; [M+H]⁺: 783.10, found: 783.42.

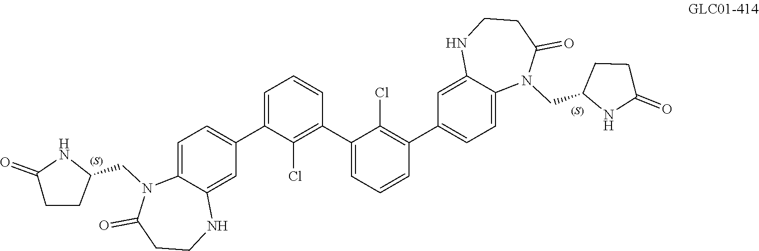

¹H NMR (500 MHz, DMSO-d₆) δ 7.50 (t, J=7.5 Hz, 1H), 7.48-7.43 (m, 1H), 7.41-7.32 (m, 3H), 7.12 (d, J=2.1 Hz, 1H), 7.07 (dt, J=8.3, 1.6 Hz, 1H), 5.38 (s, 1H), 3.91-3.74 (m, 2H), 3.68-3.50 (m, 3H), 2.48-2.32 (m, 2H), 2.15-1.93 (m, 4H).

LCMS (ESI): calculated for C₄₀H₃₈Cl₂N₆O₄; [M+H]⁺: 737.24, found: 737.57.

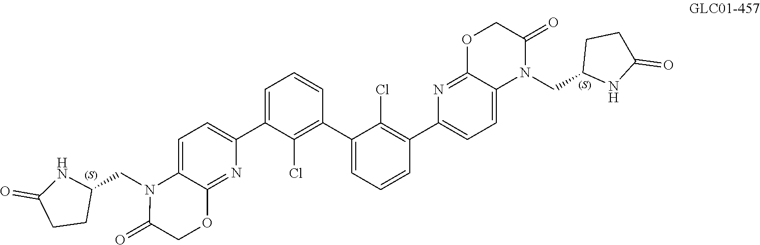

¹H NMR (500 MHz, DMSO-d₆) δ 7.78-7.73 (m, 2H), 7.58 (dd, J=7.7, 1.9 Hz, 1H), 7.54 (t, J=7.6 Hz, 1H), 7.43 (dd, J=7.4, 1.9 Hz, 1H), 7.38 (d, J=8.1 Hz, 1H), 4.91 (d, J=1.4 Hz, 2H), 4.06-4.01 (m, 1H), 3.96-3.90 (m, 2H), 2.37-2.24 (m, 2H), 2.16-2.05 (m, 2H).

LCMS (ESI): calculated for C₃₆H₃₀Cl₂N₆O₆; [M+H]⁺: 713.17, found: 713.48.

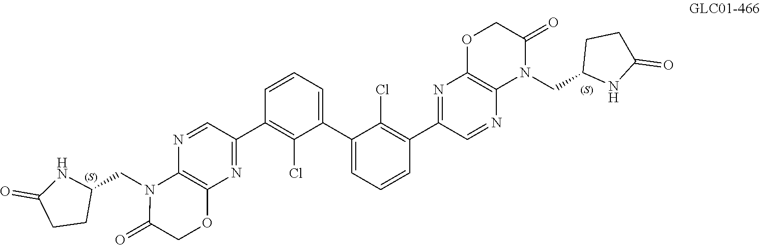

¹H NMR (400 MHz, DMSO-d₆) δ 8.71 (s, 2H), 7.92 (s, 2H), 7.62-7.75 (m, 6H), 4.78 (s, 4H), 4.22 (m, 2H), 4.06 (dd, J=4 Hz, 2H), 3.95 (m, 2H), 2.32 (m, 2H), 2.20 (m, 2H), 2.10 (m, 2H) 1.80 (m, 2H).

LCMS (ESI): calculated for C₃₄H₂₈Cl₂N₈O₆; [M+H]⁺: 715.15, found: 715.15.

### Example 10 (Preparation of Same Core Different Side)

### 10A. Preparation of 2,2′-((2,2′-dichloro-[1,1′-biphenyl]-3,3′-diyl)bis(3-oxo-2,3-dihydro-4H-benzo[b][1,4]oxazine-7,4-diyl))diacetonitrile

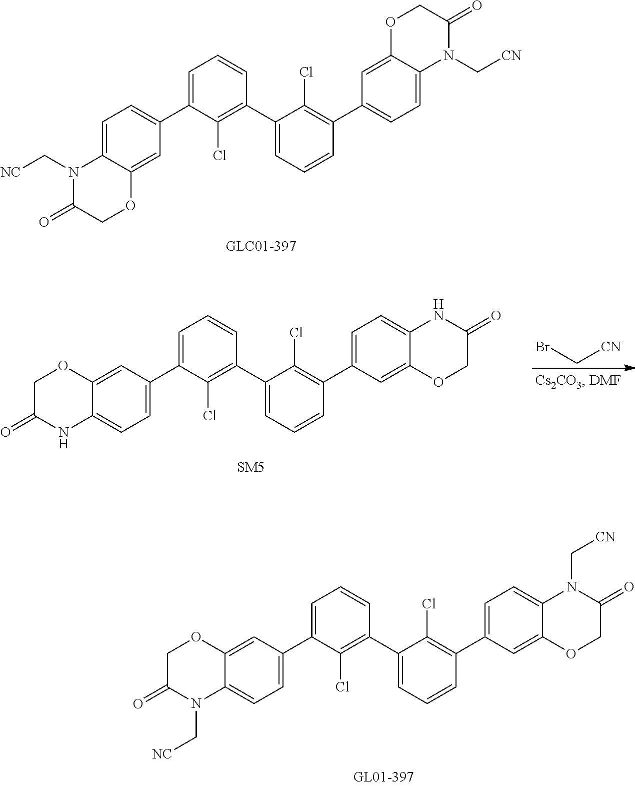

A mixture of compound SM5 (100 mg, 1 eq.), Cs2CO3 (251 mg, 4.0 eq.), bromoacetonitrile (69 mg, 3.0 eq.) in DMF was stirred at 40° C. for 2 h. The reaction mixture was diluted with water and extracted with EA. The combined organic layers were washed with brine, dried over Na2SO4, filtered, and concentrated under reduced pressure. The residue was purified by column (DCM/MeOH=20/1) to give the title Compound 397 (92 mg, yield: 67.6%).

1H NMR (500 MHz, DMSO-d6) δ 7.54-7.46 (m, 2H), 7.44-7.38 (m, 2H), 7.25 (dd, J=8.3, 2.0 Hz, 1H), 7.18 (d, J=2.0 Hz, 1H), 5.15 (s, 2H), 4.84 (s, 2H).

LCMS (ESI): calculated for C32H20Cl2N4O4; [M+H]+: 595.09, found: 595.40.

### 10B. Following the Procedure Above, the Compound could be Prepared from Different Bromide Side Chain

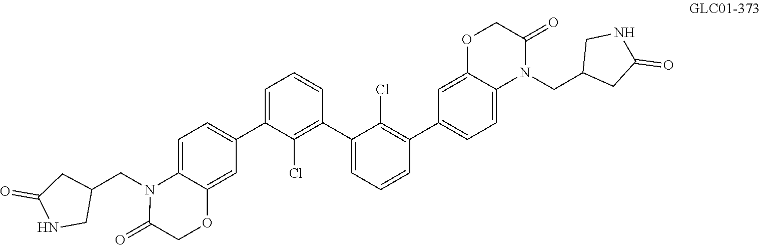 [SMILES: O=C1CC(CN2C(=O)COc3cc(-c4cccc(-c5cccc(-c6ccc7c(c6)OCC(=O)N7CC6CNC(=O)C6)c5Cl)c4Cl)ccc32)CN1]

1H NMR (500 MHz, DMSO-d6) δ 7.55 (br s, 1H), 7.52-7.45 (m, 2H), 7.41-7.36 (m, 2H), 7.16 (dd, J=8.4, 2.0 Hz, 1H), 7.12 (d, J=2.0 Hz, 1H), 4.73 (s, 2H), 4.11-3.98 (m, 2H), 3.34-3.28 (m, 1H), 3.06-2.99 (m, 1H), 2.83-2.74 (m, 1H), 2.30-2.22 (m, 1H), 2.02-1.95 (m, 1H).

LCMS (ESI): calculated for C38H32Cl2N₄O6; [M+H]+: 711.18, found: 711.47.

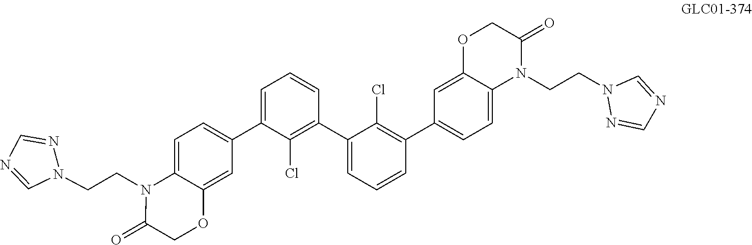 [SMILES: O=C1COc2cc(-c3cccc(-c4cccc(-c5ccc6c(c5)OCC(=O)N6CCn5cncn5)c4Cl)c3Cl)ccc2N1CCn1cncn1]

1H NMR (500 MHz, DMSO-d6) δ 8.53 (d, J=1.2 Hz, 1H), 7.95 (d, J=1.5 Hz, 1H), 7.52-7.47 (m, 1H), 7.44 (dd, J=7.7, 1.8 Hz, 1H), 7.39 (dd, J=7.4, 1.7 Hz, 1H), 7.12-7.08 (m, 3H), 4.67 (s, 2H), 4.49 (t, J=6.0 Hz, 2H), 4.33 (t, J=6.1 Hz, 2H).

LCMS (ESI): calculated for C36H28Cl2N8O4; [M+H]+: 707.17, found: 707.43.

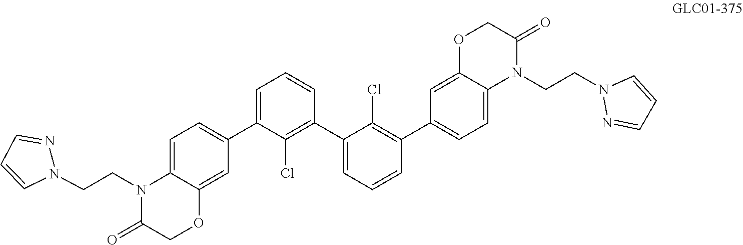 [SMILES: O=C1COc2cc(-c3cccc(-c4cccc(-c5ccc6c(c5)OCC(=O)N6CCn5cccn5)c4Cl)c3Cl)ccc2N1CCn1cccn1]

1H NMR (500 MHz, DMSO-d6) δ 7.74 (d, J=1.9 Hz, 1H), 7.52-7.47 (m, 1H), 7.46-7.41 (m, 2H), 7.39 (dd, J=7.4, 1.8 Hz, 1H), 7.09 (d, J=1.9 Hz, 1H), 7.06 (dd, J=8.4, 2.0 Hz, 1H), 6.94 (d, J=8.5 Hz, 1H), 6.18 (t, J=2.0 Hz, 1H), 4.68 (d, J=1.4 Hz, 2H), 4.40 (t, J=6.2 Hz, 2H), 4.28 (t, J=6.2 Hz, 2H).

LCMS (ESI): calculated for C38H30Cl2N6O4; [M+H]+: 705.18, found: 705.42.

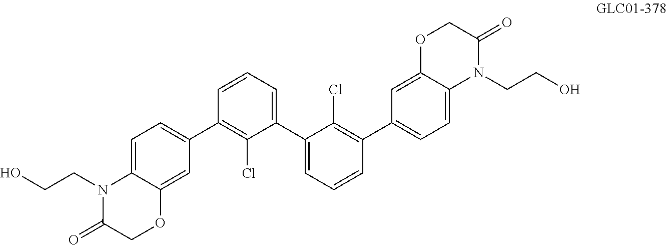 [SMILES: O=C1COc2cc(-c3cccc(-c4cccc(-c5ccc6c(c5)OCC(=O)N6CCO)c4Cl)c3Cl)ccc2N1CCO]

1H NMR (500 MHz, DMSO-d6) δ 7.50 (t, J=7.5 Hz, 2H), 7.45 (dd, J=7.7, 1.9 Hz, 2H), 7.42-7.36 (m, 4H), 7.15 (dd, J=8.2, 2.0 Hz, 2H), 7.09 (d, J=2.0 Hz, 2H), 4.93 (t, J=5.7 Hz, 2H), 4.69 (s, 4H), 4.00 (t, J=6.1 Hz, 4H), 3.65-3.59 (m, 4H).

LCMS (ESI): calculated for C32H26Cl2N2O6; [M+H]+: 605.12, found: 605.50.

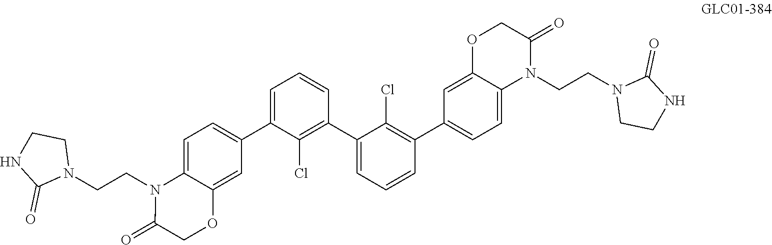 [SMILES: O=C1NCCN1CCN1C(=O)COc2cc(-c3cccc(-c4cccc(-c5ccc6c(c5)OCC(=O)N6CCN5CCNC5=O)c4Cl)c3Cl)ccc21]

1H NMR (500 MHz, DMSO-d6) δ 7.52-7.45 (m, 2H), 7.41-7.37 (m, 2H), 7.18 (dd, J=8.4, 2.0 Hz, 1H), 7.10 (d, J=2.0 Hz, 1H), 4.68 (s, 2H), 4.06 (t, J=6.5 Hz, 2H), 3.43 (dd, J=9.0, 6.8 Hz, 2H), 3.31 (t, J=6.5 Hz, 2H), 3.17 (dd, J=8.9, 6.8 Hz, 2H).

LCMS (ESI): calculated for C38H34Cl2N6O6; [M+H]+: 741.20, found: 741.50.

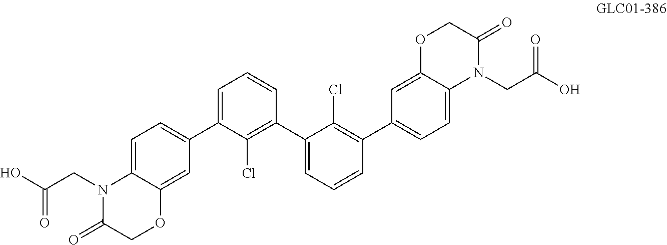 [SMILES: O=C(O)CN1C(=O)COc2cc(-c3cccc(-c4cccc(-c5ccc6c(c5)OCC(=O)N6CC(=O)O)c4Cl)c3Cl)ccc21]

1H NMR (500 MHz, DMSO-d6) δ 7.48 (t, J=7.6 Hz, 1H), 7.43 (dd, J=7.6, 1.9 Hz, 1H), 7.35 (dd, J=7.4, 1.8 Hz, 1H), 7.07 (dd, J=8.3, 2.0 Hz, 1H), 7.03 (d, J=2.0 Hz, 1H), 6.94 (d, J=8.4 Hz, 1H), 4.65 (s, 2H), 4.16 (s, 2H).

LCMS (ESI): calculated for C32H22Cl2N2O8; [M+H]+: 633.08, found: 633.37.

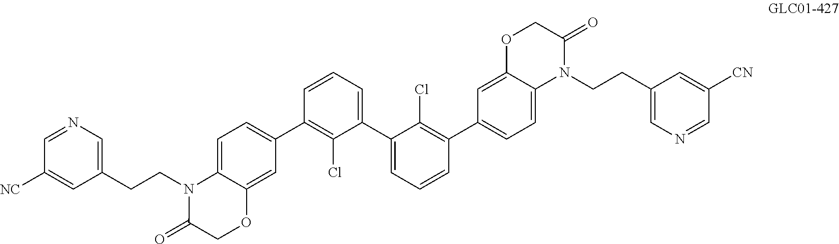 [SMILES: N#Cc1cncc(CCN2C(=O)COc3cc(-c4cccc(-c5cccc(-c6ccc7c(c6)OCC(=O)N7CCc6cncc(C#N)c6)c5Cl)c4Cl)ccc32)c1]

1H NMR (500 MHz, DMSO-d6) δ 8.87 (d, J=2.0 Hz, 2H), 8.74 (d, J=2.1 Hz, 2H), 8.28 (d, J=2.3 Hz, 2H), 7.52 (t, J=7.6 Hz, 2H), 7.46 (dd, J=7.7, 1.9 Hz, 2H), 7.44-7.39 (m, 4H), 7.16 (dd, J=8.3, 2.0 Hz, 2H), 7.12 (d, J=2.0 Hz, 2H), 4.67 (s, 4H), 4.23 (t, J=7.3 Hz, 4H), 3.01 (t, J=7.3 Hz, 4H).

LCMS (ESI): calculated for C44H30Cl2N6O4; [M+H]+: 777.17, found: 777.45.

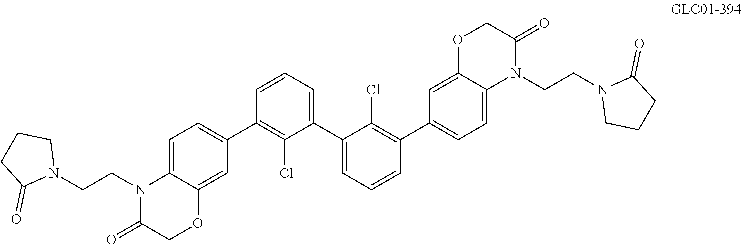 [SMILES: O=C1CCCN1CCN1C(=O)COc2cc(-c3cccc(-c4cccc(-c5ccc6c(c5)OCC(=O)N6CCN5CCCC5=O)c4Cl)c3Cl)ccc21]

1H NMR (500 MHz, DMSO-d6) δ 7.52 (t, J=7.6 Hz, 2H), 7.46 (d, J=7.8 Hz, 2H), 7.41 (d, J=7.5 Hz, 2H), 7.36 (d, J=8.4 Hz, 2H), 7.19 (d, 2H), 7.14 (d, J=2.0 Hz, 2H), 4.75 (s, 4H), 3.75-3.67 (m, 4H), 3.24-3.17 (m, 4H), 3.08-3.02 (m, 4H), 2.34-2.16 (m, 4H), 1.97-2.01 (m, 4H).

LCMS (ESI): calculated for C40H36Cl2N4O6; [M+H]+: 739.20, found: 739.50.

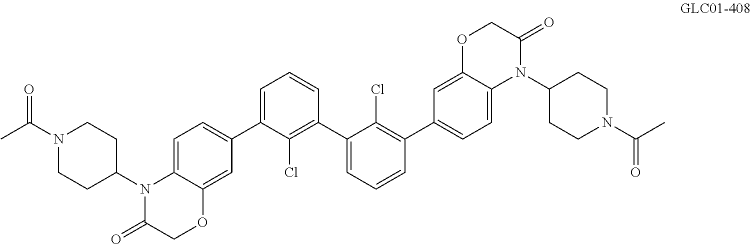 [SMILES: CC(=O)N1CCC(N2C(=O)COc3cc(-c4cccc(-c5cccc(-c6ccc7c(c6)OCC(=O)N7C6CCN(C(C)=O)CC6)c5Cl)c4Cl)ccc32)CC1]

1H NMR (500 MHz, DMSO-d6) δ 7.54-7.48 (m, 2H), 7.46 (dd, J=7.6, 1.9 Hz, 1H), 7.40 (dd, J=7.4, 1.9 Hz, 1H), 7.19 (dd, J=8.4, 2.1 Hz, 1H), 7.13 (d, J=2.0 Hz, 1H), 4.59 (s, 2H), 4.52 (d, J=13.1 Hz, 1H), 4.39 (t, J=12.3 Hz, 1H), 3.92 (d, J=13.5 Hz, 1H), 3.25-3.13 (m, 1H), 2.70-2.53 (m, 2H), 2.42-2.27 (m, 1H), 2.04 (s, 3H), 1.86-1.71 (m, 2H).

LCMS (ESI): calculated for C42H40Cl2N₄O6; [M+H]+: 767.24, found: 767.56.

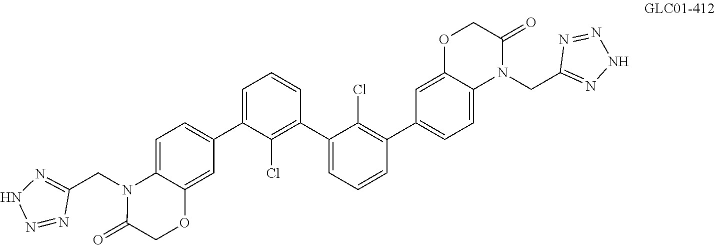 [SMILES: O=C1COc2cc(-c3cccc(-c4cccc(-c5ccc6c(c5)OCC(=O)N6Cc5nn[nH]n5)c4Cl)c3Cl)ccc2N1Cc1nn[nH]n1]

1H NMR (500 MHz, DMSO-d6) δ 7.53-7.31 (m, 4H), 7.25 (dd, J=8.3, 2.0 Hz, 1H), 7.05 (d, J=2.0 Hz, 1H), 5.36 (s, 2H), 4.77 (s, 2H).

LCMS (ESI): calculated for C32H22Cl2N10O4; [M+H]+: 681.13, found: 681.41.

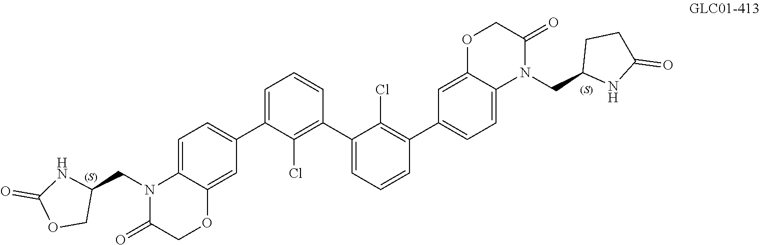

1H NMR (500 MHz, DMSO-d6) δ 7.84 (s, 1H), 7.51 (t, J=7.5 Hz, 1H), 7.46 (dd, J=7.7, 1.9 Hz, 1H), 7.42-7.38 (m, 1H), 7.36 (d, J=8.5 Hz, 1H), 7.15 (dd, J=8.3, 2.1 Hz, 1H), 7.11 (d, J=2.0 Hz, 1H), 4.72 (d, J=3.3 Hz, 2H), 4.41 (t, J=7.9 Hz, 1H), 4.20-4.08 (m, 3H), 4.02-3.94 (m, 1H).

LCMS (ESI): calculated for C36H28Cl2N₄O8; [M+H]+: 715.14, found: 715.42.

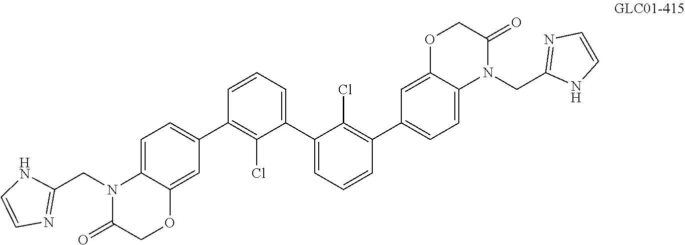 [SMILES: O=C1COc2cc(-c3cccc(-c4cccc(-c5ccc6c(c5)OCC(=O)N6Cc5ncc[nH]5)c4Cl)c3Cl)ccc2N1Cc1ncc[nH]1]

1H NMR (500 MHz, DMSO-d6) δ 7.78 (s, 2H), 7.50 (t, J=7.5 Hz, 2H), 7.45 (dd, J=7.8, 1.8 Hz, 2H), 7.40 (d, J=7.5 Hz, 2H), 7.34 (d, J=8.4 Hz, 2H), 7.14 (dd, J=8.6, 1.8 Hz, 2H), 7.10 (d, J=2.1 Hz, 2H), 7.09 (d, J=5.4, 2.8 Hz, 2H), 6.96 (d, J=2.7 Hz, 2H), 4.74 (s, 4H), LCMS (ESI): calculated for C36H26Cl2N6O4; [M+H]+: 677.14, found: 677.43.

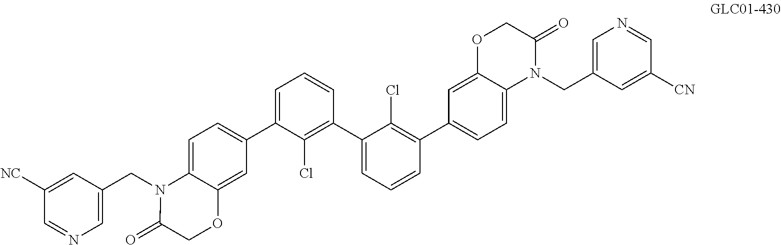 [SMILES: N#Cc1cncc(CN2C(=O)COc3cc(-c4cccc(-c5cccc(-c6ccc7c(c6)OCC(=O)N7Cc6cncc(C#N)c6)c5Cl)c4Cl)ccc32)c1]

1H NMR (500 MHz, DMSO-d6) δ 8.95 (d, J=2.0 Hz, 1H), 8.91 (d, J=2.3 Hz, 1H), 8.29 (q, J=2.4 Hz, 1H), 7.47 (dq, J=7.7, 4.2 Hz, 1H), 7.42 (dt, J=7.5, 2.4 Hz, 1H), 7.37 (dt, J=7.6, 2.3 Hz, 1H), 7.13 (d, J=4.1 Hz, 1H), 7.05 (d, J=4.2 Hz, 2H), 5.27 (d, J=4.2 Hz, 2H), 4.91 (d, J=4.2 Hz, 2H).

LCMS (ESI): calculated for C42H26Cl2N6O4; [M+H]+: 749.15, found: 749.44.

### Example 11 (Same Core Different Side)

### 11A. Preparation of 7,7′-(2,2′-dichloro-[1,1′-biphenyl]-3,3′-diyl)bis(4-(((S)-pyrrolidin-2-yl)methyl)-2H-benzo[b][1,4]oxazin-3(4H)-one)

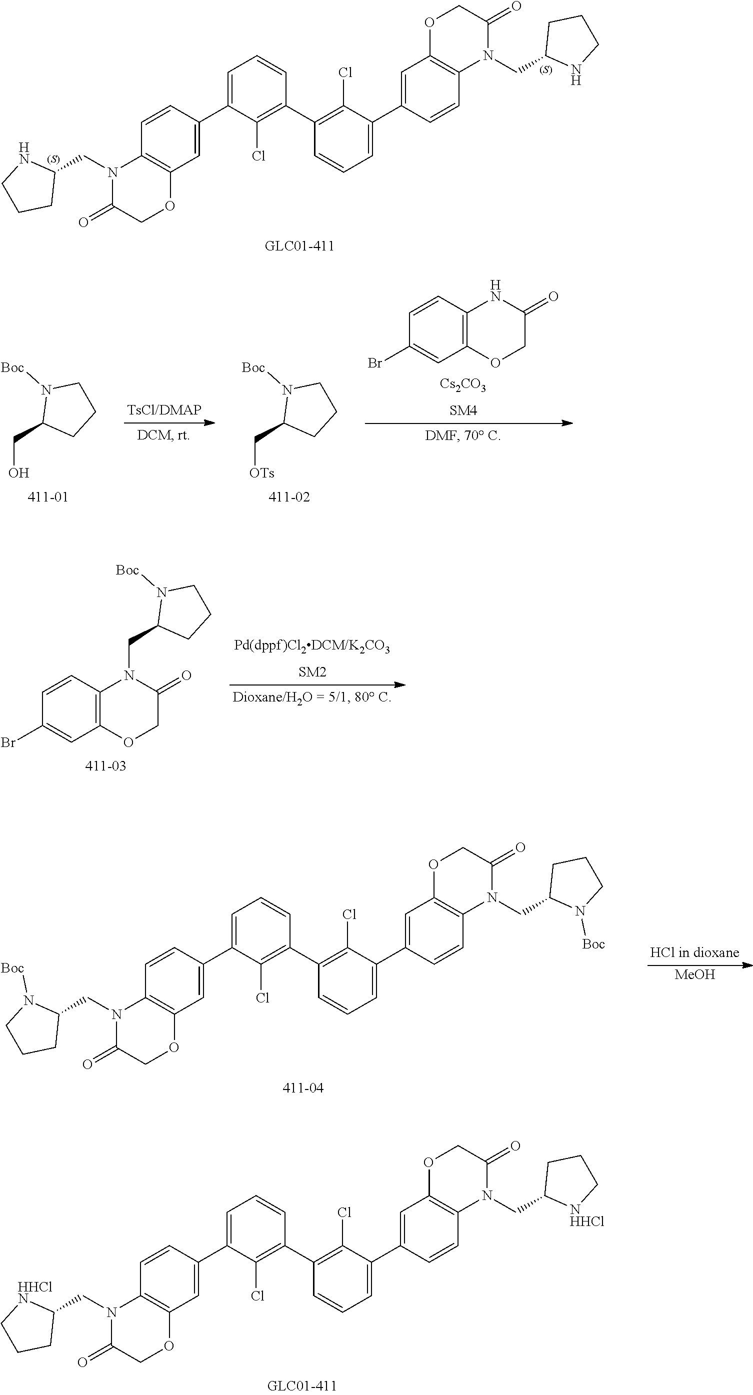

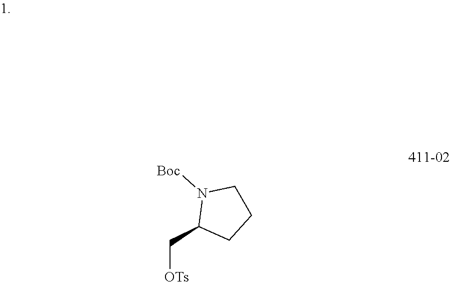 [SMILES: Cc1ccc(S(=O)(=O)OC[C@@H]2CCCN2C(=O)OC(C)(C)C)cc1]

To a stirred solution of 411-01 (500 mg, 2.49 mmol, 1.0 eq) in DCM (5 ml) were added TosCl (567 mg, 2.98 mmol, 1.2 eq) and TEA (502 mg, 4.97 mmol, 2.0 eq), followed by added DMAP (30 mg, 0.249 mmol, 0.1 eq) at room temperature and stirred for 4 h. Water and DCM was added to the reaction and separated, the organic phase was dried over anhydrous sodium sulfate, filtered, the filtrate was concentrated under vacuum. The residue (950 mg, yield: 107%) was used directly in the next step.

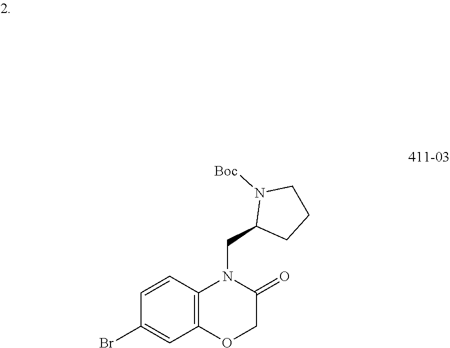 [SMILES: CC(C)(C)OC(=O)N1CCC[C@H]1CN1C(=O)COc2cc(Br)ccc21]

To a stirred solution of SM4 (117 mg, 0.51 mmol, 1.0 eq) in DMF (2 ml) were added 411-02 (219 mg, 0.62 mmol, 1.2 eq) and CS2CO3 (337 mg, 1.03 mmol, 2.0 eq) at ambient temperature. Then the resulting mixture was stirred at 70° C. overnight. The reaction was quenched with water, extracted with EA. The combined organic phase was dried over Na2SO4, filtered, the filtrate was concentrated under vacuum. The residue was purified by prep-TLC (PE/EA=4/1) to afford desired product (105 mg, yield: 50%) as a light yellow oil.

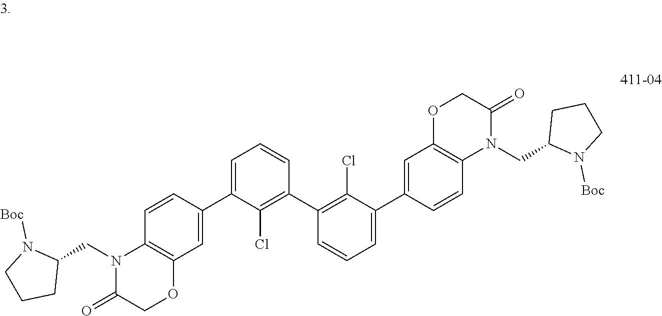 [SMILES: CC(C)(C)OC(=O)N1CCC[C@H]1CN1C(=O)COc2cc(-c3cccc(-c4cccc(-c5ccc6c(c5)OCC(=O)N6C[C@@H]5CCCN5C(=O)OC(C)(C)C)c4Cl)c3Cl)ccc21]

To a solution of 411-03 (105 mg, 0.26 mmol, 2.2 eq.) and SM2 (55 mg, 0.12 mmol, 1.0 eq) in dioxane (2.5 ml) and H₂O (0.5 ml) were added Pd (dppf) Cl₂·DCM (19 mg, 0.024 mmol, 0.2 eq), K₂CO₃ (64 mg, 0.48 mmol, 4.0 equiv) under N₂ atmosphere. Then the final mixture was heated at 80° C. for stirring 2 h. Cooled down to room temperature, the water was added and extracted with EA for twice, the combined organic phase was washed with brine and dried over anhydrous sodium sulfate, filtered, the filtrate was concentrated under vacuum. The residue was purified by prep-TLC (DCM/MeOH=15/1) to afford desired product (50 mg, yield: 49%) as a white solid.

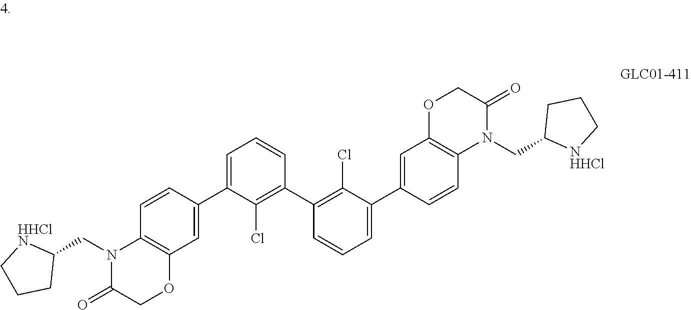

To a stirred solution of 411-03 (50 mg, 0.056 mmol, 1.0 eq) in MeOH (1 ml) was added HCl in dioxane (1 ml, 4M), then the solution was stirred at room temperature for 2 h. After the completion of the reaction, the solvent was removed by reduced pressure and dried under lyophilization to afford desired product (36 mg, yield: 95%).

1H NMR (500 MHz, DMSO-d6) δ 9.61 (s, 2H), 8.72 (s, 2H), 7.52 (t, J=7.5 Hz, 2H), 7.48-7.43 (m, 4H), 7.41 (d, J=7.4 Hz, 2H), 7.19-7.12 (m, 4H), 4.78 (t, J=8.6 Hz, 4H), 4.35 (dd, J=15.0, 7.8 Hz, 2H), 4.24 (dd, J=14.9, 5.0 Hz, 2H), 3.71 (s, 2H), 3.28 (s, 2H), 3.11 (s, 2H), 2.17 (d, J=6.7 Hz, 2H), 2.02-1.88 (m, 4H), 1.71 (dt, J=18.8, 9.3 Hz, 2H).

LCMS (ESI): calculated for C₃₈H₃₆Cl₂N₄O₄ (free form); [M+H]+: 683.2, found: 683.2.

### 11B. The Following Compounds May Also be Prepared

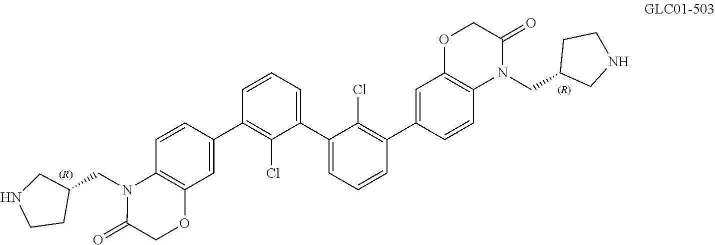

1H NMR (500 MHz, DMSO-d6) δ 9.26 (s, 4H), 7.51 (t, J=7.5 Hz, 2H), 7.48-7.42 (m, 4H), 7.40 (d, J=7.3 Hz, 2H), 7.17 (d, J=8.6 Hz, 2H), 7.13 (s, 2H), 4.73 (d, J=12.0 Hz, 4H), 4.08 (ddd, J=41.2, 14.4, 7.4 Hz, 4H), 3.32-3.23 (m, 4H), 3.15-3.05 (m, 2H), 2.98-2.89 (m, 2H), 2.66 (dd, J=15.2, 7.7 Hz, 2H), 2.07-1.99 (m, 2H), 1.69 (dq, J=16.8, 8.4 Hz, 2H).

LCMS (ESI): calculated for C₃₈H₃₆Cl₂N₄O₄ (free form); [M+H]+: 683.2, found: 683.2.

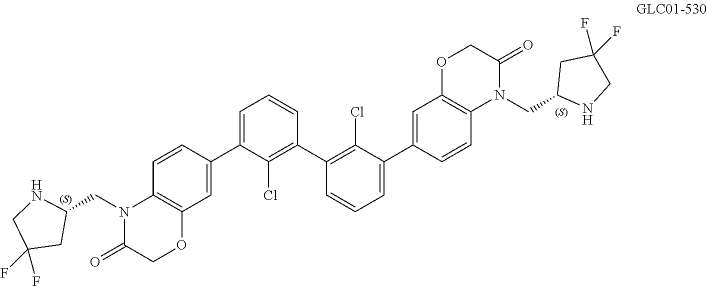

1H NMR (500 MHz, DMSO-d6) δ 10.35 (s, 2H), 9.63 (s, 2H), 7.54-7.45 (m, 6H), 7.41 (d, J=7.3 Hz, 2H), 7.16 (d, J=8.6 Hz, 2H), 7.14 (s, 2H), 4.81-4.72 (m, 4H), 4.45 (dd, J=15.2, 7.8 Hz, 2H), 4.28 (dd, J=15.2, 4.7 Hz, 2H), 4.07 (s, 2H), 3.84 (dd, J=24.5, 11.5 Hz, 2H), 3.73-3.54 (m, 4H), 2.92 (d, J=14.7 Hz, 2H).

LCMS (ESI): calculated for C₃₈H₃₂Cl₂F₄N₄O₄ (free form); [M+H]+: 755.2, found: 755.2

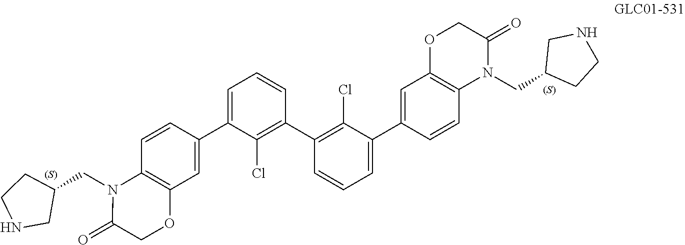

1H NMR (500 MHz, DMSO-d6) δ 9.25 (s, 4H), 7.51 (t, J=7.5 Hz, 2H), 7.48-7.42 (m, 4H), 7.40 (d, J=7.3 Hz, 2H), 7.17 (d, J=8.5 Hz, 2H), 7.13 (s, 2H), 4.74 (s, 4H), 4.08 (ddd, J=41.0, 14.5, 7.4 Hz, 4H), 3.31-3.22 (m, 4H), 3.11 (dd, J=11.9, 5.8 Hz, 2H), 2.94 (td, J=13.5, 6.5 Hz, 2H), 2.66 (dd, J=15.2, 7.6 Hz, 2H), 2.04 (dt, J=12.6, 6.4 Hz, 2H), 1.69 (dq, J=16.7, 8.3 Hz, 2H).

LCMS (ESI): calculated for C₃₈H₃₆Cl₂N₄O₄ (free form); [M+H]+: 683.2, found: 683.2.

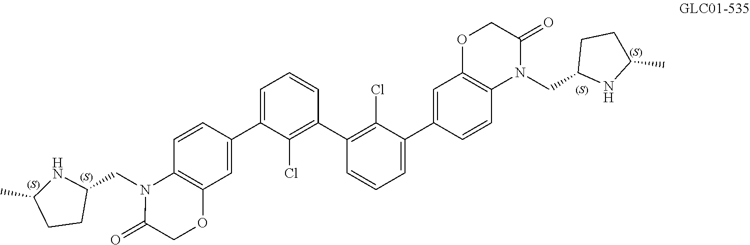

1H NMR (500 MHz, DMSO-d6) δ 9.72 (s, 2H), 8.51 (d, J=9.4 Hz, 2H), 7.52 (t, J=7.5 Hz, 2H), 7.47-7.40 (m, 6H), 7.16 (d, J=8.5 Hz, 2H), 7.14 (s, 2H), 4.77 (s, 4H), 4.42 (dd, J=15.4, 8.2 Hz, 2H), 4.19 (dd, J=15.0, 4.3 Hz, 2H), 3.75 (s, 2H), 3.51 (s, 2H), 2.21 (d, J=8.5 Hz, 2H), 2.14-2.06 (m, 2H), 1.83 (dd, J=13.9, 8.4 Hz, 2H), 1.67-1.59 (m, 2H), 1.33 (d, J=6.4 Hz, 6H).

LCMS (ESI): calculated for C₄₀H₄₀Cl₂N₄O₄ (free form); [M+H]+: 711.2, found: 711.2

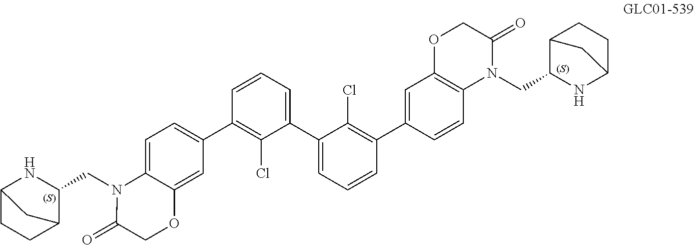

1H NMR (500 MHz, DMSO-d6) δ 9.14 (s, 2H), 8.87 (s, 2H), 7.52 (t, J=7.5 Hz, 2H), 7.47 (d, J=7.4 Hz, 2H), 7.43-7.38 (m, 4H), 7.19 (d, J=8.3 Hz, 2H), 7.15 (s, 2H), 4.77 (dd, J=31.2, 15.1 Hz, 4H), 4.39-4.30 (m, 2H), 4.03 (s, 2H), 3.91 (d, J=14.2 Hz, 2H), 3.48 (s, 2H), 1.95 (d, J=10.9 Hz, 2H), 1.73 (dd, J=18.0, 10.2 Hz, 2H), 1.62 (dd, J=18.2, 9.8 Hz, 6H), 1.33 (d, J=8.7 Hz, 2H), 1.23 (s, 2H).

LCMS (ESI): calculated for C₄₂H₄₀Cl₂N₄O₄ (free form); [M+H]+: 735.2, found: 735.2.

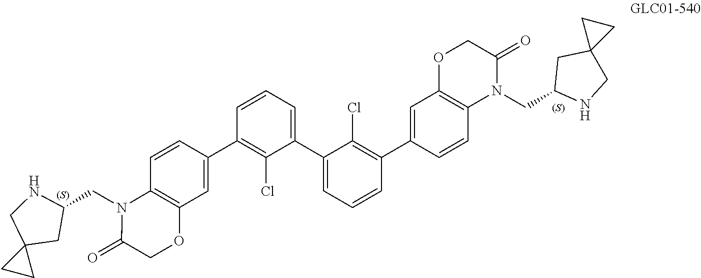

1H NMR (500 MHz, DMSO-d6) δ 9.82 (s, 2H), 9.03 (s, 2H), 7.53-7.46 (m, 6H), 7.41 (d, J=7.5 Hz, 2H), 7.17 (d, J=9.3 Hz, 2H), 7.14 (s, 2H), 4.78 (d, J=15.5 Hz, 4H), 4.41-4.30 (m, 4H), 3.94 (s, 2H), 3.22 (s, 2H), 3.11-3.05 (m, 2H), 2.02-1.92 (m, 4H), 0.74-0.61 (m, 8H).

LCMS (ESI): calculated for C₄₂H₄₀Cl₂N₄O₄ (free form); [M+H]+: 735.2, found: 735.2.

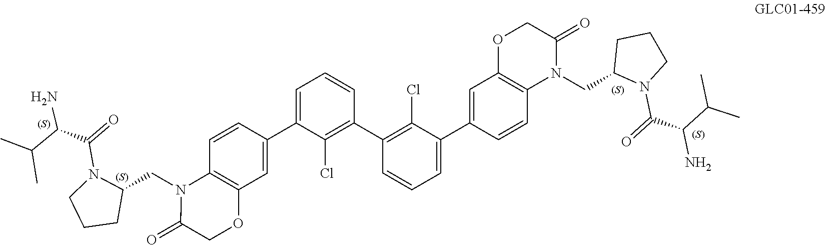

1H NIR (500 MHz, DMSO-d6) δ 8.21 (s, 6H), 7.74 (d, J=8.4 Hz, 2H), 7.54-7.45 (m, 4H), 7.40 (d, J=7.3 Hz, 2H), 7.16 (d, J=7.9 Hz, 2H), 7.11 (s, 2H), 4.72 (dd, J=39.8, 15.0 Hz, 4H), 4.45 (s, 2H), 4.27-4.19 (m, 2H), 3.95 (d, J=4.6 Hz, 2H), 3.80 (d, J=9.3 Hz, 2H), 3.68-3.56 (m, 4H), 2.11 (d, J=5.7 Hz, 4H), 1.97-1.89 (m, 2H), 1.83 (s, 4H), 1.00 (d, J=6.8 Hz, 6H), 0.91 (d, J=6.7 Hz, 6H).

LCMS (ESI): calculated for C₄₈H₅₄Cl₂N₆O₆ (free form); [M+H]⁺: 881.4, found: 881.4.

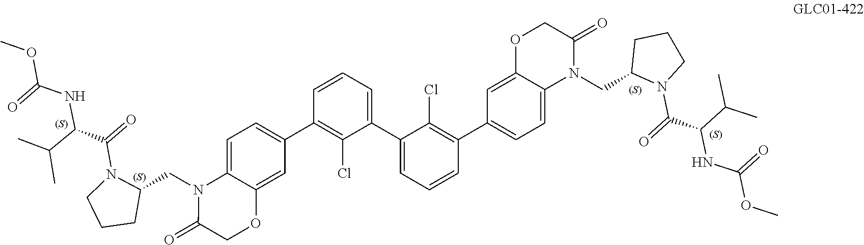

1H NMR (500 MHz, DMSO-d6) 7.73 (d, J=8.4 Hz, 2H), 7.53-7.44 (m, 4H), 7.39 (d, J=7.3 Hz, 2H), 7.15 (d, J=7.9 Hz, 2H), 7.09 (s, 2H), 4.72 (m, 4H), 4.44 (s, 2H), 4.27-4.19 (m, 2H), 3.95 (d, J=4.6 Hz, 2H), 3.80 (d, J=9.3 Hz, 2H), 3.70 (s, 6H), 3.68-3.56 (m, 4H), 2.11 (d, J=5.7 Hz, 4H), 1.97-1.89 (m, 2H), 1.83 (s, 4H), 1.00 (d, J=6.8 Hz, 6H), 0.91 (d, J=6.7 Hz, 6H).

LCMS (ESI): calculated for C₅₂H₅₈Cl₂N₆O₁₀; [M+H]+: 997.4, found: 997.4.

1H NMR (500 MHz, DMSO-d6) δ 9.74 (s, 2H), 8.84 (s, 2H), 7.53-7.44 (m, 6H), 7.41 (d, J=7.2 Hz, 2H), 7.18 (d, J=8.5 Hz, 2H), 7.14 (s, 2H), 5.32 (s, 2H), 4.76 (t, J=10.6 Hz, 4H), 4.35 (ddd, J=20.6, 17.7, 8.9 Hz, 8H), 3.91 (d, J=5.5 Hz, 2H), 3.00-2.93 (m, 2H), 2.09 (dd, J=12.9, 5.0 Hz, 2H), 1.87 (td, J=12.8, 4.7 Hz, 2H).

LCMS (ESI): calculated for C₃₈H₃₆Cl₂N₄O₆ (free form); [M+H]+: 715.2, found: 715.2.

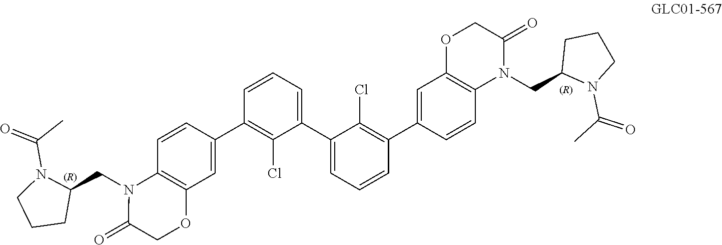

1H NMR (500 MHz, DMSO-d6) δ 7.78 (d, J=8.4 Hz, 2H), 7.53-7.44 (m, 4H), 7.40 (d, J=7.3 Hz, 2H), 7.17 (d, J=8.2 Hz, 2H), 7.10 (s, 2H), 4.78-4.64 (m, 4H), 4.24 (dd, J=24.9, 11.7 Hz, 4H), 3.74 (d, J=10.6 Hz, 2H), 3.52 (t, J=9.0 Hz, 2H), 3.38 (dd, J=17.3, 9.7 Hz, 2H), 2.09-2.01 (m, 2H), 1.94 (s, 6H), 1.92-1.87 (m, 2H), 1.80-1.70 (m, 4H).

LCMS (ESI): calculated for C₄₂H₄₀Cl₂N₄O₆; [M+H]+: 767.2, found: 767.2.

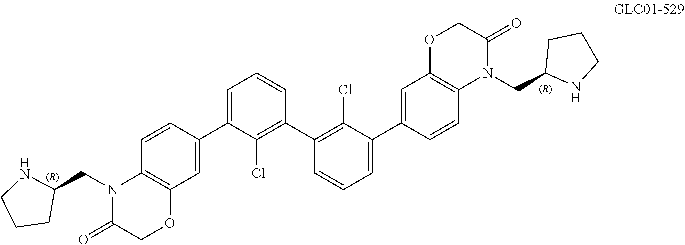

1H NMR (500 MHz, DMSO-d6) δ 9.65 (br s, 1H), 8.76 (br s, 1H), 7.54-7.49 (m, J=7.6 Hz, 1H), 7.46 (dd, J=9.0, 3.1 Hz, 2H), 7.41 (d, J=7.3 Hz, 1H), 7.16 (dd, J=8.2, 2.1 Hz, 1H), 7.13 (d, J=2.0 Hz, 1H), 4.77 (s, 2H), 4.38-4.25 (m, 2H), 3.70 (s, 1H), 3.31-3.04 (m, 2H), 2.20-1.95 (m, 2H), 1.94-1.66 (m, 2H).

LCMS (ESI): calculated for C₃₈H₃₆Cl₂N₄O₄; [M+H]+: 683.22, found: 683.49.

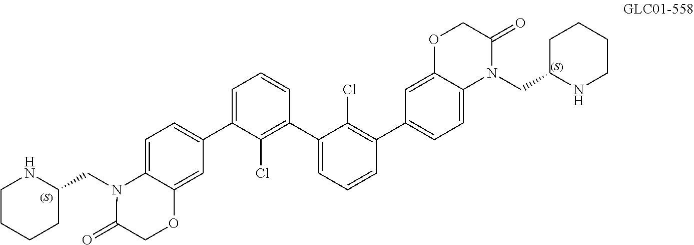

1H NMR (500 MHz, DMSO-d6) δ 9.07 (br s, 1H), 8.81 (br s, 1H), 7.52 (m, 1H), 7.48-7.44 (m, 2H), 7.41 (dd, J=7.5, 1.8 Hz, 1H), 7.18-7.10 (m, 2H), 4.77 (s, 2H), 4.35-4.01 (m, 2H), 3.30-3.31 (m, 2H), 2.84 (s, 1H), 2.02-1.91 (m, 2H), 1.73-1.61 (m, 2H), 1.56-1.42 (m, 2H).

LCMS (ESI): calculated for C₄₀H₄₀Cl₂N₄O₄; [M+H]+: 711.25, found: 711.43.

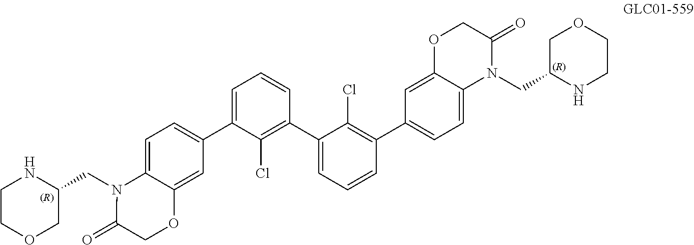

1H NMR (500 MHz, DMSO-d6) δ 9.74 (s, 1H), 9.40 (s, 1H), 7.51 (d, J=7.9 Hz, 2H), 7.47 (d, J=1.8 Hz, 1H), 7.41 (d, J=7.5 Hz, 1H), 7.15 (d, J=8.4 Hz, 1H), 7.12 (d, J=1.9 Hz, 1H), 4.77 (d, J=2.2 Hz, 2H), 4.36-4.25 (m, 1H), 4.16-4.04 (m, 2H), 3.91-3.74 (m, 2H), 3.69-3.53 (m, 2H), 3.32-3.26 (m, 2H).

LCMS (ESI): calculated for C₃₈H₃₆Cl₂N₄O₆; [M+H]+: 715.21, found: 715.39.

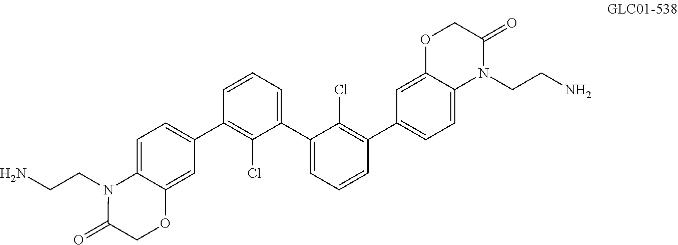 [SMILES: NCCN1C(=O)COc2cc(-c3cccc(-c4cccc(-c5ccc6c(c5)OCC(=O)N6CCN)c4Cl)c3Cl)ccc21]

1H NMR (400 MHz, DMSO-d6) δ 7.94 (s, 6H), 7.52 (t, J=6 Hz, 2H), 7.45 (d, J=4.0 Hz, 2H), 7.40 (d, J=4 Hz, 2H), 7.35 (d, J=4 Hz, 2H), 7.17 (d, J=4.0 Hz, 2H), 7.13 (s, 2H), 4.73 (s, 4H), 4.19 (t, J=6 Hz, 4H), 3.09 (t, J=6 Hz, 4H).

LCMS (ESI): calculated for C₃₂H₂₈Cl₂N₄O₄; [M+H]+: 603.15, found: 603.15.

### Example 12 (Asymmetric Asymmetric)

### Preparation of (S)-7-(2,2′-dichloro-3′-(3-oxo-3,4-dihydro-2H-benzo[b][1,4]oxazin-7-yl)-[1,1′-biphenyl]-3-yl)-4-((5-oxopyrrolidin-2-yl)methyl)-2H-benzo[b][1,4]oxazin-3(4H)-one

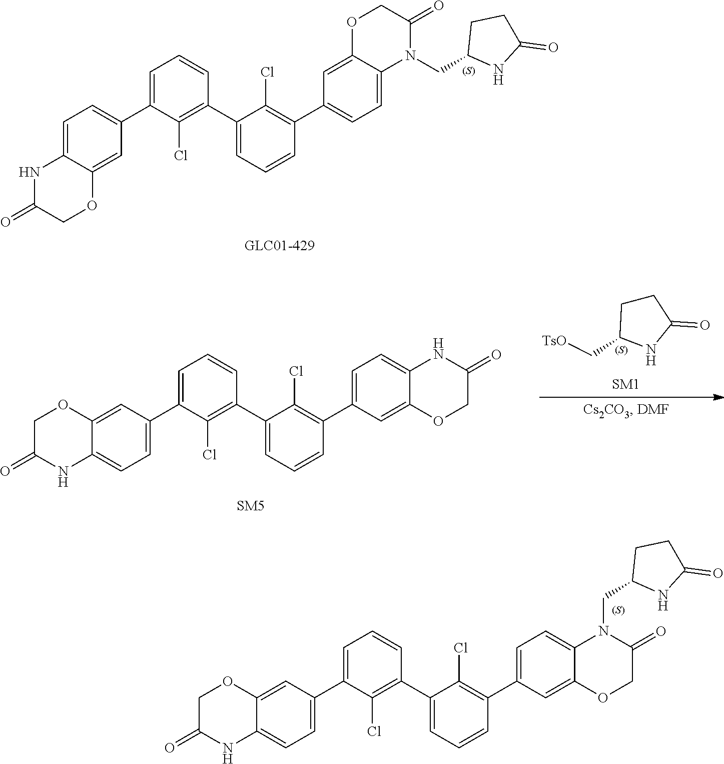

SM5 (20.0 mg 1 eq.), SM1 (10.4 mg, 1 eq.) and cesium carbonate (25 mg, 2 eq.) were dissolved in DMF (1 ml). The reaction was carried out at 40° C. for 2 h. After cooling, 5 ml water and 5 ml EA were added for extraction, and the organic phase was washed with water and concentrated to dryness. The residue was purified by column (DCM/MeOH=20/1) to give the title Compound 429 (11 mg, yield: 46.3%). ¹H NMR (500 MHz, DMSO-d₆) δ 10.82 (s, 1H), 7.76 (s, 1H), 7.52-7.46 (m, 2H), 7.46-7.41 (m, 2H), 7.41-7.36 (m, 2H), 7.34 (d, J=8.4 Hz, 1H), 7.14 (dd, J=8.3, 2.1 Hz, 1H), 7.10 (d, J=2.0 Hz, 1H), 7.07-7.02 (m, 2H), 6.98 (d, J=7.9 Hz, 1H), 4.71 (s, 2H), 4.63 (s, 2H), 4.04-3.92 (m, 3H), 2.34-2.23 (m, 1H), 2.21-2.05 (m, 2H), 1.83-1.74 (m, 1H). LCMS (ESI): calculated for C₃₃H₂₅Cl₂N₃O₅; [M+H]⁺: 614.13, found: 614.41.

Following the procedure above, the compound titled 385 was obtained by same procedure.

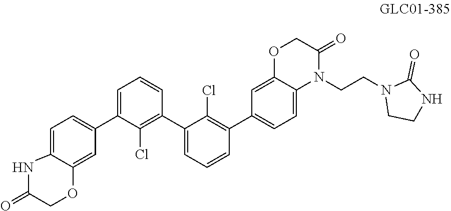 [SMILES: O=C1COc2cc(-c3cccc(-c4cccc(-c5ccc6c(c5)OCC(=O)N6CCN5CCNC5=O)c4Cl)c3Cl)ccc2N1]

¹H NMR (500 MHz, DMSO-d₆) δ 10.82 (s, 1H), 7.52-7.45 (m, 3H), 7.43 (dd, J=7.7, 1.8 Hz, 1H), 7.40-7.36 (m, 3H), 7.17 (dd, J=8.3, 2.0 Hz, 1H), 7.10 (d, J=2.0 Hz, 1H), 7.07-7.02 (m, 2H), 6.98 (d, J=7.9 Hz, 1H), 4.68 (s, 2H), 4.62 (s, 2H), 4.05 (t, J=6.5 Hz, 2H), 3.46-3.41 (m, 2H), 3.31 (t, J=6.5 Hz, 2H), 3.19-3.14 (m, 2H). LCMS (ESI): calculated for C₃₃H₂₆Cl₂N₄O₅; [M+H]⁺: 629.14, found: 629.44.

### Example 13

### 13A. Preparation of (2-(7-(2,2′-dichloro-3′-(3-oxo-4-(((S)-pyrrolidin-2-yl)methyl)-3,4-dihydro-2H-benzo[b][1,4]oxazin-7-yl)-[1,1′-biphenyl]-3-yl)-3-oxo-2,3-dihydro-4H-benzo[b][1,4]oxazin-4-yl)ethyl)-L-serine

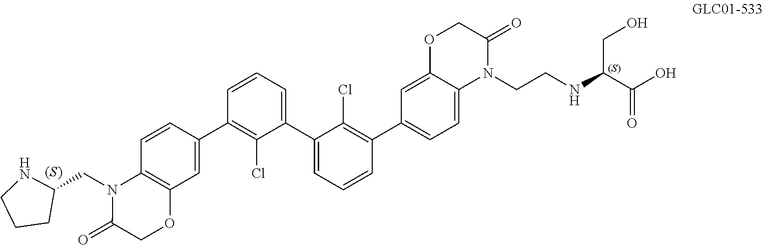

(a) Referring to the following reaction equation, Compound 1A-1 (0.35 g, 1.54 mmol, 1 eq.), Compound 1A-2 (1.10 g, 3.07 mmol, 2.0 eq.) and cesium carbonate (0.75 g, 2.30 mmol, 1.5 eq.) were dissolved in DMF (10 ml). The reaction was carried out at 60° C. for 5 h. After cooling, 10 ml water and 10 ml EA (ethyl acetate) were added for extraction, and the organic phase was washed with water and purified on silica gel, eluting with PE/EA (v/v, 8:1) to give Compound 1A, tert-butyl (S)-2-((7-bromo-3-oxo-2,3-dihydro-4H-benzo[b][1,4]oxazin-4-yl)methyl)pyrrolidine-1-carboxylate. (0.35 g, yield: 55.5%)

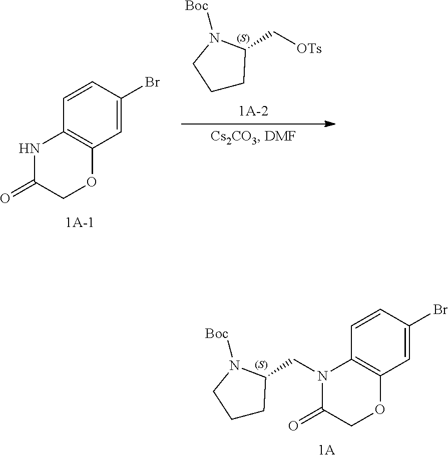

(b) Referring to the following reaction equation, compound 1B (25 mg) was dissolved in Dioxane (0.5 ml), then 1 N HCl solution (0.5 ml) was added dropwise, the temperature was raised to 85° C., and the reaction was carried out for 0.5 h. The pH of the reaction solution was adjusted to 7 to 8 by adding Na₂CO₃ saturated solution, and 10 ml EA was added for extraction. The organic phase was concentrated to give 1B (25 mg, yield: 107.0%).

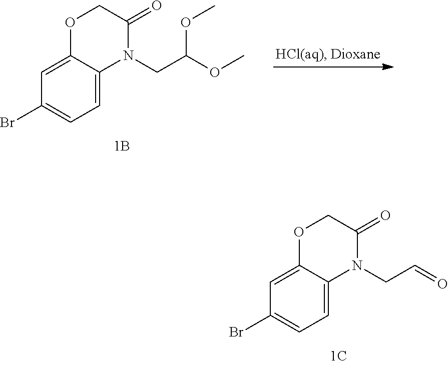 [SMILES: COC(CN1C(=O)COc2cc(Br)ccc21)OC ; O=CCN1C(=O)COc2cc(Br)ccc21]

(c) Referring to the following reaction equation, compound 1C (25 mg, 0.09 mmol, 1 eq.), 1D (29 mg, 0.19 mmol, 2 eq.), TEA (19 mg, 0.19 mmol) and one drop of AcOH was dissolved in DCM (3 ml), and the resulting mixture was allowed to stir at RT for 1 h. Then, NaBH(OAc)₃ (37 mg, 0.45 mmol, 5 eq.) was added, and the reaction was carried out for 1 h. Then, 10 ml H₂O and 10 ml DCM was added for extraction. The organic phase was concentrated to give 1C (25 mg, yield: 72%).

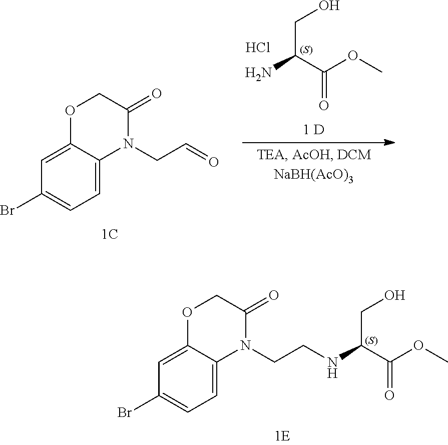

(d) Referring to the following reaction equation, Compound 1F (0.05 g, 0.11 mmol, 1 eq.), 1A (0.043 g, 0.11 mmol, 1 eq.), 1E (0.04 g, 0.11 mmol, 1 eq.), Pd(dppf)Cl2 (0.008 g, 0.01 mmol, 0.1 eq.) and potassium carbonate (0.058 g, 0.42 mmol, 4 eq.) were dissolved in Dioxane/H₂O (3 ml, v/v=5:1). The reaction was carried out at 85° C. for 2 h under N₂ atmosphere. After cooling, 10 ml water and 10 ml EA were added for extraction, and the organic phase was concentrated and purified by Prep-TLC (PE/EA=1/1 elution) to give Compound 1G, tert-butyl (S)-2-((7-(2,2′-dichloro-3′-(4-(2-(((S)-3-hydroxy-1-methoxy-1-oxopropan-2-yl)amino)ethyl)-3-oxo-3,4-dihydro-2H-benzo[b][1,4]oxazin-7-yl)-[1,1′-biphenyl]-3-yl)-3-oxo-2,3-dihydro-4H-benzo[b][1,4]oxazin-4-yl)methyl)pyrrolidine-1-carboxylate, 0.017 g, yield: 20%.

LCMS (ESI): calculated for C₄₄H₄₆Cl₂N₄O₉; [M+H]+: 845.26, found: 845.26.

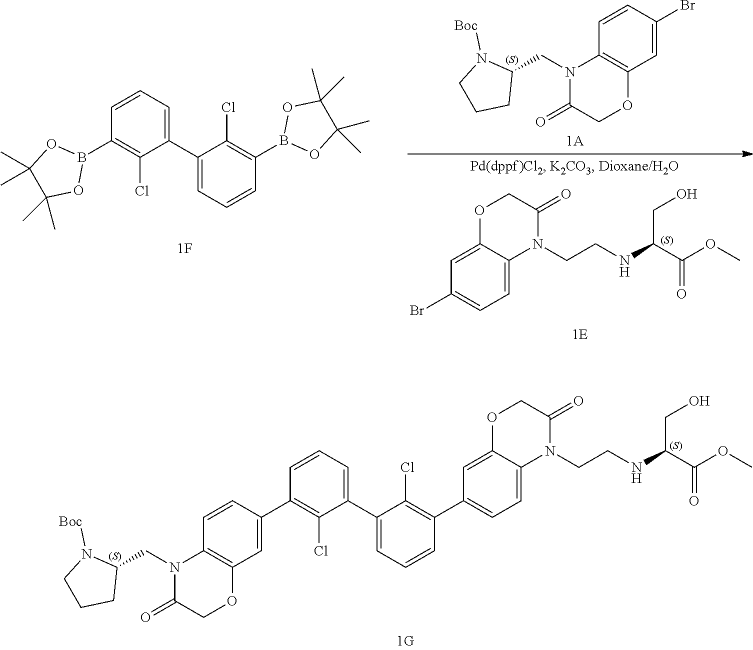

(e) Referring to the following reaction equation, 1N HCl (1 mL) was added to a solution of compound 1G (17 mg, 0.02 mmol) in Dioxane (1 mL) followed by stirring at rt for 3 h. Then, Na₂CO₃(aq) was added to the reaction mixture to neutralize the unreacted HCl. The reaction mixture was extracted with EA and water. The organic phase was concentrated and purified by Prep-HPLC to give 1F (10 mg, yield: 67%).

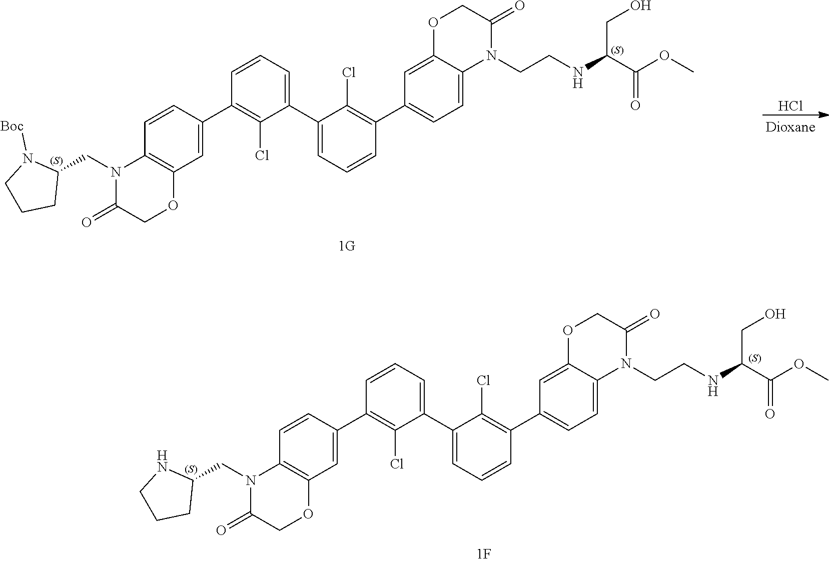

(f) Referring to the following reaction equation, NaOH (0.5 ml, 0.26M in water) was added to a solution of 1F (10 mg, 0.013 mmol) in MeOH/THF (3 mL, 2:1) followed by stirring at rt for 1 h. Then, HCl (1M) was added to the reaction mixture to neutralize the unreacted NaOH. The reaction mixture was purified by Prep-HPLC to give the title compound 1. (7.4 mg, yield: 75.5%).

¹H NMR (400 MHz, DMSO-d₆) δ 9.08 (s, 1H), 8.43 (s, 1H), 7.53-7.36 (m, 8H), 7.19-7.12 (m, 4H), 5.58 (s, 1H), 4.78 (s, 2H), 4.71 (s, 2H), 4.36 (m, 1H), 4.32-4.13 (m, 3H), 4.04 (m, 1H), 3.87 (s, 2H), 3.72 (m, 1H), 3.21 (m, 2H), 3.12 (m, 2H), 2.20 (m, 1H), 1.94 (m, 2H), 1.70 (m, 1H).

LCMS (ESI): calculated for C₄₀H₃₆Cl₂N₄O₈; [M+H]⁺: 731.21, found: 731.21.

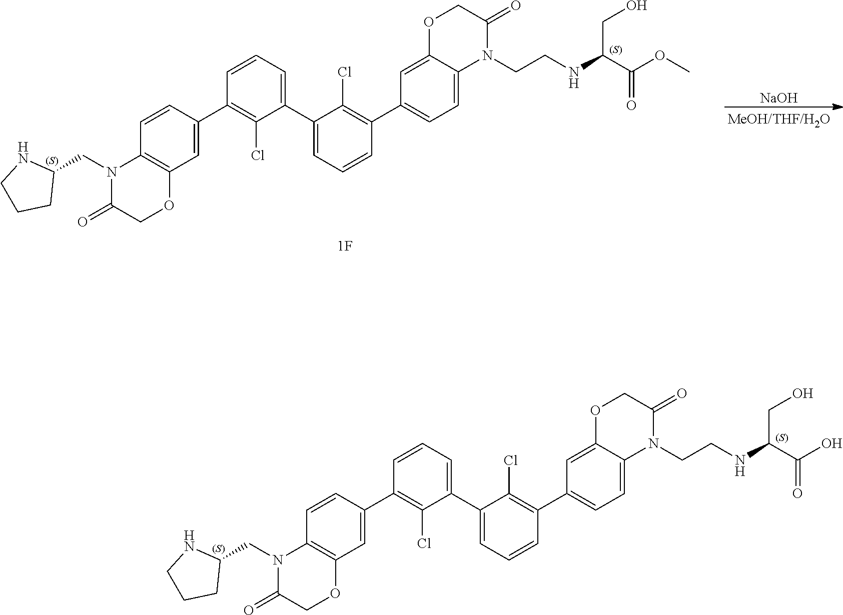

### 13B. The Following Compounds were Prepared Using the Same Procedure

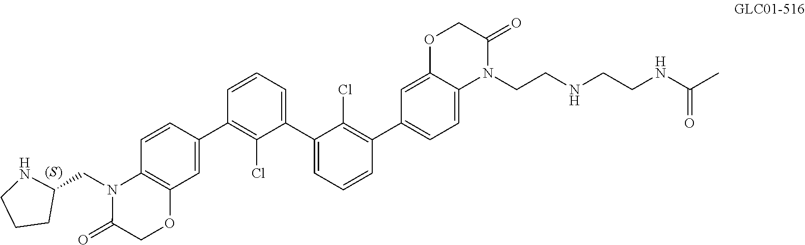

¹H NMR (400 MHz, DMSO-d₆) δ 9.09 (s, 1H), 8.64 (s, 2H), 8.44 (s, 1H), 8.15 (t, J=6.0 Hz, 1H), 7.52 (t, J=8.0 Hz, 2H), 7.45 (d, J=4.0 Hz, 2H), 7.41 (d, J=8.0 Hz, 2H), 7.36 (m, 2H), 7.17 (d, J=8.0 Hz, 2H), 7.14 (s, 2H), 4.75 (s, 2H), 4.71 (s, 2H), 4.36 (m, 1H), 4.24 (t, J=4.0 Hz, 2H), 419 (dd, J=12.0 Hz, 4.0 Hz, 1H), 3.72 (m, 1H), 3.31 (m, 3H), 3.23 (m, 2H), 3.12 (m, 1H), 3.06 (m, 2H), 2.20 (m, 1H), 1.97 (m, 2H), 1.85 (s, 3H), 1.70 (m, 1H)

LCMS (ESI): calculated for C₃₉H₃₉Cl₂N₅O₅; [M+H]⁺: 728.23, found: 728.23.

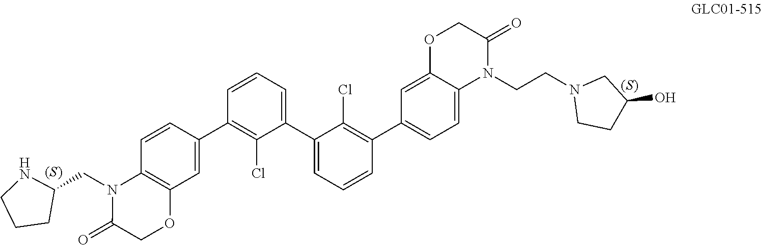

¹H NMR (400 MHz, DMSO-d₆) δ 9.09 (s, 1H), 8.44 (s, 1H), 7.53-7.49 (m, 2H), 7.47-7.43 (m, 2H), 7.42-7.33 (m, 4H), 7.21-7.12 (m, 3H), 7.10 (s, 1H), 5.54, (s, 1H), 4.75 (s, 2H), 4.71 (s, 2H), 4.46-4.40 (m, 2H), 4.34-4.28 (m, 2H), 4.06-3.89 (m, 3H), 3.78-3.66 (m, 2H), 3.44-3.40 (m, 2H), 3.27-3.03 (m, 2H), 2.32-2.20 (m, 2H), 2.18-2.08 (m, 2H), 2.02-1.91 (m, 2H), 1.75-1.86 (m, 2H).

LCMS (ESI): calculated for C₃₉H₃₈Cl₂N₄O₅; [M+H]⁺: 713.22, found: 713.22.

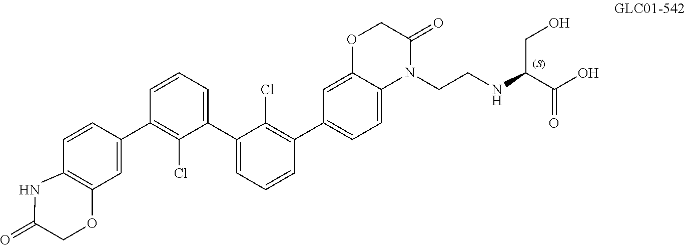

¹H NMR (400 MHz, DMSO-d₆) δ 10.84 (s, 1H), 7.51-7.37 (m, 7H), 7.17-6.97 (m, 5H), 4.71 (s, 2H), 4.62 (s, 2H), 4.15 (t, J=4.0 Hz, 2H), 3.70-3.62 (m, 3H), 3.05-2.99 (m, 2H).

LCMS (ESI): calculated for C₃₃H₂₇Cl₂N₃O₇; [M+H]⁺: 648.12, found: 648.12.

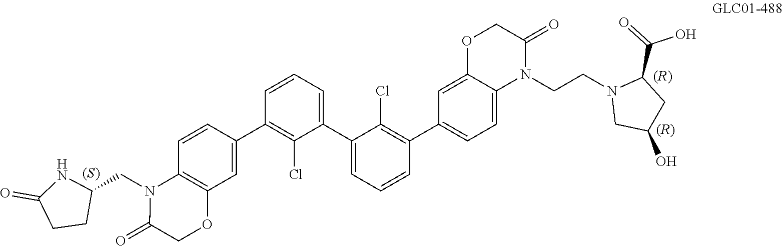

¹H NMR (400 MHz, DMSO-d₆) δ 8.01 (s, 1H), 7.53-7.48 (m, 2H), 7.47-7.44 (m, 2H), 7.41-7.38 (m, 2H), 7.36-7.33 (m, 2H), 7.18 (d, J=4.0 Hz, 1H), 7.15-7.12 (m, 2H), 7.10 (s, 1H), 4.75 (s, 2H), 4.71 (s, 2H), 4.22 (t, J=4.0 Hz, 2H), 4.05-4.00 (m, 1H), 3.96-3.93 (m, 2H), 3.69-3.58 (m, 1H), 3.29 (t, J=4.0 Hz, 2H), 3.27-3.25 (m, 3H), 2.16-2.06 (m, 2H), 2.30-2.28 (m, 2H), 2.12-2.02 (m, 2H).

LCMS (ESI): calculated for C₄₀H₃₆Cl₂N₄O₈; [M+H]⁺: 771.19, found: 771.19.

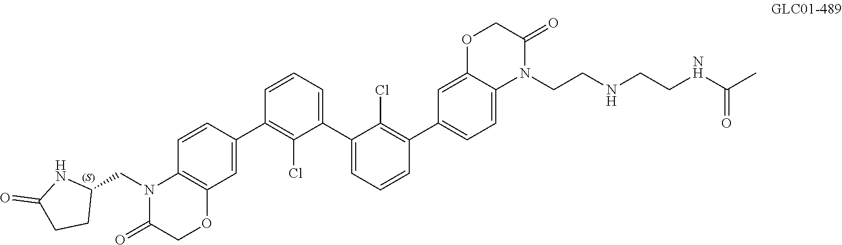

¹H NMR (400 MHz, DMSO-d₆) δ 8.61 (s, 2H), 8.14 (t, J=4.0 Hz, 1H), 7.78 (s, 1H), 7.53-7.48 (m, 2H), 7.47-7.44 (m, 2H), 7.41-7.38 (m, 2H), 7.36-7.33 (m, 2H), 7.18 (d, J=4.0 Hz, 1H), 7.15-7.12 (m, 2H), 7.10 (s, 1H), 4.75 (s, 2H), 4.71 (s, 2H), 4.24 (t, J=4.0 Hz, 2H), 4.04-4.00 (m, 1H), 3.97-3.92 (m, 2H), 3.32-3.30 (t, J=4.0 Hz, 2H), 3.25-3.21 (m, 2H), 3.08-3.04 (m, 2H), 2.31-2.26 (m, 2H), 2.17-2.07 (m, 2H), 1.85 (s, 3H).

LCMS (ESI): calculated for C₃₉H₃₇Cl₂N₅O₆; [M+H]⁺: 742.21, found: 742.21.

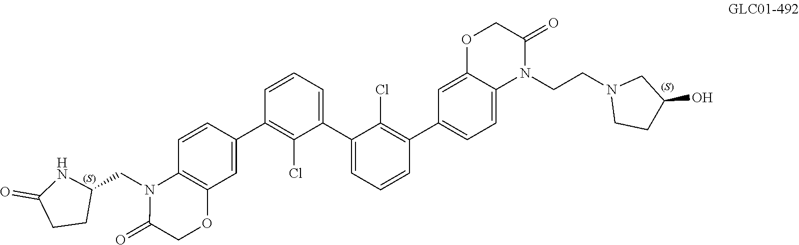

¹H NMR (400 MHz, DMSO-d₆) δ 7.78 (s, 1H), 7.53-7.49 (m, 2H), 7.47-7.43 (m, 2H), 7.42-7.33 (m, 4H), 7.21-7.12 (m, 3H), 7.10 (s, 1H), 5.54, (s, 1H), 4.75 (s, 2H), 4.71 (s, 2H), 4.46-4.40 (m, 2H), 4.34-4.28 (m, 2H), 3.89-4.06 (m, 3H), 3.78-3.66 (m, 2H), 3.27-3.03 (m, 2H), 2.32-2.20 (m, 2H), 2.18-2.08 (m, 2H), 2.02-1.91 (m, 2H), 1.75-1.86 (m, 2H).

LCMS (ESI): calculated for C₃₉H₃₆Cl₂N₄O₆; [M+H]⁺: 727.20, found: 727.20.

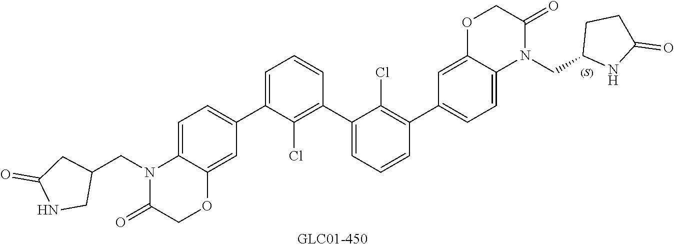

¹H NMR (500 MHz, DMSO-d₆) δ 7.76 (br s, 1H), 7.55 (br s, 1H), 7.53-7.47 (m, 2H), 7.48-7.44 (m, 2H), 7.42-7.36 (m, 3H), 7.34 (d, J=8.4 Hz, 1H), 7.18-7.13 (m, 2H), 7.11 (dd, J=6.9, 2.0 Hz, 2H), 4.73 (s, 2H), 4.71 (s, 2H), 4.09-3.90 (m, 5H), 3.34-3.29 (m, 1H), 3.06-3.01 (m, 1H), 2.82-2.75 (m, 1H), 2.30-2.23 (m, 2H), 2.18-2.05 (m, 2H), 2.02-1.96 (m, 1H), 1.83-1.74 (m, 1H). LCMS (ESI): calculated for C₃₈H₃₂Cl₂N₄O₆; [M+H]⁺: 711.18, found: 711.51.

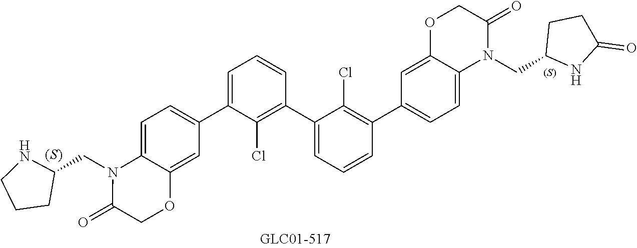

¹H NMR (500 MHz, DMSO-d₆) δ 7.76 (br s, 1H), 7.53-7.44 (m, 4H), 7.42-7.32 (m, 4H), 7.18-7.10 (m, 4H), 4.74 (s, 2H), 4.71 (s, 2H), 4.38-4.25 (m, 2H), 4.04-3.70 (m, 4H), 3.31-3.04 (m, 2H), 2.40-1.66 (m, 8H).

LCMS (ESI): calculated for C₃₈H₃₄Cl₂N₄O₅; [M+H]⁺: 697.20, found: 697.47.

### Example 14

### PD-1/PD-L1 Homogeneous Time-Resolved Fluorescence (HTRF) Binding Assay

The assays were conducted in a standard black 384-well polystyrene plate with a final volume of 20 μL. Inhibitors were first serially diluted in DMSO and then added to the plate wells before the addition of other reaction components. The final concentration of DMSO in the assay was 1%. The assays were carried out at 25° C. in the PBS buffer (pH 7.4) with 0.05% Tween-20 and 0.1% BSA. Recombinant human PD-L1 protein (19-238) with a Histag at the C-terminus was purchased from AcroBiosystems (PD1-H5229). Recombinant human PD-1 protein (25-167) with Fe tag at the C-terminus was also purchased from AcroBiosystems (PD1-H5257). PD-L1 and PD-1 proteins were diluted in the assay buffer and 10 μL was added to the plate well. Plates were centrifuged and proteins were preincubated with inhibitors for 40 minutes. The incubation was followed by the addition of 10 μL of HTRF detection buffer supplemented with Europium cryptate-labeled anti-human IgG (PerkinElmer-AD0212) specific for Fe and anti-His antibody conjugated to SureLight®-Allophycocyanin (APC, PerkinElmer-AD0059H). After centrifugation, the plate was incubated at 25° C. for 60 min. before reading on a PHERAstar FS plate reader (665 nm/620 nm ratio). Final concentrations in the assay were—3 nM PD1, 10 nM PD-L1, 1 nM europium anti-human IgG and 20 nM anti-His-Allophycocyanin. IC₅₀ determination was performed by fitting the curve of percent control activity versus the log of the inhibitor concentration.

TABLE 1

| Com- | | Com- | | Com- | |
|---|---|---|---|---|---|
| pound | HTRF | pound | HTRF | pound | HTRF |
| ID | IC₅₀ (nm) | ID | IC₅₀ (nm) | ID | IC₅₀ (nm) |
| 281 | 26.74 | 454 | 1.42 | 523 | 10.89 |
| 303 | 187.43 | 457 | 8.78 | 527 | 9.41 |
| 338 | 1.46 | 458 | 1686.1 | 528 | 5.04 |
| 448 | 5.45 | 459 | 435.78 | 529 | 8.62 |
| 354 | 2184.69 | 460 | 9.9 | 530 | 76.88 |
| 361 | 1.75 | 461 | 4.06 | 531 | 7.22 |
| 363 | 2.16 | 465 | 3.58 | 533 | 7.11 |
| 372 | 3000 | 466 | 5 | 535 | 4.41 |
| 373 | 3.79 | 468 | 4.9 | 536 | 4.31 |
| 374 | 68.16 | 469 | 4.02 | 538 | 8.18 |
| 375 | 2707.11 | 470 | 2.28 | 539 | 7.73 |
| 376 | 199.54 | 471 | 4.7 | 540 | 10.5 |
| 378 | 69.66 | 472 | 5.27 | 542 | 5.24 |
| 381 | 145.86 | 475 | 3.23 | 545 | 4.81 |
| 382 | 3000 | 476 | 5.22 | 550 | 3.96 |
| 384 | 260.37 | 478 | 8.76 | 551 | 2.64 |
| 385 | 263.89 | 479 | 6.02 | 552 | 7.25 |
| 386 | 3000 | 480 | 5.48 | 554 | 5.51 |
| 388 | 1.1 | 481 | 4.69 | 557 | 5.33 |
| 394 | 470.58 | 482 | 6.72 | 558 | 10.64 |
| 395 | 6.26 | 483 | 6.31 | 559 | 8.98 |
| 396 | 11.66 | 484 | 6.53 | 560 | 7.78 |
| 397 | 304.73 | 486 | 1382.63 | 561 | 8.95 |
| 401 | 9.78 | 487 | 650.11 | 563 | 7.18 |
| 402 | 1.98 | 488 | 6.2 | 568 | 11.27 |
| 403 | 191.26 | 489 | 6.31 | 570 | 10.87 |
| 408 | 571.47 | 490 | 3.51 | 571 | 16.04 |
| 411 | 6.14 | 491 | 5.22 | 573 | 287.3 |
| 412 | 3000 | 492 | 3.15 | 576 | 10.94 |
| 413 | 7.05 | 493 | 5.59 | 585 | 10.87 |
| 414 | 737.11 | 494 | 6.95 | 588 | 7.35 |
| 415 | 103.69 | 500 | 10.69 | 590 | 6.04 |
| 422 | 3000 | 503 | 4.19 | 591 | 17.8 |
| 427 | 68.65 | 513 | 8.23 | 596 | 6.47 |
| 429 | 20.81 | 514 | 4.46 | 597 | 10.14 |
| 430 | 3000 | 515 | 8.71 | 607 | 13.89 |
| 445 | 5.01 | 516 | 10.5 | 608 | 28.1 |
| 449 | 3.89 | 517 | 7.46 | 614 | 7.7 |
| 450 | 6.47 | 518 | 7.19 | | |

### Example 15

### PD-L1 Internalization

### 1. Experiment Protocol

Day 1. Seeding cells: Digest the PD-L1/CHO-K1 cells by trypsin in the flask, then counting the cell number and diluted to 1×105 cells/ml. Seeding cells to 6 well plate (Corning, #3516), 2 mL/well. The plates were incubated at 37° C., 50% CO₂ incubator for 24 hours.

Day 2. Preparing compounds and treated cells: Dilute GLC01-258 from 15 mM to 0.5 mM by using DMSO, and 15 mM compound were serial diluted from 15 mM˜15 nM by DMSO, Then 500 times dilute the compounds by using assay buffer. Prepare 0.2% DMSO in assay buffer used for Vehicle control and low control. Take out the plates, aspirated the medium and thrown away. Adding 2 mL of the diluted compounds, vehicle control and low control to corresponding wells. Then incubated the plates for 17 hours at 37° C., 5% CO₂ incubator.

Day 3. Preparing samples for FACS: After 17 hours incubation, discarded medium and washed by the PBS. Cells in each well were Digested by trypsin. Centrifuge and discard the supernatant, then wash the cells by DPBS (Ca²⁺, Mg²⁺ free) for twice. 10 fold dilute the antibody (PE-conjugated mouse anti-human CD274) by DPBS, then adding the staining solution to compounds treated samples and vehicle control sample. The low control only add DPBS without antibody. Incubate the plates at room temperature for 20 minutes and protect from light. After 20 minutes, wash the samples by DPBS for twice. Then centrifuge and discard the supernatant. Resuspend cells by 300 uL DPBS and transfer the samples to 5 mL Polystryrene Round-Bottom Tube (Falcon, #352054), then tested by BD FACSCanto. Test the samples by BD FACSCanto.

### 2. Data Analysis

Set the PD-L1 signal of vehicle control as 100%, PD-L1 signal of Low control as 0%. Then calculate the compound treated samples' PD-L1 signal

0% PD-L1 signal: Low control which stained without anti-CD274

100% PD-L1 Signal: vehicle control which stained with anti-CD274

The % activation of PD-L1 Internalization=1−PD-L1 signal of compound.

TABLE 2

| Compound | |
|---|---|
| ID | Internalization |
| 338 | 6.80% |
| 363 | 5.50% |
| 373 | 3.20% |
| 388 | 6.10% |
| 395 | 2.50% |
| 402 | 2.40% |
| 411 | 66.90% |
| 445 | 20.50% |
| 449 | 12.20% |
| 454 | 5.20% |
| 460 | 1.90% |
| 465 | 67.00% |
| 468 | 20.90% |
| 470 | 20.90% |
| 472 | 17.20% |
| 475 | 31.50% |
| 476 | 6.10% |
| 480 | 5.70% |
| 481 | 8.70% |
| 483 | 19.80% |
| 500 | 19.50% |
| 503 | 3.50% |
| 517 | 22.80% |
| 523 | 73.90% |
| 528 | 13.30% |
| 529 | 98.10% |
| 530 | 1.60% |
| 533 | 32.70% |
| 535 | 21.00% |
| 536 | 7.90% |
| 538 | 3.60% |
| 539 | 11.60% |
| 540 | 10.60% |
| 542 | 3.30% |
| 545 | 7.80% |
| 550 | 3.00% |
| 551 | 3.10% |
| 552 | 4.90% |
| 553 | 9.40% |
| 554 | 23.20% |
| 557 | 20.30% |
| 560 | 3.10% |
| 561 | 5.30% |
| 563 | 25.50% |
| 585 | 39.00% |
| 588 | 2.00% |
| 590 | 3.50% |
| 596 | 2.90% |
| 608 | 2.60% |
| 613 | 4.80% |
| 614 | 45.70% |
| 620 | 43.90% |

### Example 16

### PD-L1 Dimerization

Compounds were tested in biochemical protein-protein interaction assays to determine if they can specifically dimerize the extracellular domains of PD-L1.
(1) Dilute cpd 1:3 in succession in DMSO for each column for 10±0 pts (refer to dilution plate map)
(2) Transfer 0.2 μL cpds solution in each row to 384 assay plate using Echo, each column containing 2 replicates (refer to assay plate map).
(3) Add 20 μL prepared mixture include PDL1-Eu and PDL1-A2 solution to assay plate, centrifuge at 1000 rpm for 1 min.
(4) Incubate at 25° C. for 120 min.
(5) Read fluorescence signal on Envision 2104 plate reader.
(6) Read the Ratio (665 nm/615 nm) signal on Envision.
(7) Analyze the raw data using the equation (V. Data analysis)

TABLE 3

| | Dimerization |
|---|---|
| Compound | EC₅₀ |
| ID | (nm) |
| 411 | 356.8 |
| 454 | 326.4 |
| 465 | 77.9 |
| 472 | 358.6 |
| 523 | 1330 |
| 528 | 174.9 |
| 533 | 90.96 |
| 535 | 354.3 |
| 550 | 224.7 |
| 554 | 55.82 |
| 563 | 37.71 |
| 585 | 130.3 |
| 588 | 154.4 |
| 590 | 220.1 |
| 596 | 121.3 |
| 614 | 50.36 |
| 620 | 61.6 |
| 641 | 58.11 |
| 648 | 59.15 |
| 649 | 63.7 |

### Example 17

### PDL1 Jurkat-NFAT Reporter Assay

a. Preparation of Hep3B-OS8-hPDL1
1. Hep3B-OS8-hPDL1 cells were cultured in 1640 medium supplemented with 10% fetal bovine serum, 1% penicillin and streptomycin, in which 100 μg/mL G418 and Hygromycin B were also added.
2. Cells were re-suspended with RPMI 1640 medium containing 10% FBS and the cell density was adjusted to 1.25E5 cells/mL.
3. Cells were seeded into 96-well flat bottom plate (1.25E4 cells/100 μL/well).
   b. Compounds Solutions Preparation
4. Remove the medium from pre-plated Hep3B-OS8-PDL1 cells. Wash once with 200 μL Assay Medium.
5. Prepare the Compounds dilutions in RPMI 1640 medium with 10% FBS according to the Layout.
6. Add compounds into each well in a volume of 50 μL in 9 concentrations (3, 1, 0.3, 0.1, 0.03, 0.01, 0.003, 0.001 and 0.0003 μM)}. Keytruda will be included as positive control at the concentration of 5 μg/mL.
7. Incubate for 20-30 min at 37° C., 5% CO₂.
   c. Preparation of Jurkat-NFAT-PD1
8. Jurkat-NFAT-PD1 cells were cultured in 1640 medium supplemented with 10% fetal bovine serum, 1% penicillin and streptomycin, in which 1000 μg/mL Hygromycin B and 0.3 μg/mL puromycin were also added.
9. On the second day of assay, the cells were re-suspended with RPMI 1640 medium containing 10% FBS and the cell density was adjusted to 2.5E5 cells/mL.
10. Cells were seeded into 96-well flat bottom plate (1.25E4 cells/50 μL/well).
11. Incubate the assay plate in a humidified 37° C., 5% CO₂ incubator for 6 hours.
12. Equilibrate cultured cells at room temperature for 5-10 mins.
13. Add equal volume (100 μL/well) of ONE-Glo™ Luciferase Assay System to each well, wait at least 3 mins to allow complete cell lysis and measure in a luminometer.

TABLE 4

| Compound | Jurkat-NFAT EC₅₀ |
|---|---|
| ID | (nm) |
| 465 | 0.3 |
| 529 | 111.1 |

### Example 18

a. Hep3B-OS8-hPDL1 and T Cells Co-Culture Assay

### Tumor Preparation

1. Hep3B-OS8-hPDL1 cells were cultured in 1640 medium supplemented with 10% fetal bovine serum, 1% penicillin and streptomycin, in which 100 μg/mL G418 and Hygromycin B were also added.
2. Hep3B-OS8-hPDL1 cells were harvested and treated with 10 μg/mL mitomycin C at 37° C. for 1.5 h, and the cells were then washed thoroughly with PBS for four times.
3. Cells were re-suspended with RPMI 1640 medium containing 10% FBS and the cell density was adjusted to 5E5 cells/mL.
4. Cells were seeded into 96-well flat bottom plate (2.5E4 cells/50 μL/well).
   b. CD3+ T Cells Isolation (30 mL Blood)
5. The human blood sample from an individual donor is diluted by the same volume of sterile PBS, for instance add 25 mL sterile PBS into 25 mL fresh whole blood and mix sufficiently by gentle shake.
6. Transfer 15 mL Lymphoprep medium into a new 50 mL centrifuge tube.
7. Add the diluted blood sample as soft as possible onto the surface of the Ficoll medium to make sure there is a clear dividing line between the two liquids, and the volume ratio between Ficoll and diluted blood (30 mL) is 1:2.
8. Move the tube gently to centrifuge, at 1000×g, 25 min, 20° C., with the acceleration (5) and Min deceleration (0) settings during the centrifugation.
9. Four interfaces in all could be observed after the centrifugation, which are layers of plasma, mononuclear cells, the Ficoll medium and RBCs from top to bottom, and move the tube as gently as possible so as keep the four interfaces separated. Absorb and transfer the second layer of mononuclear cells into another new sterile centrifuge tube carefully, and absorb certain volume of plasma instead of the Ficoll medium if inevitable.
10. Add sterile PBS of the three times the volume of PBMCs in the tube with PBMC.
11. Wash the cells twice with 5-10 mL PBS before cell counting with cytometer. Centrifuge at 350×g, 10 min, 20° C. With the acceleration (5) and deceleration (5) settings during the centrifugation.
12. Resuspend the cells with recommended medium and adjust the density of the PBMCs to the final concentration of 5E7 cells/mL.
13. Isolate CD3+ T cells with EasySep™ Human T Cell Isolation Kit (STEMCELL Technologies #17951), and the cells were seeded into 96-well flat bottom plate (5E4 cells/100 μL/well). c. Compounds solutions preparation
14. Prepare the Compounds dilutions in RPMI 1640 medium with 10% FBS according to the Layout. 15. Add compounds into each well in a volume of 50 μL. {3 compounds (GLC01-258, GLC01-269, GLC01-465) in 7 concentrations (0.03, 0.1, 0.3, 1, 3, 10 and 30 μM) and 6 compounds (GLC01-411, GLC01-292, GLC01-445, GLC01-475, GLC01-470 and GLC01-468) in the same concentration (1 μM)}.
16. Keytruda will be included as positive control, the concentration will be 5 μg/mL.
17. Incubate for 72 hours at 37° C., 5% CO2.
18. Collect supernatant by centrifugation and measure IFN-γ by ELISA.

TABLE 5

| Compound | Tumor and T Cell EC₅₀ |
|---|---|
| ID | (nm) |
| 465 | 1.2 |
| 529 | 324.9 |
| 533 | 6.032 |
| 554 | 5.1 |

### Example 19

### Mouse PK Study

(1) The compounds were weighed and dissolved in the vehicle of 1 mg/mL 5% solutol in saline, shaken well, and sonicated to form a colorless clear solution. The solution was given orally at a dose of 10 mg/kg to a group of 3 mice after an overnight fast.
(2) Blood was collected from the submandibular vein, and heparin sodium was used for anticoagulation. The blood was placed on ice after collection and plasma was separated by centrifugation within 1 hour (centrifugation conditions: 8000 rpm, 6 minutes, 2-8° C.). Blood sampling time points were 0.083, 0.25, 0.5, 1, 2, 4, 8, and 24 hours.
(3) The samples were stored in a −20° C. freezer. The plasma sample (40 μL) was added with 160 μL of ice-cold acetonitrile containing internal standard, vortexed for 3 minutes, and centrifuged at 11,000 rpm for 5 minutes. 100 μL of the supernatant was added to 100 μL of water, and 5 μL of the supernatant was injected in LC/MS/MS instrument to detect the compound (if the compound was an ester, acid was detected).

Data are in Table 6:

TABLE 6

| Compound | Observed | Cₘₐₓ | Tₘₐₓ | AUCₗₐₛₜ | T_{1/2} |
|---|---|---|---|---|---|
| ID | Compound | (ng/mL) | (h) | (h · ng/mL) | (h) |
| 527 | 528 | 312 | 0.333 | 326 | 1.04 |
| 537 | 465 | 1426 | 0.333 | 1280 | 1.11 |
| 541 | 465 | 1533 | 0.333 | 1367 | 1.04 |
| 566 | 465 | 311 | 0.500 | 520 | 2.48 |
| 567 | 529 | N.D. | N.D. | N.D. | N.D. |
| 589 | 554 | 575 | 0.250 | 338 | 1.04 |
| 594 | 465 | 916 | 0.500 | 1307 | 1.47 |
| 633 | 614 | 473 | 0.500 | 507 | N.D. |
| 634 | 639 | 702 | 0.500 | 571 | N.D. |
| 631 | 641 | 2160 | 0.667 | 2419 | N.D. |
| 638 | 629 | 4210 | 0.750 | 4639 | N.D. |
| 619 | 620 | 5237 | 0.500 | 10077 | N.D. |
| 640 | 647 | 3447 | 0.667 | 4055 | N.D. |
| 650 | 563 | 409 | 0.667 | 542 | N.D. |
| 652 | 653 | 68.2 | 0.833 | 94.7 | N.D. |

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the present application, and it is not intended to detail all those obvious modifications and variations of it which will become apparent to the skilled worker upon reading the description. It is intended, however, that all such obvious modifications and variations be included within the scope of the present application, which is defined by the following claims. The claims are intended to cover the components and steps in any sequence which is effective to meet the objectives there intended, unless the context specifically indicates the contrary.

1. A compound of formula (I): 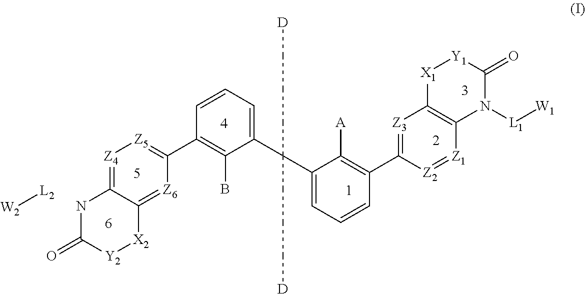
or a pharmaceutically acceptable salt, stereoisomer, mixture of stereoisomers, solvate, or tautomer thereof, wherein,
A and B each is independently selected from halogen, cyano, —N₃, alkyl and substituted alkyl, amine, alkylamine, alkoxy;
Z₁ is —CR¹═ or N═;
Z₂ is —CR²═;
Z₃ is —CR³═ or —N═;
Z₄ is —CR⁴═ or N═;
Z₅ is —CR⁵═;
Z₆ is —CR⁶═ or —N═;
R¹ and R⁴ each is independently —H, halogen, cyano, alkyl, cycloalkyl, substituted alkyl, alkenyl, alkynyl, aryl, amine, alkylamine, alkoxy;
R² and R⁵ each is independently —H, halogen, cyano, alkyl, cycloalkyl, substituted alkyl, alkenyl, alkynyl, aryl, amine, alkylamine, alkoxy;
R³ and R⁶ each is independently —H, halogen, cyano, alkyl, cycloalkyl, substituted alkyl, alkenyl, alkynyl, aryl, amine, alkylamine, alkoxy;
Y₁ and Y₂ each is independently —C(R⁷)(R⁸)—, —CR⁹—, —NR¹⁰—, —O—, or —S—;
X₁ and X₂ each is independently —C(R¹¹)(R¹²)—, —N═, —NR¹³—, —S— or —O—;
R⁷, R⁸, R⁹, R¹¹, and R¹² each is independently —H, halogen, cyano, alkyl, cycloalkyl, substituted alkyl, alkenyl, alkynyl, aryl, amine, alkylamine, alkoxy;
R¹⁰ and R¹³ each is independently —H, alkyl, cycloalkyl, substituted alkyl, alkenyl, alkynyl, aryl, alkylamine, alkoxy;
L₁ and L₂ each is an alkyl, substituted alkyl or hetereoatom chain, containing m atoms between Ring 3 and W₁, and Ring 6 and W₂, wherein m=0, 1, 2, 3, 4, 5 or 6; when m is 0, W₁ or W₂ is directly linked to the corresponding nitrogen in ring 3 or ring 6, respectively;
W₁ and W₂ each is independently hydrogen, a five member heterocyclic ring or substituted five member heterocyclic ring, a six member heterocyclic ring or substituted six member heterocyclic ring, a carboxylalkyl group or substituted carboxylalkyl group, a cyanoalkyl group or substituted cyanoalkyl group, an aminoalkyl group or substituted aminoalkyl group, a hydroxyalkyl group or substituted hydroxyalkyl group, an amino acid, an ester of amino acid, an amide of amino acid, a unnatural amino acid, an ester of unnatural amino acid or an amide of unnatural amino acid.

2. The compounds of claim 1, wherein
A and B each is independently selected from halogen, alkyl and substituted alkyl, cyano, and —N₃,
Z₁ is —CR¹═ or —N═;
Z₂ is —CR²;
Z₃ is —CR³═ or —N═;
Z₄ is —CR⁴ or —N═,
Z₅ is —CR⁵═;
Z₆ is —CR⁶═ or —N═;
R¹ and R⁴ each is independently —H or —F, —Cl, or —CH₃;
R² and R⁵ each is independently, —H, —Cl, —F, —CH₃, or —NH₂;
R³ and R⁶ each is independently —H, —Cl, —F, —CH₃, or —NH₂;
X₁ and X₂ each is independently —C(R¹¹)(R¹²)—, —N═, —NH—, —N(R¹³)— or —O—;
Y₁ and Y₂ each is independently —CH₂—, —CH═, —NH—, —O—, —C(R⁷)(R⁸)—;
R⁷, R⁸, R¹¹, and R¹² each is independently —H, —F, —Cl, or —CH₃;
L₁ and L₂ each is an alkyl or substituted alkyl containing m carbon atoms, wherein m=0, 1, 2, 3, 4, 5 or 6; when m is 0, W₁ or W₂ is directly linked to the corresponding nitrogen in ring 3 or ring 6, respectively;
W₁ and W₂ each is independently hydrogen, a five member heterocyclic ring or substituted five member heterocyclic ring, a six member heterocyclic ring or substituted six member heterocyclic ring, a carboxylalkyl group or substituted carboxylalkyl group, a cyanoalkyl group or substituted cyanoalkyl group, an aminoalkyl group or substituted aminoalkyl group, a hydroxyalkyl group or substituted hydroxyalkyl group, an amino acid, an ester of amino acid, an amide of amino acid, a unnatural amino acid, an ester of unnatural amino acid or an amide of unnatural amino acid.

3. The compound of claim 1, wherein the compound comprises a core structure selected from the group consisting of formulas (II) to (XXIII): 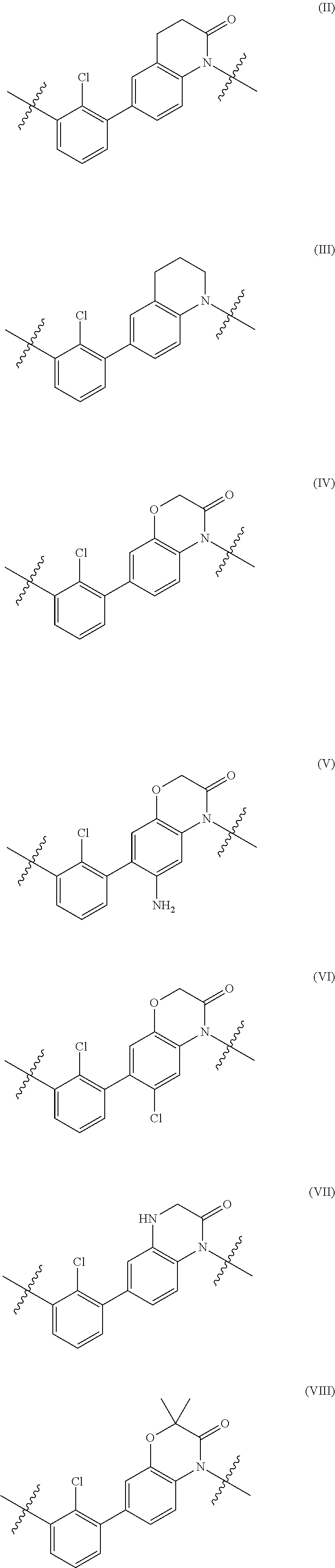 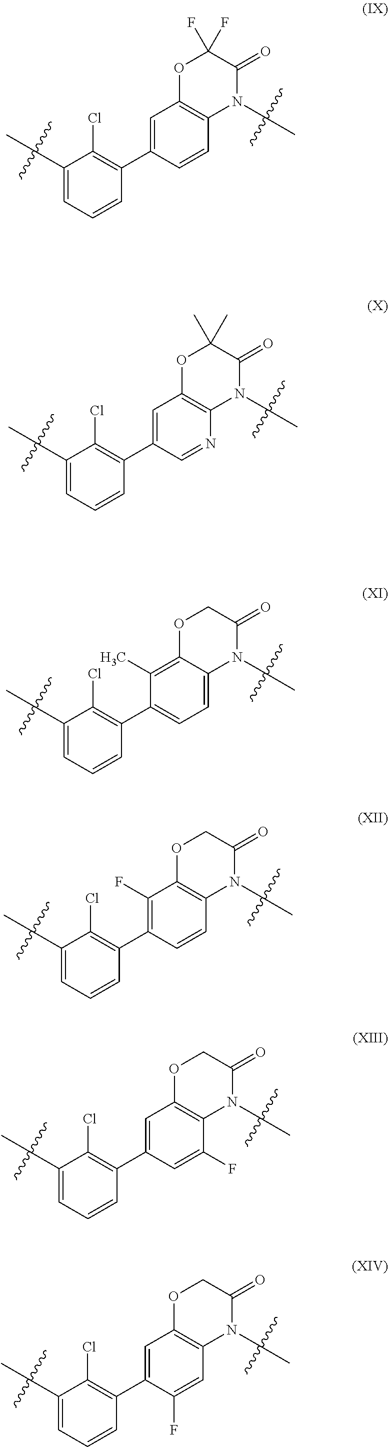 [SMILES: *c1cccc(-c2ccc3c(c2)OC(F)(F)C(=O)N3*)c1Cl ; *c1cccc(-c2cnc3c(c2)OC(C)(C)C(=O)N3*)c1Cl ; *c1cccc(-c2ccc3c(c2C)OCC(=O)N3*)c1Cl ; *c1cccc(-c2ccc3c(c2F)OCC(=O)N3*)c1Cl ; *c1cccc(-c2cc(F)c3c(c2)OCC(=O)N3*)c1Cl ; *c1cccc(-c2cc3c(cc2F)N(*)C(=O)CO3)c1Cl] 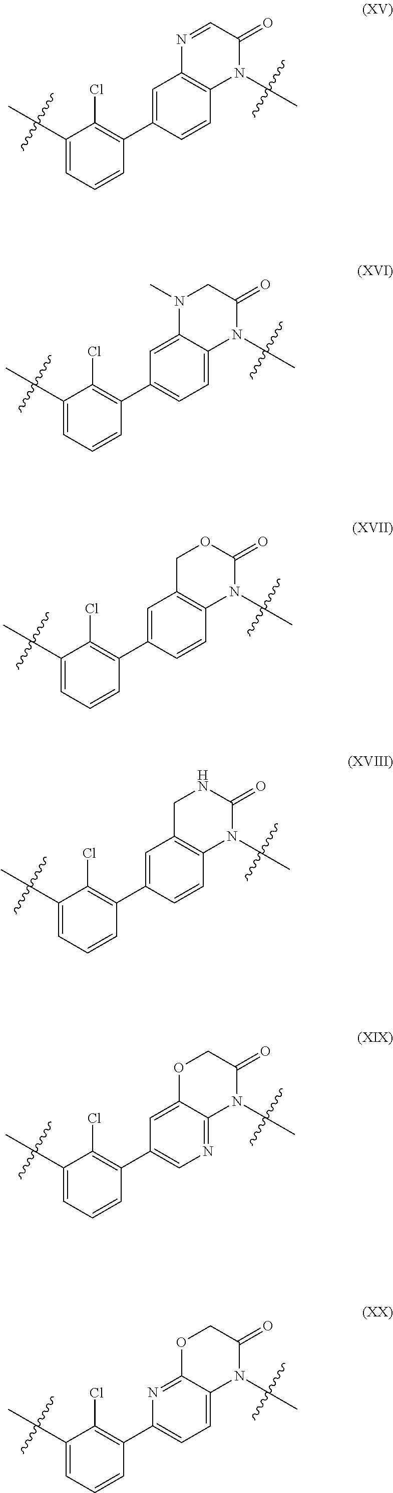 [SMILES: *c1cccc(-c2ccc3c(c2)ncc(=O)n3*)c1Cl ; *c1cccc(-c2ccc3c(c2)N(C)CC(=O)N3*)c1Cl ; *c1cccc(-c2ccc3c(c2)COC(=O)N3*)c1Cl ; *c1cccc(-c2ccc3c(c2)CNC(=O)N3*)c1Cl ; *c1cccc(-c2cnc3c(c2)OCC(=O)N3*)c1Cl ; *c1cccc(-c2ccc3c(n2)OCC(=O)N3*)c1Cl] 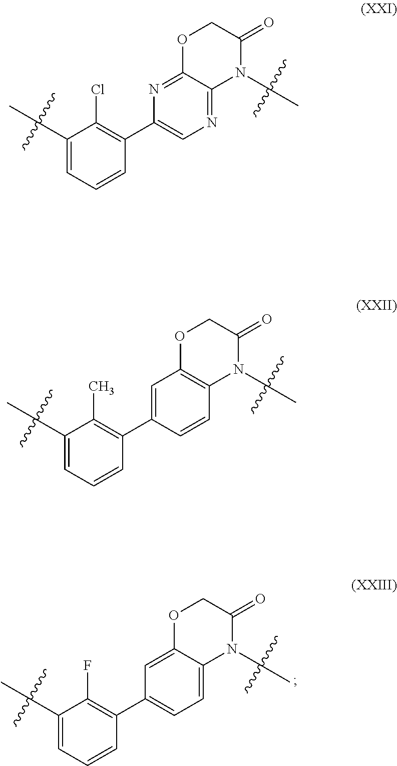 [SMILES: *c1cccc(-c2cnc3c(n2)OCC(=O)N3*)c1Cl ; *c1cccc(-c2ccc3c(c2)OCC(=O)N3*)c1C ; *c1cccc(-c2ccc3c(c2)OCC(=O)N3*)c1F]
preferably, wherein the compound comprises the core structures of: 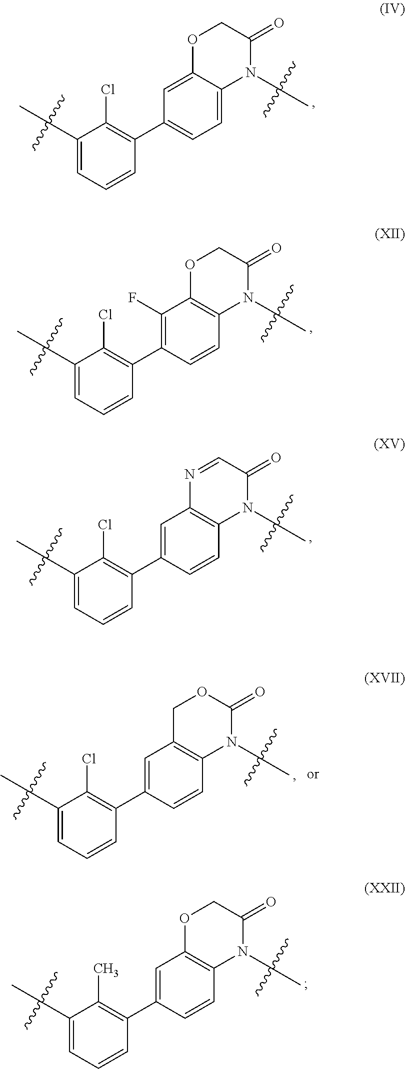
preferably, wherein L₁ and L₂ each is independently C₁-C₃ alkyl.

4. (canceled)

5. The compound of claim 1, wherein W₁ and W₂ each is independently a type I side chain, wherein type I side chains consist of

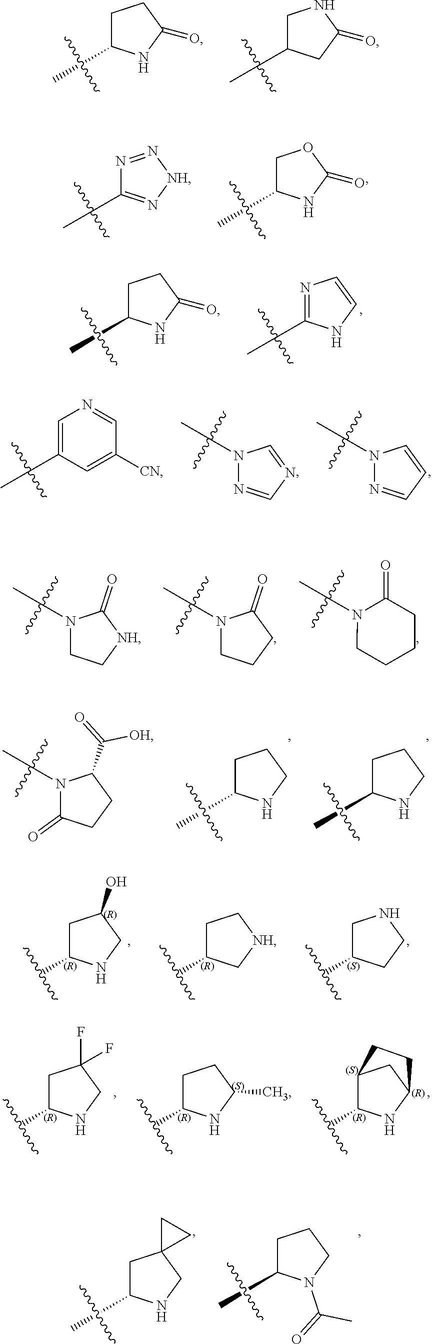

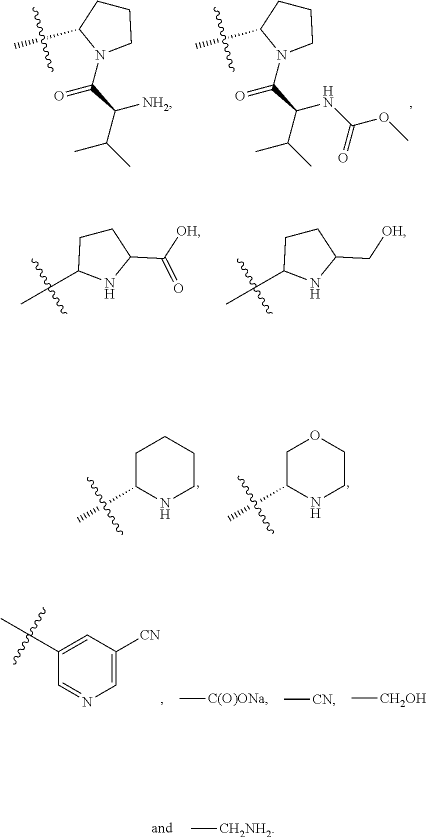

6. The compound of claim 1, wherein W₁ and W₂ each is independently a type II side chain, wherein type II side chains for W₁ have the general formula of: 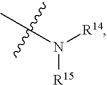
wherein R¹⁴ and R¹⁵ are independently —H, alkyl, or substituted alkyl,
wherein type II side chains for W₂ have the general formula of: 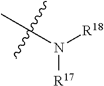
wherein R¹⁶ and R¹⁷ are independently —H, alkyl, or substituted alkyl;
preferably, wherein R¹⁴ is one of 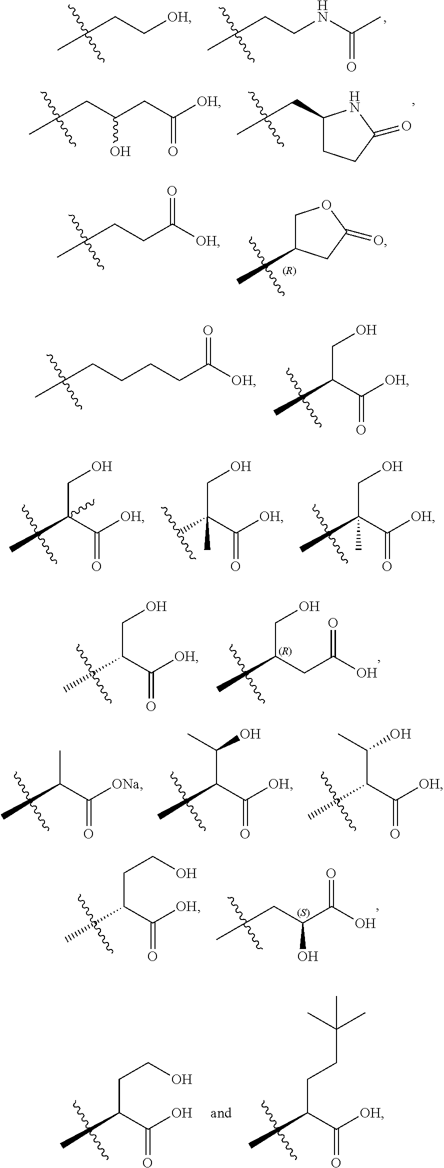
R¹⁵ is independently —H, alkyl, or substituted alkyl,
wherein R¹⁶ is one of 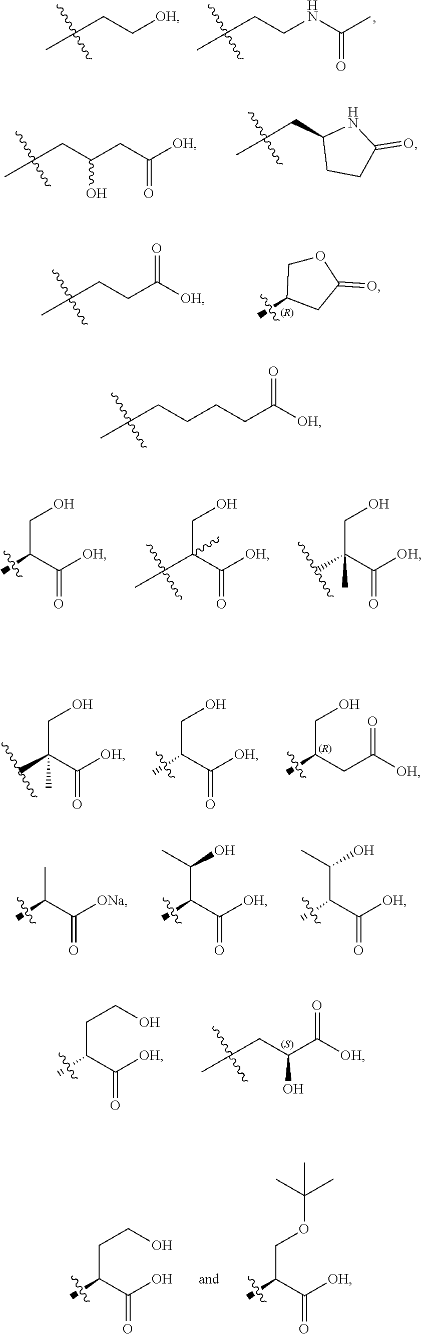
independently —H, alkyl, or substituted alkyl;
preferably, wherein R¹⁴ and R¹⁶ each is independently selected from the group of 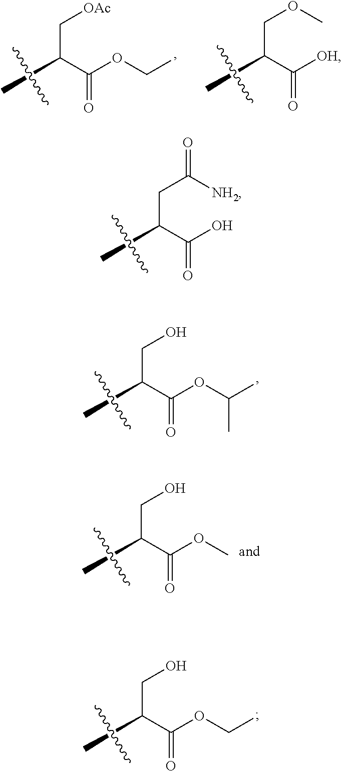
preferably, wherein W₁ or W₂ is L-serine;
preferably, wherein both W₁ and W₂ are L-serine;
preferably, wherein W₁ or W₂ is an ester of L-serine;
preferably, wherein both W₁ and W₂ are ester of L-serine;
preferably, wherein W₁ and/or W₂ is an ester of L-serine;
preferably, wherein W₁ and W₂ is independently selected from the group consisting of: 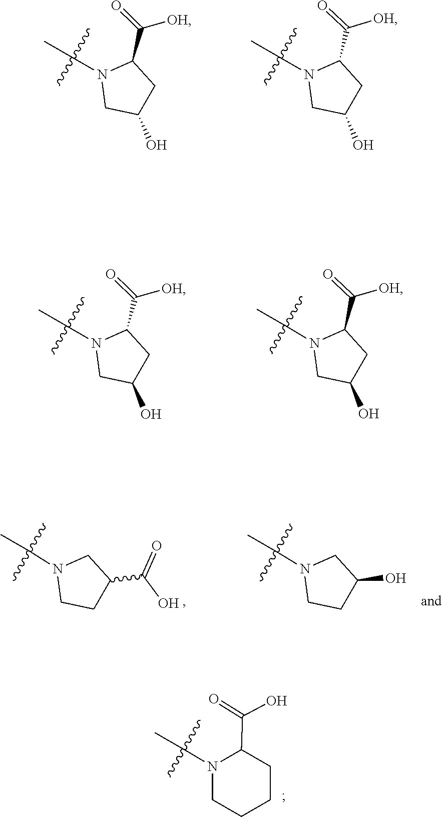
preferably, wherein W₁ is 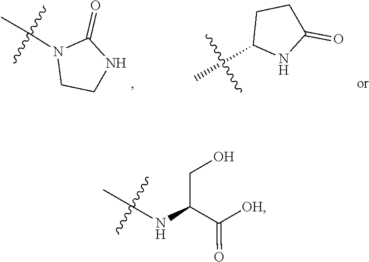
and
W₂ is H, and L₂ is absent (i.e., m=0);
W₁ is 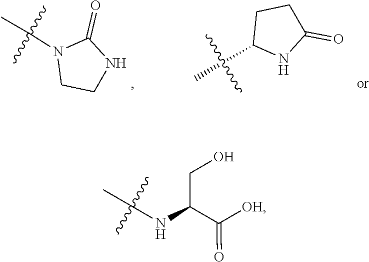
and W₂ is a type I or type II side chain;
preferably, wherein
W₁ is 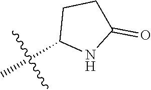
and W₂ is a type I or type II side chain;
preferably, wherein
W₁ is 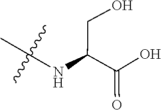 [SMILES: *N[C@@H](CO)C(=O)O]
and W₂ is a type I or type II side chain;
preferably, wherein
W₁ is 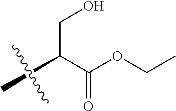
and W₂ is a type I or type II side chain;
preferably, wherein W₁ is 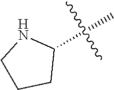
and W₂ is 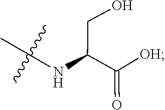 [SMILES: *N[C@@H](CO)C(=O)O]
preferably, wherein L₁ and L₂ each is independently a C₁-C₃ alkyl.

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. The compound of claim 1, wherein the compound is selected from the group consisting of Compound Nos. 281, 303, 448, 338, 354, 361, 363, 372, 376, 381, 382, 388, 395, 396, 401, 402, 457, 466, 481, 445, 449, 454, 460, 461, 465, 468, 469, 470, 471, 475, 476, 478, 482, 483, 484, 490, 491, 493, 513, 527, 528, 536, 537, 541, 545, 551, 552, 553, 560, 566, 570, 571, 573, 588, 479, 480, 563, 550, 554, 589, 561, 557, 586, 585, 514, 373, 374, 375, 378, 384, 386, 427, 394, 397, 408, 411, 503, 530, 531, 535, 539, 540, 412, 413, 415, 459, 422, 430, 472, 567, 529, 558, 559, 538, 429, 385, 533, 516, 515, 542, 488, 489, 492, 450, 517 of Table 1, or pharmaceutically acceptable salts thereof, stereoisomers thereof, mixtures of stereoisomers thereof, or tautomers thereof;
preferably, wherein the compound is selected from the group consisting of Compound Nos. 338, 361, 411, 465, 475, 483, 529, 533, 537, 541, 554, 585, 613, 614, 619, 620, 628, 633, 648, 649 of Table 1, or pharmaceutically acceptable salts thereof, stereoisomers thereof mixtures of stereoisomers thereof, or tautomer thereof.

23. (canceled)

25. The compound of claim 1, wherein the compound is

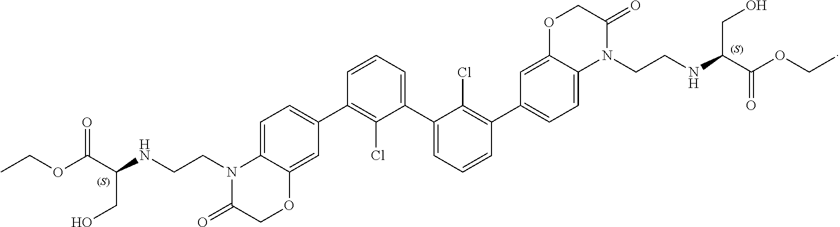

26. A method for treating a disease or condition relating to the interaction between PD-L1 and PD-1 in a subject, comprising the step of:
administering to the subject, an effective amount of the compound of claim 1, or a pharmaceutically acceptable salt, stereoisomer, mixture of stereoisomers, solvate, or tautomer thereof.

27. The method of claim 26, wherein the disease is cancer.

28. A pharmaceutical composition which contain the compound of claim 1, or a pharmaceutically acceptable salt, stereoisomer, mixture of stereoisomers, solvate, or tautomer thereof.

29. A method for treating a disease or condition relating to the interaction between PD-L1 and PD-1 in a subject, comprising the step of:
administering to the subject, an effective amount of the pharmaceutical composition of claim 28.

30. The method of claim 29, wherein the disease is cancer.
